SEQUENCE LISTING (4 sequences)

SEQ ID NO: 1 - protein, 112 aa, synthetic construct
DIVMTQSPDSLAVSLGERATINCKSSQSLLHSRSRKNYLAWYQQKPGQPPKLLIYWASTRESGVPDRFSGSGSGTDFTLTISSLQAEDVAVYYCKQSYNLLSFGQGTKLEIK

SEQ ID NO: 2 - protein, 116 aa, synthetic construct
QIQLVQSGSELKKPGASVKVSCKASGYTFTKYSMHWVRQAPGQGLEWMARINTETGNPTYADGFRGRFVVSLDTSVSTAYLQISSLKAEDTAVYYCAIFYYGRDTWGQGTTVTVSS

SEQ ID NO: 3 - protein, 219 aa, synthetic construct
DIVMTQSPDSLAVSLGERATINCKSSQSLLHSRSRKNYLAWYQQKPGQPPKLLIYWASTRESGVPDRFSGSGSGTDFTLTISSLQAEDVAVYYCKQSYNLLSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 443 aa, synthetic construct
QIQLVQSGSELKKPGASVKVSCKASGYTFTKYSMHWVRQAPGQGLEWMARINTETGNPTYADGFRGRFVVSLDTSVSTAYLQISSLKAEDTAVYYCAIFYYGRDTWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0195647 A1
(43) Pub. Date: Jun. 19, 2025

(54) ANTI-alpha-V-beta-8 INTEGRIN ANTIBODIES AND USES THEREOF

(71) Applicant: Corbus Pharmaceuticals, Inc., Norwood, MA (US)

(72) Inventors: Andrew KOLODZIEJ, Winchester, MA (US); Eric HAINES, Andover, MA (US); Richard J. MORSE, Bethel, CT (US); Tuhina BHATTACHARYA, Brockton, MA (US); Paul SLAMAN, Plainville, MA (US)

(21) Appl. No.: 18/980,943

(22) Filed: Dec. 13, 2024

Related U.S. Application Data
(60) Provisional application No. 63/609,748, filed on Dec. 13, 2023.

(51) Int. Cl. A61K 39/395; A61K 39/00; A61P 35/00; C07K 16/28

(52) U.S. Cl. CPC A61K 39/39558; A61P 35/00; C07K 16/2818; C07K 16/2839; A61K 2039/507; C07K 2317/24; C07K 2317/92

(57) ABSTRACT

The present disclosure provides antibodies or antigen-binding fragments thereof that bind specifically to integrin αvβ8. The disclosure also provides methods for enhancing an immune response in a subject. The disclosure also provides methods for treating or preventing αvβ8-associated disorders. In some embodiments, the antibodies are suitable for use (e.g., therapeutic use) in a human subject.

### SEQUENCE LISTING

The instant application contains a Sequence Listing which has been submitted electronically in XML file format and is hereby incorporated by reference in its entirety. Said XML copy, created on Dec. 6, 2024, is named “51186-108002_Sequence_Listing12_6_24” and is 5,232 bytes in size.

### BACKGROUND

Interactions between cells and their extracellular microenvironment are primarily mediated by a family of cell surface receptors known as integrins. Integrins are heterodimeric transmembrane receptors composed of α and β subunits that can combine to form 24 different integrin heterodimers. Integrins regulate cytoskeletal dynamics, thereby influencing a number of crucial cellular processes, for example, cell adhesion, migration and differentiation. Integrins also have a key role in the activation of growth factors such as transforming growth factor beta (TGF-β). Integrin αvβ8 binds to the latency associated domain of TGF-β (LAP) and mediates the activation of TGF-β. Accordingly, integrin αvβ8 is a target for therapeutic intervention and there is a need for therapeutic modalities that bind to integrin αvβ8 and which are suitable for use as therapeutics in human subjects.

### SUMMARY

The present disclosure provides antibodies that bind specifically to integrin αvβ8, and pharmaceutical compositions containing the antibodies. The disclosure also provides methods for enhancing an immune response in a subject. In some embodiments, the antibodies are suitable for use (e.g., therapeutic use) in a human subject.

In one aspect, the disclosure provides an antibody that binds to integrin αVβ8, the antibody comprising a light chain variable domain (VL) comprising an amino acid sequence of SEQ ID NO: 1 and a heavy chain variable domain (VH) comprising an amino acid sequence of SEQ ID NO: 2. In some embodiments, the antibody comprises a light chain amino acid sequence of SEQ ID NO: 3 and a heavy chain amino acid sequence of SEQ ID NO: 4.

In a second aspect, the disclosure provides a pharmaceutical composition comprising the antibody that binds to integrin αVβ8 and a pharmaceutically acceptable carrier or excipient.

In some embodiments, the pharmaceutical composition is an aqueous formulation having a pH of between 5.0 and 7.5 (e.g., a pH of 5.5±0.5, 6.0±0.5, 6.5±0.5, or 7.0±0.5) and the antibody is present at a concentration of between 20 mg/mL and 40 mg/mL (e.g., a concentration of 25±5 mg/mL, 30±5 mg/mL, or 35±5 mg/mL).

In some embodiments, the pharmaceutical composition comprises 10 to 30 mM (e.g., 15±5 mM, 20±5 mM, or 25±5 mM) phosphate buffer or histidine buffer.

In some embodiments, the pharmaceutical composition comprises 0.01% (w/w) to 0.1% (w/w) (e.g., 0.02±0.01%, 0.04±0.02%, 0.06±0.02%, or 0.08±0.02%) of a nonionic surfactant. In some embodiments, the nonionic surfactant is selected from polysorbate surfactants, polyglycolized glycerides, alkyl saccharides, and ester saccharides. In some embodiments, the nonionic surfactant is polyoxyethylene 20 sorbitan monolaurate.

In some embodiments, the pharmaceutical composition comprises from 40 to 60 mM (e.g., 45±5 mM, 50±5 mM or 55±5 mM) arginine or glutamate.

In some embodiments, the pharmaceutical composition comprises a nonionic tonicity agent. In some embodiments, the nonionic tonicity agent is selected from sucrose, mannitol, sorbitol, lactose, dextrose, trehalose, and glycerol. In some embodiments, the nonionic tonicity agent is sucrose, and the pharmaceutical composition comprises from 4% (w/w) to 7.5% (w/w) sucrose (e.g., 4.5±0.5%, 5.0±0.5%, 5.5±0.5%, 6.0±0.5%, 6.5±0.5%, or 7.0±0.5%).

In some embodiments, the pharmaceutical composition comprises less than 5 mM sodium chloride, or is free of sodium chloride.

In some embodiments, the antibody is present at a concentration of about 30 mg/mL.

In some embodiments, the pharmaceutical composition comprises 20 mM sodium phosphate, 5% (w/w) sucrose, 50 mM arginine, and 0.02% (w/w) polyoxyethylene 20 sorbitan monolaurate.

In some embodiments, the pharmaceutical composition is formulated at a pH of about 6.5.

In some embodiments, the pharmaceutical composition is formulated in a volume of about 10 mL.

In a third aspect, the disclosure provides a polynucleotide encoding the antibody that binds to integrin αVβ8 disclosed herein.

In a fourth aspect, the disclosure provides a vector comprising the polynucleotide encoding the antibody that binds to integrin αVβ8 disclosed herein. In some embodiments, the vector is an expression vector (e.g., a eukaryotic expression vector). In some embodiments, the vector is a viral vector (e.g., adenovirus (Ad), retrovirus, poxvirus, adeno-associated virus, baculovirus, or a herpes simplex virus).

In a fifth aspect, the disclosure provides a host cell comprising the vector disclosed herein.

In a sixth aspect, the disclosure provides a method of enhancing an immune response in a subject, the method comprising administering to the subject the antibody that binds to integrin αVβ8 or a pharmaceutical composition thereof described herein.

In some embodiments, the subject has cancer and the antibody that binds to integrin αVβ8 enhances the immune response against the cancer thereby treating the cancer. In some embodiments, the cancer is a solid tumor, a metastatic cancer, or a primary cancer. In some embodiments, the cancer is lung cancer, optionally non-small cell lung cancer or small-cell lung cancer; head and neck cancer, optionally head and neck squamous cell carcinoma; renal cell carcinoma, optionally renal clear cell carcinoma or kidney renal papillary cell carcinoma; breast cancer, optionally triple-negative breast cancer; ovarian cancer; pancreatic cancer; brain cancer, optionally gliomas; colorectal cancer; urothelial cancer; bile duct cancer; endometrial cancer; melanoma; cervical cancer; gastric cancer; hepatocellular carcinoma; glioblastoma; or esophageal cancer. In some embodiments, the cancer is an immune refractory cancer (e.g., an immune refractory cancer of any cancer listed herein).

In some embodiments, the subject has had disease progression after at least one line of therapy or has no other standard therapy of proven clinical benefit currently available. In some embodiments, the at least one line of therapy includes an immune checkpoint inhibitor. In some embodiments, the subject is resistant to treatment with an immune checkpoint inhibitor. In some embodiments, the subject has acquired resistance to treatment with an immune checkpoint inhibitor. In some embodiments, administering the antibody that binds to integrin αVβ8 or pharmaceutical composition thereof sensitizes the cancer to treatment with an immune checkpoint inhibitor.

In some embodiments, administering the antibody that binds to integrin αVβ8 or pharmaceutical composition thereof transforms an immunologically cold tumor into an immunologically hot tumor. In some embodiments, the method reduces tumor size or inhibits tumor growth. In some embodiments, the method alters the tumor microenvironment.

In some embodiments, the subject is further administered an immune checkpoint inhibitor. In some embodiments, the immune checkpoint inhibitor is a PD-(L)1 inhibitor. In some embodiments, the PD-(L)1 inhibitor is an anti-PD-1 antibody (e.g., cemiplimab, nivolumab, pembrolizumab, dostarlimab, toripalimab, tislelizumab, or retifanlimab). In some embodiments, the PD-(L)1 inhibitor is an anti-PD-L1 antibody (e.g., atezolizumab, avelumab, or durvalumab).

In some embodiments, the subject is further administered a CTLA-4 inhibitor (e.g., tremilimumab or ipilimumab).

In some embodiments, the subject is further administered a LAG-3 inhibitor (e.g., relatlimab).

In some embodiments, the subject is further administered an angiogenesis inhibitor (e.g., axitinib, tivozanib, vandetanib, nintedanib, sunitinib, or sorafenib). In some embodiments, the angiogenesis inhibitor is an anti-VEGF antibody (e.g., bevacizumab), an anti-VEGF trap, or an anti-VEGF receptor antibody (e.g., ramucirumab) In some embodiments, the subject is further administered a chemotherapeutic agent. In some embodiments, the chemotherapeutic agent is platinum chemotherapy (e.g., oxaliplatin, carboplatin, or cisplatin), trastuzumab, fluoropyrimidine, gemcitabine, irinotecan, 5-fluorouracil, or a taxane (e.g., docetaxel or paclitaxel).

In some embodiments, the subject is further administered immune-priming stereotactic body radiation therapy. In some embodiments, the immune-priming stereotactic body radiation therapy is administered at a dose of 8 Gy on Day 1, Day 3, and Day 5 of the one or more dosing cycles.

In some embodiments, the antibody that binds to integrin αVβ8 or a pharmaceutical composition thereof is administered at a dose of about 1 mg/kg to about 30 mg/kg (e.g., 5±4 mg/kg, 10±5 mg/kg, 15±5 mg/kg, 20±5 mg/kg, or 25±5 mg/kg). In some embodiments, the antibody that binds to integrin αVβ8 or a pharmaceutical composition thereof is administered at a dose of about 1 mg/kg to about 5 mg/kg. In some embodiments, the antibody that binds to integrin αVβ8 or a pharmaceutical composition thereof is administered at a dose of about 5 mg/kg to about 10 mg/kg. In some embodiments, the antibody that binds to integrin αVβ8 or a pharmaceutical composition thereof is administered at a dose of about 10 mg/kg to about 15 mg/kg. In some embodiments, the antibody that binds to integrin αVβ8 or a pharmaceutical composition thereof is administered at a dose of about 15 mg/kg to about 20 mg/kg. In some embodiments, the antibody that binds to integrin αVβ8 or a pharmaceutical composition thereof is administered at a dose of about 20 mg/kg to about 25 mg/kg. In some embodiments, the antibody that binds to integrin αVβ8 or a pharmaceutical composition thereof is administered at a dose of about 25 mg/kg to about 30 mg/kg.

In some embodiments, the length of each of the one or more dosing cycles is 21 days. In some embodiments, the antibody that binds to integrin αVβ8 or a pharmaceutical composition thereof is administered on Day 1 of each of the one or more dosing cycles. In some embodiments, the antibody that binds to integrin αVβ8 or a pharmaceutical composition thereof is administered on Day 1 and Day 8 of each of the one or more dosing cycles. In some embodiments, the antibody that binds to integrin αVβ8 or a pharmaceutical composition thereof is administered on Day 1, Day 8, and Day 15 of each of the one or more dosing cycles.

In some embodiments, the length of each of the one or more dosing cycles is 28 days. In some embodiments, the antibody that binds to integrin αVβ8 or a pharmaceutical composition thereof is administered on Day 1 and Day 15 of each of the one or more dosing cycles. In some embodiments, the antibody that binds to integrin αVβ8 or a pharmaceutical composition thereof is administered on Day 1, Day 8, Day 15, and Day 22 of each of the one or more dosing cycles. In some embodiments, the antibody that binds to integrin αVβ8 or a pharmaceutical composition thereof is administered on Day 1, Day 8, and Day 15 of each of the one or more dosing cycles.

In some embodiments, the antibody that binds to integrin αVβ8 or a pharmaceutical composition thereof is administered intravenously. In some embodiments, the antibody that binds to integrin αVβ8 or a pharmaceutical composition thereof is administered by intravenous infusion over 30±5 minutes.

### Definitions

To facilitate the understanding of this disclosure, a number of terms are defined below. Terms defined herein have meanings as commonly understood by a person of ordinary skill in the areas relevant to the disclosure. Terms such as “a”, “an,” and “the” are not intended to refer to only a singular entity, but include the general class of which a specific example may be used for illustration. The terminology herein is used to describe specific embodiments of the disclosure, but their usage does not limit the disclosure, except as outlined in the claims.

As used herein, the term “about” refers to a value that is no more than 10% above or below the value being described. For example, the term “about 5 nM” indicates a range of from 4.5 nM to 5.5 nM.

As used herein, any values provided in a range of values include both the upper and lower bounds and any values contained within the upper and lower bounds.

The terms “anti-αvβ8 antibody,” “αvβ8 specific antibody,” “αvβ8 antibody,” and “anti-αvβ8” are used synonymously herein to refer to an antibody that specifically binds to αvβ8. Similarly, an anti-β8 antibody, and like terms, refer to an antibody that specifically binds to β8. The anti-αvβ8 antibodies and anti-β8 antibodies described herein bind to the protein expressed on αvβ8 expressing cells.

An αvβ8-associated disorder is a condition characterized by the presence of αvβ8-expressing cells, either cells expressing an increased level of αvβ8, or increased number of αvβ8-expressing cells relative to a normal, non-diseased control. TGF-β-associated disorders (e.g., disorders characterized by higher than normal TGF-β activity) include αvβ8-associated disorders, as αvβ8 is involved in activating TGF-β in certain circumstances, as described herein.

“Nucleic acid” refers to deoxyribonucleotides or ribonucleotides and polymers thereof in either single- or double-stranded form and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA.

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “recombinant” when used with reference, e.g., to a cell, or nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid includes two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein includes two or more subsequences that are not found in the same relationship to each other in nature (e.g., a fusion protein).

The term “isolated,” when applied to a nucleic acid or protein, denotes that the nucleic acid or protein is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high performance liquid chromatography. A protein that is the predominant species present in a preparation is considered substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “antibody” refers to a polypeptide including a framework region encoded by an immunoglobulin gene, or fragments thereof, that specifically bind and recognize an antigen, e.g., human αvβ8, a particular cell surface marker, or any desired target. Typically, the “variable region” contains the antigen-binding region of the antibody (or its functional equivalent) and is most critical in specificity and affinity of binding. See Paul, Fundamental Immunology (2003).

The various antibodies described herein can be produced by enzymatic or chemical modification of the intact antibodies, or synthesized de novo using recombinant DNA methodologies, or identified using phage display libraries (see, e.g., McCafferty et al., Nature 348:552-554, 1990).

A “monoclonal antibody” refers to a clonal preparation of antibodies with a single binding specificity and affinity for a given epitope on an antigen. A “polyclonal antibody” refers to a preparation of antibodies that are raised against a single antigen, but with different binding specificities and affinities.

As used herein, “variable region” or “V-region” refers to an antibody variable region domain including the segments of Framework 1, CDR1, Framework 2, CDR2, Framework 3, CDR3, and Framework 4. These segments are included in the V-segment as a consequence of rearrangement of the heavy chain and light chain V-region genes during B-cell differentiation.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson and Wu, Nucleic Acids Res. 2000 Jan. 1; 28(1): 214-218 and Johnson et al., Nucleic Acids Res., 29:205-206 (2001); Chothia & Lesk, (1987) J. Mol. Biol. 196, 901-917; Chothia et al. (1989) Nature 342, 877-883; Chothia et al. (1992) J. Mol. Biol. 227, 799-817; Al-Lazikani et al., J. Mol. Biol. 1997, 273(4)). Unless otherwise indicated, CDRs are determined according to Kabat. Definitions of antigen combining sites are also described in the following: Ruiz et al. Nucleic Acids Res., 28, 219-221 (2000); and Lefranc Nucleic Acids Res. January 1; 29(1):207-9 (2001); MacCallum et al., J. Mol. Biol., 262: 732-745 (1996); and Martin et al, Proc. Natl Acad. Sci. USA, 86, 9268-9272 (1989); Martin, et al, Methods Enzymol., 203:121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

A “chimeric antibody” is an antibody molecule in which (a) the constant region, or a portion thereof, is altered, replaced, or exchanged so that the antigen binding site (variable region, CDR, or portion thereof) is linked to a constant region of a different or altered class, effector function and/or species, or an entirely different molecule which confers new properties to the chimeric antibody (e.g., an enzyme, toxin, hormone, growth factor, drug, etc.); or (b) the variable region, or a portion thereof, is altered, replaced or exchanged with a variable region having a different or altered antigen specificity (e.g., CDR and framework regions from different species).

The antibody binds to an “epitope” on the antigen. The epitope is the specific antibody binding interaction site on the antigen, and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures.

The term “specifically bind” refers to a molecule (e.g., antibody or antibody fragment) that binds to a target with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity. For example, an antibody that specifically binds β8 will typically bind to β8 with at least a 2-fold greater affinity than a non-β8 target.

The term “binds” with respect to a cell type (e.g., an antibody that binds fibrotic cells, hepatocytes, chondrocytes, etc.), typically indicates that an agent binds a majority of the cells in a pure population of those cells. For example, an antibody that binds a given cell type typically binds to at least ⅔ of the cells in a population of the indicated cells (e.g., 75, 80, 85, 90, 91, 92, 93, 94, 95, 96, 97, 98, 99, or 100%). One of skill will recognize that some variability will arise depending on the method and/or threshold of determining binding.

As used herein, a first antibody, or an antigen-binding portion thereof, “competes” for binding to a target with a second antibody, or an antigen-binding portion thereof, when binding of the second antibody with the target is detectably decreased in the presence of the first antibody compared to the binding of the second antibody in the absence of the first antibody. The alternative, where the binding of the first antibody to the target is also detectably decreased in the presence of the second antibody, can, but need not be the case. That is, a second antibody can inhibit the binding of a first antibody to the target without that first antibody inhibiting the binding of the second antibody to the target. However, where each antibody detectably inhibits the binding of the other antibody to its cognate epitope or ligand, whether to the same, greater, or lesser extent, the antibodies are said to “cross-compete” with each other for binding of their respective epitope(s). Both competing and cross-competing antibodies are encompassed by the present disclosure. The term “competitor” antibody can be applied to the first or second antibody as can be determined by one of skill in the art. In some cases, the presence of the competitor antibody (e.g., the first antibody) reduces binding of the second antibody to the target by at least 10%, e.g., 20%, 30%, 40%, 50%, 60%, 70%, 80%, or more, e.g., so that binding of the second antibody to target is undetectable in the presence of the first (competitor) antibody.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

A “labeled” molecule (e.g., nucleic acid, protein, or antibody) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule.

The term “diagnosis” refers to a relative probability that a disorder such as cancer or an inflammatory condition is present in the subject. Similarly, the term “prognosis” refers to a relative probability that a certain future outcome may occur in the subject. For example, prognosis can refer to the likelihood that an individual will develop a TGF-β or αvβ8 associated disorder, have recurrence, or the likely severity of the disease (e.g., severity of symptoms, rate of functional decline, survival, etc.). The terms are not intended to be absolute, as will be appreciated by any one of skill in the field of medical diagnostics.

“Biopsy” or “biological sample from a patient” as used herein refers to a sample obtained from a patient having, or suspected of having, a TGF-β or αvβ8 associated disorder. In some embodiments, the sample may be a tissue biopsy, such as needle biopsy, fine needle biopsy, surgical biopsy, etc. The sample can also be a blood sample or blood fraction, e.g., white blood cell fraction, serum, or plasma. The sample can include a tissue sample harboring a lesion or suspected lesion, although the biological sample may also be derived from another site, e.g., a site of suspected metastasis, a lymph node, or from the blood. In some cases, the biological sample may also be from a region adjacent to the lesion or suspected lesion.

A “biological sample” can be obtained from a patient, e.g., a biopsy, from an animal, such as an animal model, or from cultured cells, e.g., a cell line or cells removed from a patient and grown in culture for observation. Biological samples include tissues and bodily fluids, e.g., blood, blood fractions, lymph, saliva, urine, feces, etc.

The terms “therapy,” “treatment,” and “amelioration” refer to any reduction in the severity of symptoms. In the case of treating an inflammatory condition, the treatment can refer to reducing, e.g., blood levels of inflammatory cytokines, blood levels of active mature TGF-β, pain, swelling, recruitment of immune cells, etc. In the case of treating cancer, treatment can refer to reducing, e.g., tumor size, number of cancer cells, growth rate, metastatic activity, cell death of non-cancer cells, etc. As used herein, the terms “treat” and “prevent” are not intended to be absolute terms. Treatment and prevention can refer to any delay in onset, amelioration of symptoms, improvement in patient survival, increase in survival time or rate, etc. Treatment and prevention can be complete (no detectable symptoms remaining) or partial, such that symptoms are less frequent of severe than in a patient without the treatment described herein. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” “therapeutically effective amount,” etc. refer to that amount of the therapeutic agent sufficient to ameliorate a disorder, as described above. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable as having utility in pharmaceutical compositions. A “pharmaceutical composition” will generally include agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For the present disclosure, the dose can refer to the concentration of the antibody or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

“Cancer”, “tumor,” “transformed” and like terms include precancerous, neoplastic, transformed, and cancerous cells, and can refer to a solid tumor, or a non-solid cancer (see, e.g., Edge et aL. AJCC Cancer Staging Manual (7^{th} ed. 2009); Cibas and Ducatman Cytology: Diagnostic principles and clinical correlates (3^{rd} ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3^{rd} ed. 1994)). Although transformation can arise from infection with a transforming virus and incorporation of new genomic DNA, or uptake of exogenous DNA, it can also arise spontaneously or following exposure to a carcinogen.

The term “cancer” can refer to carcinomas, sarcomas, adenocarcinomas, lymphomas, leukemias, solid and lymphoid cancers, etc. Examples of different types of cancer include, but are not limited to, lung cancer (e.g., non-small cell lung cancer or small-cell lung cancer), ovarian cancer, prostate cancer, colorectal cancer, liver cancer (i.e., hepatocarcinoma), renal cancer (i.e., renal cell carcinoma, optionally renal clear cell carcinoma or kidney renal papillary cell carcinoma), bladder cancer, breast cancer (e.g., triple-negative breast cancer), urothelial cancer, endometrial cancer, thyroid cancer, pleural cancer, pancreatic cancer, brain cancer, optionally gliomas; uterine cancer, cervical cancer, testicular cancer, anal cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma; head and neck cancer (e.g., head and neck squamous cell carcinoma), blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia (AML), chronic myeloid leukemia (CML), hepatocellular carcinoma, glioblastoma, and multiple myeloma. In some embodiments, the antibody compositions and methods described herein can be used for treating cancer.

As used interchangeably herein, the terms “immune refractory,” “immune evasive,” or “cold tumor” refers to a tumor, cancer, or patient having a tumor or cancer, for which a prior immunotherapy, such as an immune checkpoint inhibitor, has been found ineffective or intolerable. For example, a patient having an immune refractory cancer includes a patient who has previously been administered an immunotherapy, such as an immune checkpoint inhibitor, and the immunotherapy has been found ineffective or found not adequately effective to slow or halt the progression of the disease or to alleviate symptoms associated with the progression of the disease. Immune refractory cancers include cancers that have become resistant to or desensitized to treatment with immunotherapy (e.g., the effectiveness of an immunotherapy, such as an immune checkpoint inhibitor, previously administered to the patient is diminished over time). Immune refractory cancers can be identified by methods known to those of skill in the art or by methods described herein. For example, immune refractory cancers may be characterized by low immune cell infiltrate in one or more tumors. Low immune cell infiltrate may include a decrease or absence of lymphocytes; a decrease or absence of tumor-infiltrating lymphocytes (TILs); a decrease or absence of dendritic cells; a decrease or absence of myeloid cells; a decrease or absence of natural killer (NK) cells; a decrease or absence of macrophages; a decrease or absence of T cells; a decrease or absence of CD8+ T cells; a decrease or absence of CD4+ T cells; or a decrease or absence of CD4+/CD8+ T cells. See, e.g., Chen and Mellman, Nature, 541:321 (2017). By contrast, a “hot tumor” refers to a tumor, cancer, or patient having a tumor or cancer that is not immune refractory. Cells having a low cytotoxic T cell count can be characterized as an “immune desert” or “immune excluded.” In some embodiments, cells having a cytotoxic T cell count of less than 1% live cells are considered an immune desert or immune excluded. In some embodiments, cells having a cytotoxic T cell count of less than 0.5% live cells are considered an immune desert or immune excluded. In some embodiments, cells having a cytotoxic T cell count of less than 0.25% live cells are considered an immune desert or immune excluded. It is also recognized that other cell types such as cancer-associated fibroblasts (CAFs) can contribute to immune exclusion by secreting cytokines such as TGFb into the tumor microenvironment and by synthesizing the fibrotic extracellular matrix that underlies desmoplasia, characteristic of many resistant tumors (Grauel et al., Nature Communications 11, 6315 (2020); Dominguez et al., Cancer Discovery 10, 232 (2020). For immune cells as well as CAFs, it is also recognized that the activation state of specific immune cells (defined by markers such as IFNg, Granzyme B, CD25, CD69) and their genetic program further defines the responsiveness of the tumor to immunotherapy (Cogdill et al., British Journal Cancer 117,1 (2017); Mariathasan, S. et al., Nature 554, 544 (2018)) and inherent restraints to anti-tumor immunity.

As used herein, the term “resistant to treatment” refers to a treatment of a disorder with a therapeutic agent, where the therapeutic agent is ineffective or where the therapeutic agent was previously effective and has become less effective overtime. Resistance to treatment includes acquired resistance to treatment, which refers to a decrease in the efficacy of a treatment over a period of time where the subject is being administered the therapeutic agent. Acquired resistance to treatment may result from the acquisition of a mutation in a target protein, or in multiple proteins and genetic loci, that renders the treatment ineffective or less effective. Accordingly, resistance to treatment may persist even after cessation of administration of the therapeutic agent. In particular, a cancer may become resistant to treatment with an immune checkpoint inhibitor following treatment with an immune checkpoint inhibitor. Such cancers are also referred to herein as “immune refractory.” Measurement of a decrease in the efficacy of the treatment will depend on the disorder being treated, and such methods are known to those of skill in the art. For example, efficacy of a cancer treatment may be measured by the progression of the disease. An effective treatment may slow or halt the progression of the disease. A cancer that is resistant to treatment with a therapeutic agent, e.g., an immune checkpoint inhibitor, may fail to slow or halt the progression of the disease.

As used herein, “disease progression” refers to a worsening of a disease. In some instances, disease progression is radiographic disease progression, e.g., as defined by growth of existing lesions, new lesions, or recurrence of previously resolved lesions. Disease progression (e.g., radiographic disease progression) can be determined by RECIST v1.1. In some embodiments, disease progression (or lack of disease progression) is confirmed by a confirmatory scan and/or pathology.

The term “co-administer” or “combination therapy” refers to the simultaneous presence of two active agents in the blood of an individual. Active agents that are co-administered can be concurrently or sequentially delivered.

As used herein, the term “PD-(L)1 inhibitor” refers to a molecule that inhibits the interaction of a PD-1 axis binding partner with either one or more of its binding partner, so as to remove T-cell dysfunction resulting from signaling on the PD-1 signaling axis, with a result being to restore or enhance T-cell function (e.g., proliferation, cytokine production, target cell killing). As used herein, a PD-(L)1 inhibitor may include an anti-PD-1 antibody (e.g., cemiplimab, nivolumab, pembrolizumab, dostarlimab, toripalimab, tislelizumab, or retifanlimab) or an anti-PD-L1 antibody (e.g., atezolizumab, avelumab, or durvalumab).

A “CTLA-4 inhibitor” refers to a compound, such as an antibody, capable of inhibiting the activity of the protein that in humans is encoded by the CTLA4 gene. CTLA-4 inhibitors include tremilimumab and ipilimumab.

A “LAG-3 inhibitor” refers to a compound, such as an antibody, capable of inhibiting the activity of the protein that in humans is encoded by the LAG-3 gene. LAG-3 inhibitors include relatlimab.

An “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. The angiogenesis inhibitor may include those agents that bind and block the angiogenic activity of the angiogenic factor or its receptor. For example, an angiogenesis inhibitor is an antibody or other antagonist to an angiogenic agent, e.g., antibodies to VEGF-A or the VEGF-A receptor (e.g., KDR receptor or Flt-1 receptor), anti-PDGFR inhibitors such as GLEEVEC™ (imatinib mesylate). Angiogenesis inhibitors also include native angiogenesis inhibitors, e.g., angiostatin, endostatin, etc. See, for example, Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) and Sato Int. J. Clin. Oncol., 8:200-206 (2003). Angiogenesis inhibitors also include axitinib, tivozanib, vandetanib, nintedanib, sunitinib, and sorafenib.

An “anti-VEGF antibody” is an antibody that binds to VEGF. In some embodiments the anti-VEGF antibody is bevacizumab, also known as “rhuMAb VEGF,” “BV,” or “AVASTIN®.” Bevacizumab is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 Daltons and is glycosylated. Bevacizumab and other humanized anti-VEGF antibodies are further described in U.S. Pat. No. 6,884,879, issued Feb. 26, 2005, the entire disclosure of which is expressly incorporated herein by reference. Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., (Journal of Immunological Methods 288:149-164, 2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183, and Q89.

An “anti-VEGF receptor antibody” refers to an antibody that binds to a VEGF receptor. Anti-VEGF receptor antibodies may include antibodies that bind VEGFR-2 (e.g., ramucirumab) and antibodies that bind VEGFR-1 (e.g., IMC-18F1).

An “anti-VEGF trap” refers to a polypeptide that binds VEGF. Anti-VEGF traps may include sequences from the native receptors VEGFR1 and VEGFR2. In some embodiments, the anti-VEGF trap may be aflibercept.

As used herein, the term “chemotherapeutic agent” refers to chemical compounds useful in the treatment of cancer. Examples of chemotherapeutic agents include alkylating agents, intercalating agents, antimicrotubule agents, antimitotics, antimetabolites, antiproliferative agents, antibiotics, immunomodulatory agents, anti-inflammatories, kinases inhibitors, antivascular agents, oestrogenic and androgenic hormones. Chemotherapeutic agents include platinum chemotherapy (e.g., oxaliplatin, carboplatin, and cisplatin), trastuzumab, fluoropyrimidine, gemcitabine, irinotecan, 5-fluorouracil, and taxanes (e.g., docetaxel and paclitaxel).

As used herein, the term “immune-priming stereotactic body radiation therapy” (SBRT) refers to highly potent ablative doses of radiation which stimulate expression of MHC class 1 molecules for enhanced tumor recognition, release of tumor damage-associated molecular patterns (DAMP's) which may lead to maturation of dendritic cells, stimulation of cytotoxic T-cell activity via dendritic-cell antigen presentation, and enhanced anti-tumor immunomodulation.

The formulations of the invention can include a nonionic surfactant. Examples of nonionic surfactants include polysorbate surfactants, polyglycolized glycerides, alkyl saccharides, and ester saccharides.

As used herein, “polysorbate surfactant” refers to oily liquids derived from pegylated sorbitan esterified with fatty acids. Common brand names for Polysorbates include Alkest, Canarcel and Tween. Polysorbate surfactants include, without limitation, polyoxyethylene 20 sorbitan monolaurate (TWEEN 20), polyoxyethylene (4) sorbitan monolaurate (TWEEN 21), polyoxyethylene 20 sorbitan monopalmitate (TWEEN 40), polyoxyethylene 20 sorbitan monostearate (TWEEN 60); and polyoxyethylene 20 sorbitan monooleate (TWEEN 80).

By “polyglycolized glyceride” is meant a polyethylene glycol glyceride monoester, a polyethylene glycol glyceride diester, a polyethylene glycol glyceride triester, or a mixture thereof containing a variable amount of free polyethylene glycol, such as a polyethylene glycol-oil transesterification product. The polyglycolized glyceride can include either monodisperse (i.e., single molecular weight) or polydisperse polyethylene glycol moieties of a predetermined size or size range (e.g., PEG2 to PEG 40). Polyethylene glycol glycerides include, for example: PEG glyceryl caprate, PEG glyceryl caprylate, PEG-20 glyceryl laurate (Tagat® L, Goldschmidt), PEG-30 glyceryl laurate (Tagat® L2, Goldschmidt), PEG-15 glyceryl laurate (Glycerox L series, Croda), PEG-40 glyceryl laurate (Glycerox L series, Croda), PEG-20 glyceryl stearate (Capmul® EMG, ABITEC), and Aldo® MS-20 KFG, Lonza), PEG-20 glyceryl oleate (Tagat® 0, Goldschmidt), and PEG-30 glyceryl oleate (Tagat® 02, Goldschmidt). Caprylocapryl PEG glycerides include, for example, caprylic/capric PEG-8 glyceride (Labrasol®, Gattefosse), caprylic/capric PEG-4 glyceride (Labrafac® Hydro, Gattefosse), and caprylic/capric PEG-6 glyceride (SOFTIGEN®767, Huls). Oleoyl PEG glyceride include, for example oleoyl PEG-6 glyceride, (Labrafil M1944 CS, Gattefosee). Lauroyl PEG glycerides includes, for example, lauroyl PEG-32 glyceride (Gelucire® ELUCIRE 44/14, Gattefosse). Stearoyl PEG glycerides include, for example stearoyl PEG-32 glyceride (Gelucrire 50/13, Gelucire 53/10, Gattefosse). PEG castor oils include PEG-3 castor oil (Nikkol CO-3, Nikkol, PEG-5, 9, and 16 castor oil (ACCONON CA series, ABITEC), PEG-20 castor oil, (Emalex C-20, Nihon Emulsion), PEG-23 castor oil (Emulgante EL23), PEG-30 castor oil (Incrocas 30, Croda), PEG-35 castor oil (Incrocas-35, Croda), PEG-38 castor oil (Emulgante EL 65, Condea), PEG-40 castor oil (Emalex C-40, Nihon Emulsion), PEG-50 castor oil (Emalex C-50, Nihon Emulsion), PEG-56 castor oil (Eumulgin® PRT 56, Pulcra SA), PEG-60 castor oil (Nikkol CO-60TX, Nikkol, PEG-100 castor oil, PEG-200 castor oil (Eumulgin® PRT 200, Pulcra SA), PEG-5 hydrogenated castor oil (Nikkol HCO-5, Nikkol, PEG-7 hydrogenated castor oil (Cremophor W07, BASF), PEG-10 hydrogenated castor oil (Nikkol HCO-10, Nikkol, PEG-20 hydrogenated castor oil (Nikkol HCO-20, Nikkol, PEG-25 hydrogenated castor oil (Simulsol® 1292, Seppic), PEG-30 hydrogenated castor oil (Nikkol HCO-30, Nikkol, PEG-40 hydrogenated castor oil (Cremophor RH 40, BASF), PEG-45 hydrogenated castor oil (Cerex ELS 450, Auschem Spa), PEG-50 hydrogenated castor oil (Emalex HC-50, Nihon Emulsion), PEG-60 hydrogenated castor oil (Nikkol HCO-60, Nikkol, PEG-80 hydrogenated castor oil (Nikkol HCO-80, Nikkol, and PEG-100 hydrogenated castor oil (Nikkol HCO-100, Nikkol. Additional polyethylene glycol-oil transesterification products include, for example, stearoyl PEG glyceride (Gelucire® 50/13, Gattefosse). The polyglycolized glycerides useful in the formulations of the invention can include polyethylene glycol glyceride monoesters, diesters, and/or triesters of acetic, propionic, butyric, valeric, hexanoic, heptanoic, caprylic, nonanoic, capric, lauric, myristic, palmitic, heptadecanoic, stearic, arachidic, behenic, lignoceric, α-linolenic, stearidonic, eicosapentaenoic, docosahexaenoic, linoleic, γ-linolenic, dihomo-γ-linolenic, arachidonic, oleic, elaidic, eicosenoic, erucic, or nervonic acid, or mixtures thereof. The polyglycol moiety in a polyglycolized glyceride can be polydisperse; that is, they can have a variety of molecular weights.

As used herein, “alkyl saccharide” refers to sugar ethers of a hydrophobic alkyl group (e.g., typically from 9 to 24 carbon atoms in length). Alkyl saccharides include alkyl glycosides and alkyl glucosides. In particular embodiments, the cefepime is formulated with a C8-14 alkyl ether of a sugar. Alkyl glycosides that can be used in the oral dosage forms of the invention include, without limitation, C8-14 alkyl (e.g., octyl-, nonyl-, decyl-, undecyl-, dodecyl-, tridecyl-, or tetradecyl-) ethers of a or β-D-maltoside, -glucoside or -sucroside, alkyl thiomaltosides, such as heptyl, octyl, dodecyl-, tridecyl-, and tetradecyl-β-D-thiomaltoside; alkyl thioglucosides, such as heptyl- or octyl 1-thio α- or β-D-glucopyranoside; alkyl thiosucroses; and alkyl maltotriosides. For example, the echinocandin class compound can be formulated with octyl maltoside, dodecyl maltoside, tridecyl maltoside, or tetradecyl maltoside. Alkyl glucosides that can be used in the oral dosage forms of the invention include, without limitation, C8-14 alkyl (e.g., octyl-, nonyl-, decyl-, undecyl-, dodecyl-, tridecyl-, or tetradecyl-) ethers of glucoside, such as dodecyl glucoside or decyl glucoside.

As used herein, “ester saccharide” refers to sugar esters of a hydrophobic alkyl group (e.g., typically from 8 to 24 carbon atoms in length). Ester saccharides include ester glycosides and ester glucosides. In particular embodiments, the cefepime is formulated with a C8-14 alkyl ester of a sugar. Ester glycosides that can be used in the oral dosage forms of the invention include, without limitation, C8-14 alkyl (e.g., octyl-, nonyl-, decyl-, undecyl-, dodecyl-, tridecyl-, or tetradecyl-) esters of α or β-D-maltoside, -glucoside or -sucroside. For example, the echinocandin class compound can be formulated with sucrose mono-dodecanoate, sucrose mono-tridecanoate, or sucrose mono-tetradecanoate. Ester glucosides that can be used in the oral dosage forms of the invention include, without limitation, C8-14 alkyl (e.g., octyl-, nonyl-, decyl-, undecyl-, dodecyl-, tridecyl-, or tetradecyl-) esters of glucoside, such as glucose dodecanoate or glucose decanoate.

### BRIEF DESCRIPTION OF THE DRAWINGS

FIG. 1 is a graph that shows the ability of huAb1 and muAb1 to block binding of latent TGFβ (L-TGFβ) to αvβ8 as measured by in a cell-based competition assay using LN229 cells which express αvβ8 on their cell surface.

FIG. 2A is a schematic of the treatment protocol associated with the data of FIG. 2B and FIG. 2C. MC38 tumors were grown in mice to about 80 mm³. muAb1 or a combination of anti-mouse PD-1 mAb and muAb1 were administered at 10 mg/kg, IP on days 0, 3 and 7.

FIG. 2B is a graph that shows that αVβ8-blockade with muAb1 inhibits tumor growth and augments the efficacy of PD-1 immunotherapy. Tumor growth curves and tumor growth inhibition (TGI,%) are provided.

FIG. 2C is a graph that shows that αVβ8-blockade with muAb1 inhibits tumor growth and augments the efficacy of PD-1 immunotherapy. Change in tumor volume compared to baseline in individual mice is provided. Numbers refer to the number of mice in each group that had smaller tumors following treatment.

FIG. 3A is a schematic of the treatment protocol associated with the data of FIG. 3B and FIG. 3C. MC38 tumors were grown in mice to about 200 mm³. 10 mg/kg muAb1 was dosed by intraperitoneal injection on days 0, 3 and 7. 10 mg/kg anti-mouse PD-1 mAb was dosed by intraperitoneal injection on days 0 and 7.

FIG. 3B is a graph that shows that αVβ8-blockade with muAb1 inhibits tumor growth and augments the efficacy of PD-1 immunotherapy. Tumor growth curves and tumor growth inhibition (TGI,%) are provided.

FIG. 3C is a graph that shows that αVβ8-blockade with muAb1 inhibits tumor growth and augments the efficacy of PD-1 immunotherapy. Change in tumor volume compared to baseline in individual mice is provided. Numbers refer to the number of mice in each group that had smaller tumors following treatment.

FIGS. 4A-4D are a series of graphs showing that tumor reduction induced by muAb1 and its combination with an anti-PD-1 antibody is related to CD8+ T cell infiltration and proliferation within the tumor microenvironment (TME). Tumor size shrinkage (FIG. 4A) is correlated to CD8+ T-cell infiltration (FIG. 4B) and increased proliferation (FIG. 4C) and activation (FIG. 4D) of these CD8+ T cells within the TME.

FIG. 5A is a schematic of the treatment protocol associated with the data of FIG. 5B and FIG. 5C. Long-term survived tumor-free mice after muAb1 and anti-PD-1 combination treatment were re-challenged by subcutaneous injection with MC38 cells 52 days after original tumor implantation.

FIG. 5B is a graph showing that combination treatment of an anti-αvβ8 antibody and an anti-PD-1 antibody elicits long-term antitumor immunity.

FIG. 5C is a graph showing that tumors did not grow in mice previously treated with the muAb1 and anti-PD-1 mAb combination therapy.

FIG. 6 is a graph showing the biodistribution of muAb1 in plasma and tumor at 24 and 72 hr post intraperitoneal injection of 10 mg/kg muAb1.

FIG. 7A is a schematic of the treatment protocol associated with the data of FIG. 8B. EMT-6 tumors were grown in mice to about 60 mm³. muAb1 or a combination of anti-mouse PD-1 mAb and muAb1 were administered at 10 mg/kg, IP on days 0, 3 and 7.

FIG. 7B is a graph showing antitumor activity of muAb1 as a single agent and that muAb1 reverses resistance to PD-1 therapy in an immune excluded EMT-6 breast tumor model.

FIG. 8A is a schematic of the treatment protocol associated with the data of FIGS. 8B and 8C. EMT-6 tumors were grown in mice to about 230 mm³. Mice were treated by intraperitoneal injection with 10 mg/kg anti-mouse PD-1 mAb on days 0 and 7, and 30 mg/kg muAb1 on days 0, 3 and 7.

FIG. 8B is a graph showing antitumor activity of muAb1 as a single agent and that muAb1 reverses resistance to PD-1 therapy in an immune excluded EMT-6 breast tumor model.

FIGS. 8C-8J are a series of graphs showing that administration of muAb1 and its combination with an anti-PD-1 overcomes immune exclusion in EMT-6 tumors. Tumor size shrinkage (FIG. 8C) is correlated to CD3+ T-cell infiltration (FIG. 8D), CD4+ T-cell infiltration (FIG. 8E), CD8+ T-cell infiltration (FIG. 8F), NK cell infiltration (FIG. 8G), an increase in the M1/M2 ratio (FIG. 8H), an increase in Ki67⁺CD4+ T-cells (FIG. 8I), and an increase in Ki67⁺CD4+ T-cells (FIG. 8J).

FIG. 9A is a schematic of the treatment protocol associated with the data of FIG. 9B. 4T1 tumors were grown in mice to about 50 mm³. muAb1 or a combination of anti-mouse PD-1 mAb and muAb1 were administered at 10 mg/kg, IP on days 0, 3 and 7.

FIG. 9B is a graph showing that muAb1 reverses resistance to PD-1 therapy in an immune desert 4T1 breast tumor model.

FIG. 10A is a graph showing body weight changes of different treatment groups in female C57BL/6 mice bearing xID8 tumors. Data points represent group mean body weight. Error bars represent standard error of the mean (SEM).

FIG. 10B is a graph showing relative change of body weights (%). Percentage change of the body weight of different treatment groups in female C57BL/6 mice bearing xID8 tumors is shown. Body weight change was calculated based on animal weight on the first day of dosing (DO). Data points represent percent group mean change in body weight. Error bars represent standard error of the mean (SEM).

FIG. 11 is a graph showing tumor growth curves of different treatment groups of C57BL/6 mice bearing xID8 tumors. Data points represent group mean, error bars represent standard error of the mean (SEM).

FIG. 12 is a graph showing body weight changes of different treatment groups in female C57BL/6 mice bearing GL261 established tumors in the efficacy study. Data points represent group mean body weight. Error bars represent standard error of the mean (SEM).

FIG. 13 is a graph showing percentage change of the body weight of different treatment groups in female C57BL/6 mice bearing GL261 established tumors in the efficacy study. Body weight change was calculated based on animal weight on the first day of dosing (DO). Data points represent percent group mean change in body weight. Error bars represent standard error of the mean (SEM).

FIG. 14 is a graph showing the tumor growth curves of different treatment groups of C57BL/6 mice bearing GL261 established tumors in the efficacy study. Data points represent group mean, error bars represent standard error of the mean (SEM).

FIG. 15 is a graph showing the survival curve of C57BL/6 mice bearing GL261 established tumors in the efficacy study. Data points represent the mouse survival curves of different treatment groups of C57BL/6 mice bearing GL261 established tumors in the efficacy study. The animals bearing tumor size more than 2,000 mm³ were euthanized.

FIG. 16 is a graph showing body weight changes of different treatment groups in female C57BL/6 mice bearing GL261 established tumors in the pharmacodynamics (PD) study. Data points represent group mean body weight. Error bars represent standard error of the mean (SEM).

FIG. 17 is a graph showing percentage change of the body weight of different treatment groups in female C57BL/6 mice bearing GL261 established tumors in the PD study. Body weight change was calculated based on animal weight on the first day of dosing (DO). Data points represent percent group mean change in body weight. Error bars represent standard error of the mean (SEM).

FIG. 18 is a graph showing the tumor growth curves of different treatment groups of C57BL/6 mice bearing GL261 established tumors in the PD study. Data points represent group mean, error bars represent standard error of the mean (SEM).

FIG. 19 is a graph showing the tumor lysate TGF-β1 levels collected from GL261 tumor-bearing C57BL/6 mice. Data points represent group mean, error bars represent standard error of mean (SEM).

FIG. 20 is a graph showing serum TGF-β1 levels collected from GL261 tumor-bearing C57BL/6 mice. Data points represent group mean, error bars represent standard error of mean (SEM).

FIG. 21 is a graph showing VEGF levels in tumor lysate collected from GL261 tumor-bearing C57BL/6 mice. Data points represent group mean, error bars represent standard error of mean (SEM).

FIG. 22 is a graph showing VEGF levels in serum collected from GL261 tumor-bearing C57BL/6 mice. Data points represent group mean, error bars represent standard error of mean (SEM).

FIGS. 23A-23C are a series of graphs showing tumor p/tSmad2, p/tSmad3 and Sox4 levels. The change of p/tSmad2, p/tSmad3 and Sox4 in tumor lysate collected from GL261 tumor-bearing C57BL/6 mice is shown. Immunoblotting images (A) and protein greyscale analysis (B and C) are provided. Data points represent group mean, error bars represent standard error of mean (SEM).

FIGS. 24A-24B are a series of graphs showing tumor Itgb6 (A) and Itgb8 (B) transcription levels. The change of transcription level of Itgb6 and Itgb8 in tumor collected from GL261 tumor-bearing C57BL/6 mice is shown. Data points represent group mean, error bars represent standard error of mean (SEM).

FIG. 25 is a graph showing tumor volume of MC38 model on Day 10. The tumor volume of different treatment groups in female C57BL/6 mice bearing MC38 tumors is shown. Data points represent group mean, and error bars represent standard error of the mean (SEM).

FIG. 26 is a graph showing the percentage of CD45+ and tumor cells in live cells in tumor for panel 1.

FIGS. 27A-27C are a series of graphs showing the percentage of immune cell subpopulations in CD45+ live cells in tumor for panel 1.

FIGS. 28A-28B are a series of graphs showing ratios of CD4Teff/Treg, CD8T/Treg and M1/M2 in tumor for panel 1.

FIGS. 29A-29B are a series of graphs showing the positive rate and MFI of Granzyme B in immune cell subpopulations in tumor for panel 1.

FIGS. 30A-30B are a series of graphs showing the positive rate and MFI of PD-1 in immune cell subpopulations in tumor for panel 1.

FIGS. 31A-31B are a series of graphs showing the positive rate and MFI of TIM-3 in immune cell subpopulations in tumor for panel 1.

FIGS. 32A-32B are a series of graphs showing the positive rate and MFI of Ki67 in immune cell subpopulations in tumor for panel 1.

FIGS. 33A-33B are a series of graph showing the positive rate and MFI of TCF-1 in immune cell subpopulations in tumor for panel 1.

FIGS. 34A-34B are a series of graphs showing the percentage of MuLV p15E specific CD8T in CD45+live cells and CD8T in stimulated tumor for Panel 2.

FIGS. 35A-35B are a series of graphs showing the positive rate and MFI of Granzyme B in immune cell subpopulations in stimulated tumor for panel 2.

FIGS. 36A-36B are a series of graphs showing the positive rate and MFI of IFN-g in immune cell subpopulations in stimulated tumor for panel 2.

FIGS. 37A-37B are a series of graphs showing the positive rate and MFI of IL-2 in immune cell subpopulations in stimulated tumor for panel 2.

FIGS. 38A-38B are a series of graphs showing the positive rate and MFI of TNF-α in immune cell subpopulations in stimulated tumor for panel 2.

FIGS. 39A-39B are a series of graphs showing the positive rate and MFI of Ki67 in immune cell subpopulations in stimulated tumor for panel 2.

FIGS. 40A-40C are a series of graphs showing the percentage of immune cell subpopulations in CD45+ live cells in blood for Panel 3.

FIG. 41 is a graph showing ratios of CD4Teff/Treg and CD8T/Treg in blood for panel 3.

FIGS. 42A-42B are a series of graphs showing the positive rate and MFI of Granzyme B in immune cell subpopulations in blood for panel 3.

FIGS. 43A-43B are a series of graphs showing the positive rate and MFI of PD-1 in immune cell subpopulations in blood for panel 3.

FIGS. 44A-44B are a series of graphs showing the positive rate and MFI of Ki67 in immune cell subpopulations in blood for panel 3.

### DETAILED DESCRIPTION

The present disclosure provides antibodies that bind specifically to integrin αvβ8. The disclosure also provides methods for enhancing an immune response in a subject. The disclosure also provides methods for treating or preventing αvβ8-associated disorders. In some embodiments, the antibodies are suitable for use (e.g., therapeutic use) in a human subject.

Antibodies of the present disclosure may have improved binding to αvβ8, improved inhibition of TGF-β (e.g., TGF-β induced signaling), or reduced immunogenicity (e.g., in humans), as compared to anti-αvβ8 antibodies previously described. The disclosed antibodies may have improved pharmacokinetic properties (e.g., half-life), greater potency, or greater efficacy in translational models relevant to cancer treatment and targeted immune cell activation, as compared to anti-αvβ8 antibodies previously described. The disclosed antibodies may also confer greater specificity and selectivity towards avb8 compared to other cell-surface targets, as compared to anti-αvβ8 antibodies previously described. The disclosed antibodies or antigen-binding fragments thereof may have greater thermal or chemical stability, increased expression (e.g., from a cell line or mammal, such as a human or non-human mammal), reduced aggregation (e.g., in a buffer or excipient), greater resistance to post-translational modification such as deamidation, reduced susceptibility to side chain oxidation (e.g., on one or more methionine or tryptophan residues), or reduced susceptibility to N-linked glycation.

### I. Anti-αvβ8 Antibodies

The disclosure provides antibodies that bind specifically to integrin αvβ8. Provided herein are antibodies that bind human (and in some embodiments other mammalian, e.g., such as mouse, guinea pig, pig, and rabbit) integrin αvβ8. In some embodiments, the antibodies specifically bind human integrin αvβ8 and block binding of a ligand to human integrin αvβ8. Exemplary ligands can include, for example, TGF-β and the latency associate peptide (LAP) of TGF-β.

The ability of an antibody to block αvβ8 integrin binding of a ligand can be determined by inhibition of binding of a soluble form of αvβ8 or a full-length form of αvβ8 expressed on the surface of cells to immobilized latent-TGF-β or a portion thereof containing the sequence RGDL. See, e.g., Ozawa, A, et al. J Biol Chem. 291(22):11551-65 (2016).

In particular, the disclosure features anti-αvβ8 antibodies with a light chain variable domain (VL) comprising an amino acid sequence of SEQ ID NO: 1 (DIVMTQSPDSLAVSLGERATINCKSSQSLLHSRSRKNYLAWYQQKPGQPPKLLIYWASTRESGVPDRFS GSGSGTDFTLTISSLQAEDVAVYYCKQSYNLLSFGQGTKLEIK) and a heavy chain variable domain (VH) comprising an amino acid sequence of SEQ ID NO: 2 (QIQLVQSGSELKKPGASVKVSCKASGYTFTKYSMHWVRQAPGQGLEWMARINTETGNPTYADGFRGR FVVSLDTSVSTAYLQISSLKAEDTAVYYCAIFYYGRDTWGQGTTVTVSS).

In some embodiments, the antibody comprises a light chain amino acid sequence of SEQ ID NO: 3 (DIVMTQSPDSLAVSLGERATINCKSSQSLLHSRSRKNYLAWYQQKPGQPPKLLIYWASTRESGVPDRFS GSGSGTDFTLTISSLQAEDVAVYYCKQSYNLLSFGQGTKLEIKRTVAAPSVFIFPPSDEQLKSGTASVVCL LNNFYPREAKVQWKVDNALQSGNSQESVTEQDSKDSTYSLSSTLTLSKADYEKHKVYACEVTHQGLSSP VTKSFNRGEC) and a heavy chain amino acid sequence of SEQ ID NO: 4 (QIQLVQSGSELKKPGASVKVSCKASGYTFTKYSMHWVRQAPGQGLEWMARINTETGNPTYADGFRGR FVVSLDTSVSTAYLQISSLKAEDTAVYYCAIFYYGRDTWGQGTTVTVSSASTKGPSVFPLAPCSRSTSEST AALGCLVKDYFPEPVTVSWNSGALTSGVHTFPAVLQSSGLYSLSSVVTVPSSSLGTKTYTCNVDHKPSNT KVDKRVESKYGPPCPPCPAPEFLGGPSVFLFPPKPKDTLMISRTPEVTCVVVDVSQEDPEVQFNWYVDG VEVHNAKTKPREEQFNSTYRVVSVLTVLHQDWLNGKEYKCKVSNKGLPSSIEKTISKAKGQPREPQVYTL PPSQEEMTKNQVSLTCLVKGFYPSDIAVEWESNGQPENNYKTTPPVLDSDGSFFLYSRLTVDKSRWQE GNVFSCSVMHEALHNHYTQKSLSLSLGK).

### Production and Characterization of Antibodies

For preparation and use of suitable antibodies as described herein, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. Nos. 4,946,778, 4,816,567) can be adapted to produce antibodies to polypeptides of this disclosure. Also, transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody includes both a VH and VL region, the VH and VL regions may be expressed using a single vector, e.g., in a di-cistronic expression unit, or under the control of different promoters. In other embodiments, the VH and VL region may be expressed using separate vectors. A VH or VL region as described herein may optionally include a methionine at the N-terminus.

In some cases, the antibody can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227, 2007). The antibody can also be labeled, or conjugated to a therapeutic agent as described below.

The specificity of antibody binding can be defined in terms of the comparative dissociation constants (Kd) of the antibody for the target (e.g., αvβ8) as compared to the dissociation constant with respect to the antibody and other materials in the environment or unrelated molecules in general. Typically, the Kd for the antibody with respect to the unrelated material will be at least 2-fold, 3-fold, 4-fold, 5-fold, 10-fold, 20-fold, 50-fold, 100-fold, 200-fold or higher than Kd with respect to the target.

The desired affinity for an antibody, e.g., high (pM to low nM), medium (low nM to 100 nM), or low (about 100 nM or higher), may differ depending upon whether it is being used as a diagnostic or therapeutic. For example, an antibody with medium affinity may be more successful in localizing to desired tissue as compared to one with a high affinity. Thus, antibodies having different affinities can be used for diagnostic and therapeutic applications.

A targeting moiety will typically bind with a Kd of less than about 1000 nM, e.g., less than 250, 100, 50, 20 or lower nM. In some embodiments, the Kd of the affinity agent is less than 15, 10, 5, or 1 nM. In some embodiments, the Kd is 1-100 nM, 0.1-50 nM, 0.1-10 nM, or 1-20 nM. The value of the dissociation constant (Kd) can be determined by well-known methods and can be computed even for complex mixtures by methods as disclosed, e.g., in Caceci et al., Byte (1984) 9:340-362.

Affinity of an antibody, or any targeting agent, for a target can be determined according to methods known in the art, e.g., as reviewed in Ernst et al. Determination of Equilibrium Dissociation Constants, Therapeutic Monoclonal Antibodies (Wiley & Sons ed. 2009).

Quantitative ELISA, and similar array-based affinity methods can be used. ELISA (Enzyme linked immunosorbent signaling assay) is an antibody-based method. In some cases, an antibody specific for target of interest is affixed to a substrate, and contacted with a sample suspected of containing the target. The surface is then washed to remove unbound substances. Target binding can be detected in a variety of ways, e.g., using a second step with a labeled antibody, direct labeling of the target, or labeling of the primary antibody with a label that is detectable upon antigen binding. In some cases, the antigen is affixed to the substrate (e.g., using a substrate with high affinity for proteins, or a Strepavidin-biotin interaction) and detected using a labeled antibody (or other targeting moiety). Several permutations of the original ELISA methods have been developed and are known in the art (see Lequin (2005) Clin. Chem. 51:2415-18 for a review). Such methods may also include solution phase assays such as the AlphaScreen® assay wherein Histidine tagged or biotin labeled human avb8 Heterodimer is mixed with increasing amounts of antibody. Then Anti-IgG4 AlphaLISA Acceptor beads are added and then following an incubation, Nickel Chelate Alpha Donor beads (for a Histidine tagged integrin) or Streptavidin Alpha Donor beads (for a biotinylated integrin) are added and the Alpha Counts are read on a compatible plate reader.

The Kd, Kon, and Koff can also be determined using surface plasmon resonance (SPR), e.g., as measured by using a Biacore T100 system or using kinetic exclusion assays (e.g., KinExA®). SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given frequency is shined on the surface, the changes to the optical reflectivity of the gold indicate binding, and the kinetics of binding. Kinetic exclusion assays may also be used to affinity, especially for slow dissociating high affinity antibodies. This technique is described in, e.g., Darling et al., Assay and Drug Development Technologies Vol. 2, number 6 647-657 (2004).

Binding affinity can also be determined by anchoring a biotinylated interactant to a streptavidin (SA) sensor chip. The other interactant is then contacted with the chip and detected, e.g., as described in Abdessamad et al. (2002) Nuc. Acids Res. 30:e45.

The invention also features polynucleotides (e.g., DNA or RNA) encoding the antibodies described herein, e.g., polynucleotides, expression cassettes (e.g., a promoter linked to a coding sequence), or expression vectors encoding heavy or light chain variable regions or segments including the complementary determining regions as described herein. In some embodiments, the polynucleotide sequence is optimized for expression, e.g., optimized for mammalian expression or optimized for expression in a particular cell type. In some embodiments, the vector is an expression vector (e.g., a eukaryotic expression vector). In some embodiments, the vector is a viral vector (e.g., adenovirus (Ad), retrovirus, poxvirus, adeno-associated virus, baculovirus, or herpes simplex virus). Also provided are host cells comprising any of the vectors described herein.

### II. Immune Checkpoint Inhibitors

An immune checkpoint inhibitor may be administered or formulated in combination with an anti-αVβ8 antibody described herein.

Immune checkpoints refer to a plethora of inhibitory pathways hardwired into the immune system, which, under normal physiological conditions are crucial for maintaining self-tolerance and modulating the duration and amplitude of physiological immune responses in peripheral tissues to minimize collateral tissue damage in response to pathogenic infection. However, the expression of immune checkpoint proteins is often dysregulated by tumors as an important immune resistance and escape mechanism.

Because many of the immune checkpoints are initiated by ligand-receptor interactions, they can be readily blocked by antibodies or modulated by recombinant forms of ligands or receptors. Thus, inhibition of these pathways has been used to activate therapeutic anti-tumor immunity. For example, cytotoxic T-lymphocyte-associated antigen 4 (CTLA-4) antibodies were the first of this class of immunotherapeutics to achieve US Food and Drug Administration (FDA) approval. Preliminary clinical findings with inhibitors of additional immune-checkpoint proteins, such as programmed cell death protein 1 (PD-1), indicate broad and diverse opportunities to enhance anti-tumor immunity with the potential to produce durable clinical responses.

T cell activation through blockade of immune checkpoints has been a major focus of efforts to therapeutically manipulate endogenous anti-tumor immunity, owing to the capacity of T cells for the selective recognition of peptides derived from proteins in all cellular compartments; their capacity to directly recognize and kill antigen-expressing cells (by CD8⁺ effector T cells; also known as cytotoxic T lymphocytes (CTLs)); and their ability to orchestrate diverse immune responses (by CD4+ helper T cells), which integrate adaptive and innate effector mechanisms. Thus, agonists of co-stimulatory receptors or antagonists of inhibitory signals, both of which result in the amplification of antigen-specific T cell responses, are currently agents of interest in clinical testing.

TABLE 1
Non-limiting list of immune checkpoint targets.

| Target | Biological Function |
|---|---|
| CTLA-4 | Inhibitory Receptor |
| PD-1 | Inhibitory Receptor |
| PD-L1 | Ligand for PD-1 |
| LAG-3 | Inhibitory Receptor |
| B7.1 | Costimulatory Molecule |
| B7-H3 | Inhibitory Ligand |
| B7-H4 | Inhibitory Ligand |
| TIM3 | Inhibitory Receptor |
| VISTA | Inhibitory Receptor |
| CD137 | Costimulatory Molecule |
| OX-40 | Costimulatory Receptor |
| CD40 | Costimulatory Molecule |
| CD27 | Costimulatory Receptor |
| CCR4 | Costimulatory Receptor |
| GITR | Costimulatory Receptor |
| NKG2D | Activating Receptor |
| KIR | Costimulatory Receptor |

CTLA4, cytotoxic T-lymphocyte-associated antigen 4; LAG3, lymphocyte activation gene 3; PD-1, programmed cell death protein 1; PD-L1, PD-1 ligand; TIM3, T cell membrane protein 3; VISTA, V-domain immunoglobulin (Ig)-containing suppressor of T-cell activation; KIR, killer IgG-like receptor.

ICIs approved or in development include, but are not limited to, YERVOY® (ipilimumab), OPDIVO® (nivolumab), KEYTRUDA® (pembrolizumab), tremelimumab, galiximab, MDX-1106, BMS-936558, MED14736, MPDL3280A, MED16469, BMS-986016, BMS-663513, PF-05082566, IPH2101, KW-0761, CDX-1127, CP-870, CP-893, GSK2831781, MS1B0010718C, MK3475, CT-Oil, AMP-224, MDX-1105, IMP321, and MGA271, as well as numerous other antibodies or fusion proteins directed to the immune checkpoint proteins noted in Table 1. Common immune checkpoint proteins that may be targeted by ICIs include, but are not limited to B7.1, B7-H3, LAG3, CD137, KIR, CCR4, CD27, OX40, GITR, CD40, CTLA4, PD-1, and PD-L1.

In some embodiments the ICI is a PD-(L)1 inhibitor, which is a molecule that inhibits the interaction of a PD-1 axis binding partner with either one or more of its binding partner, so as to remove T-cell dysfunction resulting from signaling on the PD-1 signaling axis, with a result being to restore or enhance T-cell function (e.g., proliferation, cytokine production, target cell killing). A PD-(L)1 inhibitor may be an anti-PD-1 antibody (e.g., cemiplimab, nivolumab, pembrolizumab, dostarlimab, toripalimab, tislelizumab, or retifanlimab) or an anti-PD-L1 antibody (e.g., atezolizumab, avelumab, or durvalumab).

In some embodiments, the ICI therapy is selected from one or more of anti-PD-1, anti-PD-L1, anti-CTLA-4, anti-LAG3, anti-B7.1, anti-B7H3, anti-B7H4, anti-TIM3, anti-VISTA, anti-CD137, anti-OX40, anti-CD40, anti-CD27, anti-CCR4, anti-GITR, anti-NKG2D, and anti-KIR. In some embodiments, the ICI therapy is an antibody (e.g., a monoclonal antibody selective for any of the targets in Table 1). In some embodiments the ICI is an anti-PD-1 antibody.

### III. CTLA-4 Inhibitors

A CTLA-4 inhibitor may be administered in combination with an anti-αVβ8 antibody described herein.

A CTLA-4 inhibitor is a compound, such as an antibody, capable of inhibiting the activity of the protein that in humans is encoded by the CTLA4 gene. CTLA-4 inhibitors include tremilimumab and ipilimumab. Ipilimumab is a recombinant human IgG1 kappa monoclonal antibody that binds to the cytotoxic T-lymphocyte-associated protein 4 (CTLA-4). CTLA-4 is a negative regulator of T-cell activity. By binding to CTLA-4, ipilimumab blocks the interaction of CTLA-4 with its ligands, CD80/CD86. Blockade of CTLA-4 has been shown to augment T-cell activation and proliferation, including the activation and proliferation of tumor infiltrating T-effector cells. Inhibition of CTLA-4 signaling can also reduce T-regulatory cell function, which may contribute to a general increase in T cell responsiveness, including the anti-tumor immune response.

### IV. LAG-3 Inhibitors

A LAG-3 inhibitor may be administered in combination with an anti-αVβ8 antibody described herein.

A LAG-3 inhibitor is a compound, such as an antibody, capable of inhibiting the activity of the protein that in humans is encoded by the LAG-3 gene. LAG-3 inhibitors may include relatlimab.

### V. Angiogenesis Inhibitors

An angiogenesis inhibitor may be administered in combination with an anti-αVβ8 antibody described herein.

An angiogenesis inhibitor refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. The angiogenesis inhibitor may include those agents that bind and block the angiogenic activity of the angiogenic factor or its receptor. For example, an angiogenesis inhibitor is an antibody or other antagonist to an angiogenic agent, e.g., antibodies to VEGF-A or the VEGF-A receptor (e.g., KDR receptor or Flt-1 receptor), anti-PDGFR inhibitors such as GLEEVEC™ (imatinib mesylate). Angiogenesis inhibitors also include native angiogenesis inhibitors, e.g., angiostatin, endostatin, etc. See, for example, Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) and Sato Int. J. Clin. Oncol., 8:200-206 (2003).

In some embodiments, the angiogenesis inhibitor may be axitinib, tivozanib, vandetanib, nintedanib, sunitinib, or sorafenib.

In some embodiments, the angiogenesis inhibitor may be an anti-VEGF antibody, which is an antibody that binds to VEGF. In some embodiments the anti-VEGF antibody is bevacizumab, also known as “rhuMAb VEGF,” “BV,” or “AVASTIN®.” Bevacizumab is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 Daltons and is glycosylated. Bevacizumab and other humanized anti-VEGF antibodies are further described in U.S. Pat. No. 6,884,879, issued Feb. 26, 2005, the entire disclosure of which is expressly incorporated herein by reference. Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al. (Journal of Immunological Methods 288:149-164, 2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183, and Q89.

In some embodiments, the angiogenesis inhibitor may be an anti-VEGF receptor. Anti-VEGF receptor antibodies may include antibodies that bind VEGFR-2 (e.g., ramucirumab) and antibodies that bind VEGFR-1 (e.g., IMC-18F1).

In some embodiments, the angiogenesis inhibitor may be an anti-VEGF trap. Anti-VEGF traps may include sequences from the native receptors VEGFR1 and VEGFR2. In some embodiments, the anti-VEGF trap may be aflibercept.

### VI. Chemotherapeutic Agents

A chemotherapeutic agent may be administered in combination with an anti-αVβ8 antibody described herein.

A chemotherapeutic agent refers to chemical compounds useful in the treatment of cancer. Examples of chemotherapeutic agents include alkylating agents, intercalating agents, antimicrotubule agents, antimitotics, antimetabolites, antiproliferative agents, antibiotics, immunomodulatory agents, anti-inflammatories, kinases inhibitors, antivascular agents, oestrogenic and androgenic hormones. In some embodiments, the chemotherapeutic agent is platinum chemotherapy (e.g., oxaliplatin, carboplatin, or cisplatin), trastuzumab, fluoropyrimidine, gemcitabine, irinotecan, 5-fluorouracil, or a taxane (e.g., docetaxel or paclitaxel).

### VII. Immune-Priming Stereotactic Body Radiation Therapy

Immune-priming stereotactic body radiation therapy (SBRT) may be administered in combination with an anti-αVβ8 antibody described herein.

Immune-priming SBRT refers to highly potent ablative doses of radiation which stimulate expression of MHC class 1 molecules for enhanced tumor recognition, release of tumor damage-associated molecular patterns (DAMPs) which may lead to maturation of dendritic cells, stimulation of cytotoxic T-cell activity via dendritic-cell antigen presentation, and enhanced anti-tumor immunomodulation. In some embodiments, the immune-priming SBRT is administered at a dose of 8 Gy on Day 1, Day 3, and Day 5 of the one or more dosing cycles.

### VIII. Pharmaceutical Compositions

Also provided are pharmaceutical compositions including the present anti-αvβ8 antibodies, which can be formulated together with a pharmaceutically acceptable carrier. The compositions can additionally contain other therapeutic agents that are suitable for treating or preventing a given disorder. Pharmaceutically carriers can enhance or stabilize the composition, or to facilitate preparation of the composition. Pharmaceutically acceptable carriers include solvents, dispersion media, coatings, antibacterial and antifungal agents, isotonic and absorption delaying agents, and the like that are physiologically compatible.

In some embodiments, the composition is sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the disclosure can be prepared in accordance with methods well known and routinely practiced in the art. Pharmaceutically acceptable carriers are determined in part by the particular composition being administered, as well as by the particular method used to administer the composition. Accordingly, there is a wide variety of suitable formulations of pharmaceutical compositions of the present disclosure. Applicable methods for formulating the antibodies and determining appropriate dosing and scheduling can be found, for example, in Remington: The Science and Practice of Pharmacy, 21^{st} Ed., University of the Sciences in Philadelphia, Eds., Lippincott Williams & Wilkins (2005); and in Martindale: The Complete Drug Reference, Sweetman, 2005, London: Pharmaceutical Press., and in Martindale, Martindale: The Extra Pharmacopoeia, 31^{st} Edition., 1996, Amer Pharmaceutical Assn, and Sustained and Controlled Release Drug Delivery Systems, J. R. Robinson, ed., Marcel Dekker, Inc., New York, 1978, each of which are hereby incorporated herein by reference. Pharmaceutical compositions are preferably manufactured under GMP conditions. Typically, a therapeutically effective dose or efficacious dose of the anti-αvβ8 antibody is employed in the pharmaceutical compositions of the disclosure. The anti-αvβ8 antibodies are formulated into pharmaceutically acceptable dosage forms by conventional methods known to those of skill in the art.

In some embodiments, the pharmacological compositions include a mixture of the anti-αvβ8 antibody and a second pharmacological agent. Without intending to limit the disclosure, it is noted that the thymic stromal lymphopoietin (TSLP) is an inducer of viral clearance in a mouse model of acute and chronic HBV and thus is useful to combine TSLP with an αvβ8 antibody for anti-viral treatments. Moreover, OX40 agonists are effective in stimulating an immune response to HBV in combination with an αvβ8 antibody.

As an alternative to mixing the anti-αvβ8 antibody and second pharmacological agent in a pharmacological composition, the anti-αvβ8 antibody and second pharmacological agent can be separately administered to the human in need thereof within a time frame (e.g., within 3, 2, or 1 day or within 24, 13, 6, or 3 hours of each other).

In some embodiments, the pharmaceutical composition is an aqueous formulation having a pH of between 5.0 and 7.5 (e.g., a pH of 5.5 0.5, 6.0 0.5, 6.5±0.5, or 7.0±0.5) and the antibody is present at a concentration of between 20 mg/mL and 40 mg/mL (e.g., a concentration of 25±5 mg/mL, 30±5 mg/mL, or 35±5 mg/mL).

In some embodiments, the pharmaceutical composition comprises 10 to 30 mM (e.g., 15±5 mM, 20±5 mM, or 25±5 mM) phosphate buffer or histidine buffer.

In some embodiments, the pharmaceutical composition comprises 0.01% (w/w) to 0.1% (w/w) (e.g., 0.02±0.01%, 0.04±0.02%, 0.06±0.02%, or 0.08±0.02%) of a nonionic surfactant. In some embodiments, the nonionic surfactant is selected from polysorbate surfactants, polyglycolized glycerides, alkyl saccharides, and ester saccharides. In some embodiments, the nonionic surfactant is polyoxyethylene 20 sorbitan monolaurate.

In some embodiments, the pharmaceutical composition comprises from 40 to 60 mM (e.g., 45±5 mM, 50±5 mM or 55±5 mM) arginine or glutamate.

In some embodiments, the pharmaceutical composition comprises a nonionic tonicity agent. In some embodiments, the nonionic tonicity agent is selected from sucrose, mannitol, sorbitol, lactose, dextrose, trehalose, and glycerol. In some embodiments, the nonionic tonicity agent is sucrose, and the pharmaceutical composition comprises from 4% (w/w) to 7.5% (w/w) sucrose (e.g., 4.5±0.5%, 5.0±0.5%, 5.5±0.5%, 6.0±0.5%, 6.5±0.5%, or 7.0±0.5%).

In some embodiments, the pharmaceutical composition comprises less than 5 mM sodium chloride, or is free of sodium chloride.

In some embodiments, the antibody is present at a concentration of about 30 mg/mL.

In some embodiments, the pharmaceutical composition comprises 20 mM sodium phosphate, 5% (w/w) sucrose, 50 mM arginine, and 0.02% (w/w) polyoxyethylene 20 sorbitan monolaurate.

In some embodiments, the pharmaceutical composition is formulated at a pH of about 6.5.

In some embodiments, the pharmaceutical composition is formulated in a volume of between 1 to 30 mL (e.g., 5±4 mL, 10±5 mL, 15±5 mL, 20±5 mL, or 25±5 mL). In some embodiments, the pharmaceutical composition is formulated in a volume of about 10 mL.

### IX. Methods of Treatment

Anti-αvβ8 antibodies described herein or a pharmaceutical composition thereof can be used to treat a patient suffering from a disorder associated with αvβ8 and/or to enhance an immune response in a subject.

The anti-αvβ8 antibodies described herein can be used to treat, ameliorate, or prevent cancer. In some embodiments, the antibody enhances the immune response against the cancer, thereby treating the cancer. In some embodiments, the cancer is a solid tumor. In some embodiments, the cancer is a metastatic cancer. In some embodiments, the cancer is a primary cancer. In some embodiments, the cancer is selected from lung cancer, optionally non-small cell lung cancer or small-cell lung cancer; head and neck cancer, optionally head and neck squamous cell carcinoma; renal cell carcinoma, optionally renal clear cell carcinoma or kidney renal papillary cell carcinoma; breast cancer, optionally triple-negative breast cancer; ovarian cancer; pancreatic cancer; brain cancer, optionally gliomas; colorectal cancer; urothelial cancer; bile duct cancer; endometrial cancer; melanoma; cervical cancer; gastric cancer; hepatocellular carcinoma; glioblastoma; or esophageal cancer.

The cancer may be an immune refractory cancer for which a prior immunotherapy, such as an immune checkpoint inhibitor, has been found ineffective or intolerable. For example, a patient having an immune refractory cancer includes a patient who has previously been administered an immunotherapy, such as an immune checkpoint inhibitor, and the immunotherapy has been found ineffective or found not adequately effective to slow or halt the progression of the disease or to alleviate symptoms associated with the progression of the disease. Immune refractory cancers include cancers that have become resistant to or desensitized to treatment with immunotherapy (e.g., the effectiveness of an immunotherapy, such as an immune checkpoint inhibitor, previously administered to the patient is diminished over time). Immune refractory cancers can be identified by methods known to those of skill in the art or by methods described herein. For example, immune refractory cancers may be characterized by low immune cell infiltrate in one or more tumors. Low immune cell infiltrate may include a decrease or absence of lymphocytes; a decrease or absence of tumor-infiltrating lymphocytes (TILs); a decrease or absence of dendritic cells; a decrease or absence of myeloid cells; a decrease or absence of natural killer (NK) cells; a decrease or absence of macrophages; a decrease or absence of T cells; a decrease or absence of CD8+ T cells; a decrease or absence of CD4+ T cells; or a decrease or absence of CD4+/CD8+ T cells. See, e.g., Chen and Mellman, Nature, 541:321 (2017).

The subject may have had disease progression after at least one line of therapy (e.g., an immune checkpoint inhibitor, e.g., a PD-(L)1 inhibitor) or has no other standard therapy of proven clinical benefit currently available. Disease progression refers to a worsening of a disease. In some instances, disease progression is radiographic disease progression, e.g., as defined by growth of existing lesions, new lesions, or recurrence of previously resolved lesions. Disease progression (e.g., radiographic disease progression) can be determined by RECIST v1.1. In some embodiments, disease progression (or lack of disease progression) is confirmed by a confirmatory scan and/or pathology.

The subject may be resistant to treatment with an immune checkpoint inhibitor (e.g., a PD-(L)1 inhibitor). Resistance to treatment refers to a treatment of a disorder with a therapeutic agent, where the therapeutic agent is ineffective or where the therapeutic agent was previously effective and has become less effective over time. Resistance to treatment includes acquired resistance to treatment, which refers to a decrease in the efficacy of a treatment over a period of time where the subject is being administered the therapeutic agent. Acquired resistance to treatment may result from the acquisition of a mutation in a target protein, or in multiple proteins and genetic loci, that renders the treatment ineffective or less effective. Accordingly, resistance to treatment may persist even after cessation of administration of the therapeutic agent. In particular, a cancer may become resistant to treatment with an immune checkpoint inhibitor following treatment with an immune checkpoint inhibitor. Measurement of a decrease in the efficacy of the treatment will depend on the disorder being treated, and such methods are known to those of skill in the art. For example, efficacy of a cancer treatment may be measured by the progression of the disease. An effective treatment may slow or halt the progression of the disease. A cancer that is resistant to treatment with a therapeutic agent, e.g., an immune checkpoint inhibitor, may fail to slow or halt the progression of the disease. Administration of the antibody or pharmaceutical composition may sensitize the cancer to treatment with an immune checkpoint inhibitor.

Administration of the antibody or pharmaceutical composition thereof may transform an immunologically cold tumor into an immunologically hot tumor. A hot tumor refers to a tumor, cancer, or patient having a tumor or cancer that is not immune refractory. Cells having a low cytotoxic T cell count can be characterized as an “immune desert” or “immune excluded.” In some embodiments, cells having a cytotoxic T cell count of less than 1% live cells are considered an immune desert or immune excluded. In some embodiments, cells having a cytotoxic T cell count of less than 0.5% live cells are considered an immune desert or immune excluded. In some embodiments, cells having a cytotoxic T cell count of less than 0.25% live cells are considered an immune desert or immune excluded. It is also recognized that other cell types such as cancer-associated fibroblasts (CAFs) can contribute to immune exclusion by secreting cytokines such as TGFb into the tumor microenvironment and by synthesizing the fibrotic extracellular matrix that underlies desmoplasia, characteristic of many resistant tumors (Grauel et al., Nature Communications 11, 6315 (2020); Dominguez et al., Cancer Discovery 10, 232 (2020). For immune cells as well as CAFs, it is also recognized that the activation state of specific immune cells (defined by markers such as IFNg, Granzyme B, CD25, CD69) and their genetic program further defines the responsiveness of the tumor to immunotherapy (Cogdill et al., British Journal Cancer 117,1 (2017); Mariathasan, S. et al., Nature 554, 544 (2018)) and inherent restraints to anti-tumor immunity.

Without intending to limit the scope of the disclosure, in some embodiments it is believed that antibodies described herein function in part by triggering an increase in MHCII expression in antigen presenting cells.

In some embodiments, the anti-αvβ8 antibodies disclosed herein are administered as a monotherapy.

In some embodiments, the anti-αvβ8 antibodies disclosed herein are administered in combination with one or additional therapies.

The anti-αvβ8 antibodies may be administered to the subject in combination with: (a) a PD-(L)1 inhibitor; (b) a CTLA-4 inhibitor; (c) a LAG-3 inhibitor; (d) an angiogenesis inhibitor; (e) a chemotherapeutic agent; or (f) immune-priming SBRT.

The anti-αvβ8 antibodies may be administered to the subject in combination with: (a) a PD-(L)1 inhibitor, a CTLA-4 inhibitor, a LAG-3 inhibitor, an angiogenesis inhibitor, or a chemotherapeutic agent; and (b) immune-priming SBRT.

The anti-αvβ8 antibodies may be administered to the subject in combination with: (a) immune-priming SBRT, a CTLA-4 inhibitor, a LAG-3 inhibitor, an angiogenesis inhibitor, or a chemotherapeutic agent; and (b) a PD-(L)1 inhibitor.

### X. Routes of Administration and Dosing

In any of the methods of the invention, the anti-αvβ8 antibody or pharmaceutical composition thereof may be administered at a dose of about 1 mg/kg to about 30 mg/kg (e.g., about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, or about 25 mg/kg to about 30 mg/kg).

In any of the methods of the invention, the anti-αvβ8 antibody or pharmaceutical composition thereof may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, the dosing cycles continue until there is a loss of clinical benefit (e.g., confirmed disease progression, drug resistance, death, or unacceptable toxicity).

In some embodiments, the length of each of the one or more dosing cycles is 21 days. In some embodiments, the antibody that binds to integrin αVβ8 or a pharmaceutical composition thereof is administered on Day 1 of each of the one or more dosing cycles. In some embodiments, the antibody that binds to integrin αVβ8 or a pharmaceutical composition thereof is administered on Day 1 and Day 8 of each of the one or more dosing cycles. In some embodiments, the antibody that binds to integrin αVβ8 or a pharmaceutical composition thereof is administered on Day 1, Day 8, and Day 15 of each of the one or more dosing cycles.

In some embodiments, the length of each of the one or more dosing cycles is 28 days. In some embodiments, the antibody that binds to integrin αVβ8 or a pharmaceutical composition thereof is administered on Day 1 and Day 15 of each of the one or more dosing cycles. In some embodiments, the antibody that binds to integrin αVβ8 or a pharmaceutical composition thereof is administered on Day 1, Day 8, Day 15, and Day 22 of each of the one or more dosing cycles. In some embodiments, the antibody that binds to integrin αVβ8 or a pharmaceutical composition thereof is administered on Day 1, Day 8, and Day 15 of each of the one or more dosing cycles.

In some instances, the antibody that binds to integrin αVβ8 or pharmaceutical composition thereof is administered intravenously. In some instances, the antibody or pharmaceutical composition thereof is administered by intravenous infusion over 30±5 minutes.

### XI. Diagnostic Compositions and Applications

Integrin αvβ8 is expressed on fibroblasts, stellate cells, chondrocytes, activated macrophages and subsets of T and B-cells. Integrin αvβ8 is increased in expression in fibroblasts in COPD and pulmonary fibrosis and can be used as a surrogate marker for increased fibroblast cell mass. Thus the presently disclosed antibodies can be broadly applicable to bioimaging strategies to detect fibroinflammatory processes, e.g., in any disorder described herein. The presently described therapeutic and diagnostic antibodies can be applied to, for example, inflammatory bowel disease (IBD), chronic obstructive pulmonary disease (COPD), asthma, arthritis, a hepatic fibroinflammatory disorder, alcohol induced liver injury, non-alcoholic steatohepatitis (NASH), viral hepatitis, and primary biliary cirrhosis (PBC), graft rejection after liver transplantation, autoimmune hepatitis, an autoimmune disorder, lupus erythematosus, scleroderma, dermatomyositis, bullous pemphigoid, pemphigus vulgaris, a pulmonary fibrotic disorder, an inflammatory brain autoimmune disease, multiple sclerosis, a demyelinating disease, neuroinflammation, kidney disease, glomerulonephritis, hepatocellular carcinoma (HCC), adenocarcinoma, squamous carcinoma, glioma, melanoma, lung, prostate, ovarian, uterine, or breast cancer.

Anti-αvβ8 antibodies described herein can be used as a marker for PD-L1 expression and optionally for selecting individuals most likely to benefit from anti-αvβ8 treatment.

Anti-αvβ8 antibodies described herein (including αvβ8 binding fragments thereof, affinity matured variants, or scFvs) can be used for diagnosis, either in vivo or in vitro (e.g., using a biological sample obtained from an individual).

When used for detection or diagnosis, the antibody is typically conjugated or otherwise associated with a detectable label. The association can be direct e.g., a covalent bond, or indirect, e.g., using a secondary binding agent, chelator, or linker.

A labeled antibody can be provided to an individual to determine the applicability of an intended therapy. For example, a labeled antibody may be used to detect the integrin 138 density within a diseased area. For therapies intended to target TGF-β or αvβ8 activity (to reduce TGF-β or αvβ8 activity), the density of β8 is typically high relative to non-diseased tissue. A labeled antibody can also indicate that the diseased area is accessible for therapy. Patients can thus be selected for therapy based on imaging results. Anatomical characterization, such as determining the precise boundaries of a cancer, can be accomplished using standard imaging techniques (e.g., CT scanning, MRI, PET scanning, etc.). Such in vivo methods can be carried out using any of the presently disclosed antibodies.

Any of the presently disclosed antibodies can also be used for in vitro diagnostic or monitoring methods, e.g., using cells or tissue from a patient sample. In some embodiments, labeled F9 (or a 138 binding fragment or affinity-matured variant) is used, as it can bind fixed cells as well as non-fixed cells.

In some embodiments, the diagnostic antibody is a single-chain variable fragment (scFv). Intact antibodies (e.g., IgG) can be used for radioimmunotherapy or targeted delivery of therapeutic agents because they exhibit high uptake and retention. In some cases, the persistence in circulation of intact mAbs can result in high background (Olafsen et al. (2012) Tumour Biol. 33:669-77; Cai et al. (2007) J Nucl Med. 48:304-10). ScFvs, typically with a molecular mass of ˜25 kD, are rapidly excreted by the kidneys, but are monovalent and can have lower affinity. The issues of monovalency can be overcome with advanced antibody engineering (as shown herein), where affinities can be improved to the low nM to pM range. Such antibodies have short enough half-lives to be useful as imaging agents and have suitable binding characteristics for tissue targeting (Cortez-Retamozo et al. (2004) Cancer Res. 64:2853-7).

A diagnostic agent including an antibody described herein can include any diagnostic agent known in the art, as provided, for example, in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). The terms “detectable agent,” “detectable moiety,” “label,” “imaging agent,” and like terms are used synonymously herein. A diagnostic agent can be detected by a variety of ways, including as an agent providing and/or enhancing a detectable signal. Detectable signals include, but are not limited to, gamma-emitting, radioactive, echogenic, optical, fluorescent, absorptive, magnetic, or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like. PET is particularly sensitive and quantitative, and thus valuable for characterizing fibrotic processes in vivo (Olafsen et al. (2012) Tumour Biol. 33:669-77; Cai et al. (2007) J Nucl Med. 48:304-10). This is useful beyond a companion diagnostic and would be generally useful to diagnose, clinically stage and follow fibrotic patients during any treatment regimen.

A radioisotope can be incorporated into the diagnostic agents described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F ⁶⁷Ga, ⁶⁸Ga, ³H, ¹⁶⁶Ho, ¹²³I, ¹²⁴I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga. In some embodiments, a nanoparticle can be labeled by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2^{nd} Ed.: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

In some embodiments, a diagnostic agent can include chelators that bind, e.g., to metal ions to be used for a variety of diagnostic imaging techniques. Exemplary chelators include but are not limited to ethylenediaminetetraacetic acid (EDTA), [4-(1,4,8, 11-tetraazacyclotetradec-1-yl) methyl]benzoic acid (CPTA), Cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7, 10-tetraazacyclododecane-1,4,7,10-tetra(methylene phosphonic acid) (DOTP), 1,4,8,11-tetraazacyclotetradecane-1,4,8,11-tetraacetic acid (TETA), 1,4,7,10-tetraazacyclododecane-1,4,7,10-tetraacetic acid (DOTA), N¹,N¹-bis(pyridin-2-ylmethyl)ethane-1,2-diamine (ENPy2) and derivatives thereof.

In some embodiments, the diagnostic agent can be associated with a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Secondary binding ligands include, e.g., biotin and avidin or streptavidin compounds as known in the art.

In some embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present disclosure. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Design of Anti-αvβ8 Antibodies

Anti-αvβ8 antibodies were designed and analyzed. For example, antibody heavy chain variable regions (VH) and light chain variable regions (VL) were designed considering at least the following optimization parameters: (i) concordance with Chothia canonical structures, (ii) the presence of aspartate isomerization motifs, (iii) the presence of asparagine deamidation motifs, (iv) the presence of tryptophan and methionine residues that may be subject to oxidation, (v) the presence of N-linked glycosylation motifs and potential lysine glycation sites, (vi) the presence of unpaired or potentially disulfide-bridged cysteine residues, and (vii) the presence of potentially polyreactive motifs containing at least three aromatic residues. Any sequence liability motifs identified were correlated with structural data in order to provide insight as to how impactful the potential liability might prove, and how to best ameliorate the potential liability.

Humanization was performed using CDR-grafting in order to reduce the immunogenicity of a murine antibody when employed as a human therapeutic. Humanization by CDR-grafting required that the antigen-binding residues from the murine antibody be retained in the humanized antibody; hence, the identification of these antigen-contacting residues was also required. Furthermore, residues in the human acceptor framework (FW) that must be replaced with the cognate murine amino acids in order to permit the murine CDRs to adopt their functional conformation in the context of the human FW were identified.

A cryo-EM structure of the murine antibody in complex with its antigen (6UJB) was used to identify parental murine residues in the human FW that must be conserved, as well as those parental murine residues that may be substituted with their human germline counterparts in the CDRs.

The selection of human acceptor FW regions (FWR) into which the murine CDR regions were grafted was accomplished by searching the IMGT human V gene databases using NCBI IgBLAST employing the reference variable region sequences as input. The strategy was to employ actual human germline sequences, which would not contain the idiosyncratic somatic mutations, found in individual human antibody sequences. Prior CDR-grafting of anti-αvβ8 antibodies appears not to have employed human germline target sequences derived from genome sequencing.

Exemplary antibodies were produced.

Transfection and gene expression was conducted in HEK293 cells and was purified using Protein A and size exclusion chromatography. The resulting proteins were >97% pure with a single major band under non-reducing conditions when analyzed by polyacrylamide gel electrophoresis, and a molecular weight of approximately 150 kD. When evaluated by analytical size exclusion chromatography (SEC), the proteins eluted at a weight of approximately 158 kD. Reducing conditions indicated the presence of the heavy (˜50 kD) and light (˜25 kD) chains in approximately equal ratio.

For testing in mice, murine-human hybrid monoclonal antibodies were produced with the same VH and VL sequences as their humanized counterparts. The variants were also expressed in HEK293 cells and purified by affinity and SEC to a purity >97%. Analysis by gel electrophoresis was consistent with the IgG2a structure (SEC). Gel electrophoresis under reducing conditions indicated the presence of the heavy (˜50 kD) and light (˜25 kD) chains in approximately equal ratio, and a single major band of 150 kD molecular weight under non-reducing conditions.

Antibodies were assessed by surface plasmon resonance (SPR) for the ability to bind to integrin heterodimers. mAbs were immobilized on an anti-Fc CM5 sensor chip, and integrins (0.39-50 nM for and 200 nM for others) were injected at a flow rate of 30 mL/min for a contact time of 180 s and dissociation time of 400 s, at 37° C. Data were fit (grey lines) to a 1:1 antibody:ligand binding model to determine Kd. The results provided in Table 2 show that huAb1, and its murine compatible version, muAb1, have high specificity and low nano-molar binding to αvβ8.

TABLE 2
Surface Plasmon Resonance Binding
Affinities (Kd, nM) of huAb1 and
muAb1 to Human and Murine Integrins

| Antibody | αVβ1 | αVβ3 | αVβ5 | αVβ6 | αVβ8 | mαVβ8 |
|---|---|---|---|---|---|---|
| huAb1 | >200 | >200 | >200 | >200 | <5 | <5 |
| muAb1 | ND | ND | ND | ND | <15 | <15 |

ND = not determined

huAb1 has the light chain variable region (VL) of SEQ ID NO: 1 and the heavy chain variable region (VH) of SEQ ID NO: 2. huAb1 has an IgG4 heavy chain constant domain. The murine-human hybrid monoclonal anti-αVβ8 (muAb1) has the VL sequence of SEQ ID NO: 1 and the VH sequence of SEQ ID NO: 2. muAb1 has an IgG2a heavy chain constant domain.

### Example 2. L-TGFβ Protein Binding Assay

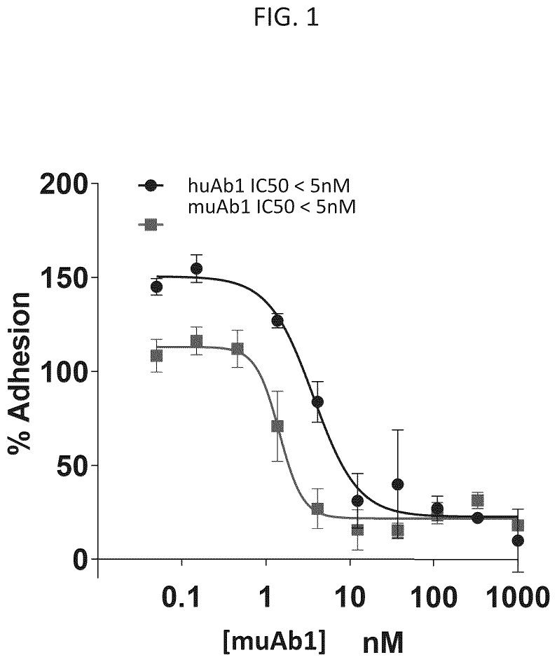

Exemplary antibodies of the disclosure were evaluated for their ability to block binding of the latent form of TGFβ (L-TGFβ) to in a cell-based competition assay. TGFβ is ubiquitously expressed in the latent form and L-TGFβ promotes an immune suppressive phenotype within the tumor microenvironment. L-TGFβ was immobilized on a polystyrene plate and incubated with LN229 cells expressing and increasing concentrations of muAb1 for 30 mins at 37° C. Bound LN229 cells were quantified by Crystal Violet. Data were fit to a 4-parameter displacement curve to determine IC₅₀. The results provided in FIG. 1 show that the LN229 cells which express avb8 on their surface bind to L-TGFb on the plate, and that this interaction is specifically blocked by the anti avb8 integrin antibodies huAb1 and muAb1. The affinities of the two antibodies are similar as measured in this assay.

### Example 3. In Vivo Efficacy and Antitumor Immune Activity in a Pre-Clinical Colorectal Cancer Syngeneic Model

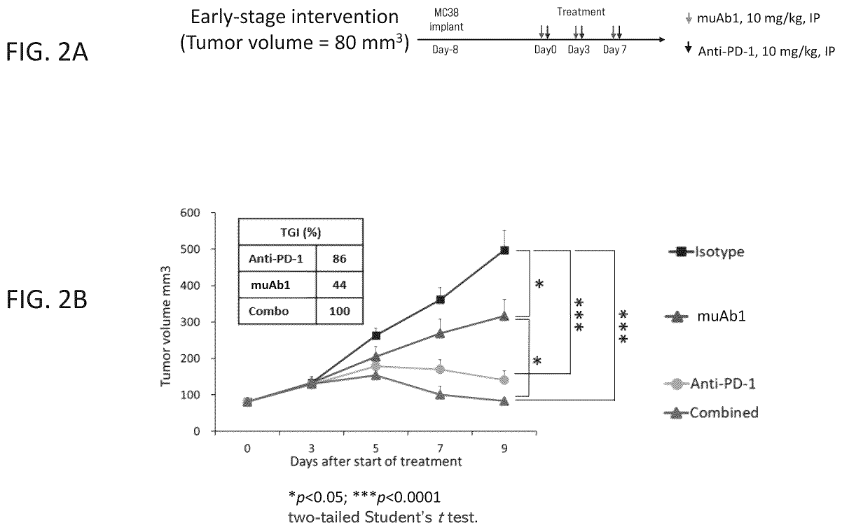

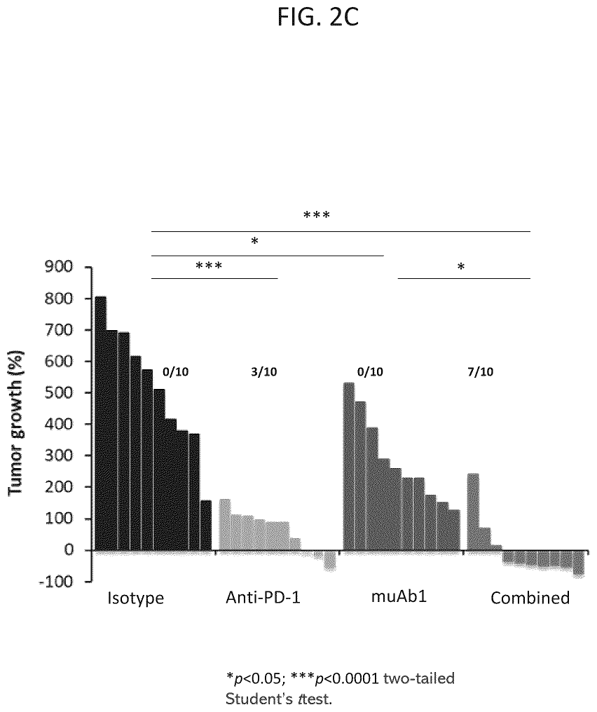

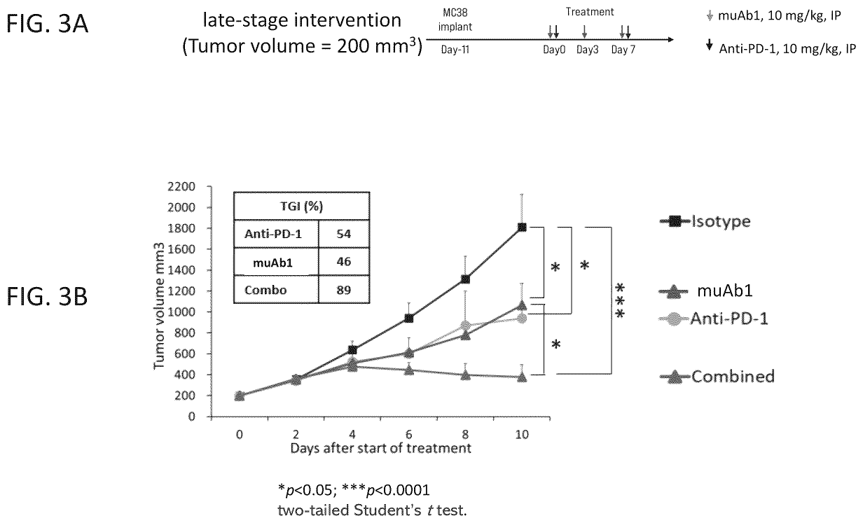

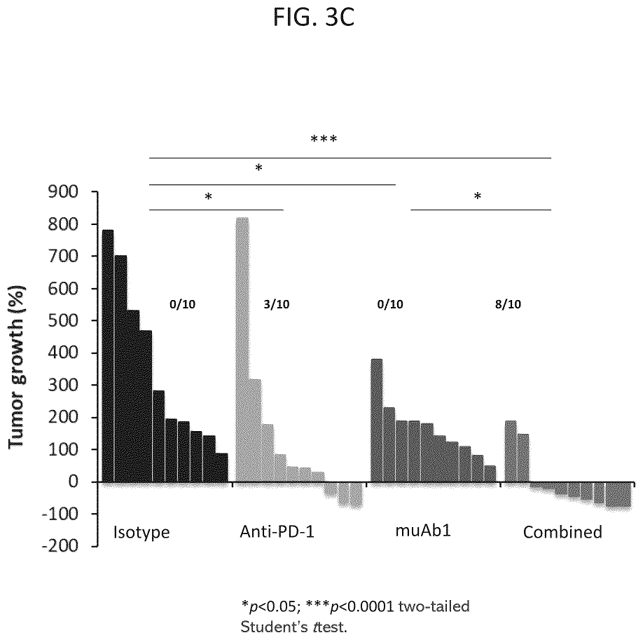

A murine-human hybrid monoclonal anti-αVβ8 antibody (muAb1), which is a murine compatible version of huAb1, was evaluated in a subcutaneous MC38 colorectal cancer syngeneic model in C57BL/6J mice. The objective of the study was to evaluate the anti-tumor efficacy of muAb1 alone or combined with an anti-mPD-1 (RMP1-14) antibody. MC38 tumors were grown to a smaller size (80 mm³, early-intervention protocol, see FIG. 2A) and a larger size (200 mm³, late-intervention protocol, see FIG. 3A) to test efficacy at both early and late stage. This example provides the general experimental protocols for this study. Examples 4-8 provide the results of this study.

Test Article: muAb1 was provided as a solution in PBS pH 7.4. The anti mPD-1 antibody RMP1-14 was provided as a solution in PBS pH 7.0 and rat IgG2a isotype was provided in PBS pH 6.8. The vehicle was PBS pH 7.0.

Cell Culture: The MC38 mouse colorectal tumor cell line (ATCC-MC38, China) was maintained in vitro as a monolayer culture in DMEM medium supplemented with 10% heat inactivated fetal bovine serum, 1% Antibiotic-Antimycotic, and 2 mM L-glutamine at 37° C. in an atmosphere of 5% CO₂ in air. These tumor cells were sub-cultured twice weekly by trypsin-EDTA treatment. The cells growing in an exponential growth phase were harvested and counted for tumor inoculation.

Animals and Tumor Inoculation: For the MC38 tumor model efficacy study female C57BL/6J mice, 6-8 weeks in age and weighing approximately 18-22 g, were inoculated subcutaneously at the right flank with 3×10⁵ MC38 tumor cells in 0.1 mL of PBS. The animals were randomized (n=10/group) and treatment was started when the average tumor volume reached approximately 80 mm³ (FIG. 2A, early-state intervention protocol) or 200 mm³ (FIG. 3A, late-stage intervention protocol).

Endpoints: The major endpoint was to determine if the tumor growth could be delayed, or if mice could be cured. Survival was a secondary endpoint of this study. Tumor sizes were measured thrice weekly in two dimensions using a caliper, and the volume will be expressed in mm³ using the formula: V=0.5 a×b² where a and b are the long and short diameters of the tumor, respectively. The tumor sizes are then used for the calculations of both T/C and TGI values. The T/C value (in percent) is an indication of antitumor effectiveness, T and C are the mean volume of the treated and control groups, respectively, on a given day. TGI was calculated for each group using the formula: TGI (%)=[1−(Ti−T0)/(Vi−V0)]×100; Ti is the average tumor volume of a treatment group on a given day, T0 is the average tumor volume of the treatment group on the first day of treatment, Vi is the average tumor volume of the vehicle control group on the same day with Ti, and V0 is the average tumor volume of the vehicle group on the first day of treatment.

Tumor rechallenge study: Long-term survived tumor-free mice after muAb1 and anti-mPD-1 combination treatment (n=5) were rechallenged by subcutaneously injection with 3×10⁵ MC38 cells on day 52 after the original tumor implantation. Naïve C57BL/6 mice (n=5) subcutaneously injected with 3×10⁵ MC38 cells were served as control.

Statistical Analysis: For comparison between two groups, an independent sample t-test was be used. For comparison among three or more groups, a one-way ANOVA will be performed. If a significant F-statistics (a ratio of treatment variance to the error variance) was obtained, multiple comparison procedures were applied after ANOVA. All data was analyzed using SPSS 17.0. p<0.05 is considered to be statistically significant.

### Example 4. αvβ8-Blockade Inhibits and Augments the Efficacy of PD-1 Immunotherapy in the Pre-Clinical Colorectal Cancer Model (Early-Stage Intervention Study)

The following study was performed as described in Example 3. C57BL/6 mice were inoculated subcutaneously with 0.3×10⁶ MC38 murine colon carcinoma cells. When tumors reached 82±6 mm³ (mean±sem), mice were randomized and treated by intraperitoneal injection (n=10 per treatment) with 10 mg/kg Isotype control, anti-mouse PD-1 mAb (RMP1-14), muAb1 or a combination of anti-mouse PD-1 mAb and muAb1 on days 0, 3 and 7 (FIG. 2A). The results show that αVβ38-blockade with muAb1 inhibits tumor growth and augments the efficacy of PD-1 immunotherapy. Tumor growth curves and tumor growth inhibition (TGI,%) are provided in FIG. 2B. Change in tumor volume compared to baseline in individual mice is provided in FIG. 2C. p values are calculated by unpaired two tail t test. Values are reported as mean±sem. *p<0.05, ***p<0.0001.

### Example 5. αvβ8-Blockade Inhibits and Augments the Efficacy of PD-1 Immunotherapy in the Pre-Clinical Colon Cancer Model (Late-Stage Intervention Study)

The following study was performed as described in Example 3. C57BL/6 mice were inoculated subcutaneously with 0.3×10⁶ MC38 murine colon carcinoma cells. When tumors reached 200±13 mm³ (mean±sem), mice were randomized and treated by intraperitoneal injection (n=10 per treatment) with 10 mg/kg Isotype control, anti-mouse PD-1 mAb (RMP1-14) on days 0 and 7, and 10 mg/kg muAb1 was dosed by intraperitoneal injection on days 0, 3 and 7 (FIG. 3A). The results show that αVβ8-blockade with muAb1 inhibits tumor growth and augments the efficacy of PD-1 immunotherapy. Tumor growth curves and tumor growth inhibition (TGI,%) are provided in FIG. 3B. Change in tumor volume compared to baseline in individual mice is provided in FIG. 3C. p values are calculated by unpaired two tail t test. Values are reported as mean±sem. *p<0.05, ***p<0.0001.

### Example 6. Tumor Reduction Induced by an Anti-αvβ8 Antibody and its Combination with an Anti-PD-1 Antibody is Related to CD8+ T Cell Infiltration and Proliferation within the Tumor Microenvironment

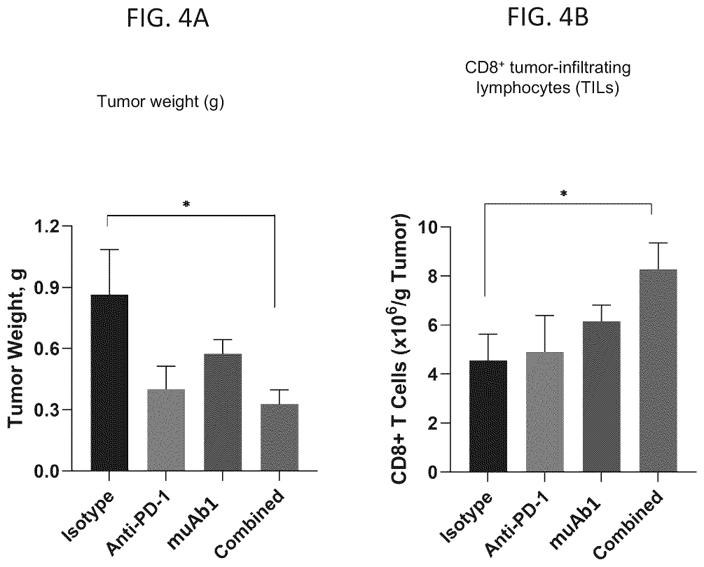

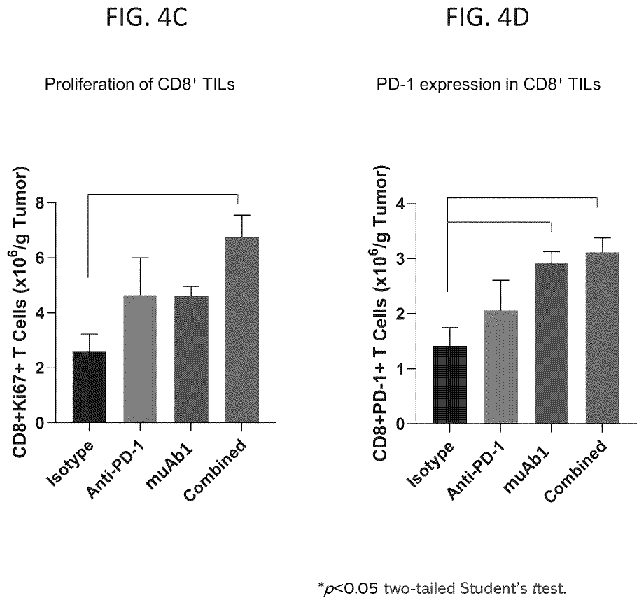

The following study was performed as described in Example 3. C57BL/6 mice were inoculated subcutaneously with 0.3×10⁶ MC38 murine colon carcinoma cells. When tumors reached 280±37 mm³ (mean±sem), mice were randomized and treated by intraperitoneal injection (n=5 per treatment) with 10 mg/kg Isotype control, anti-mouse PD-1 mAb (RMP1-14), muAb1 or combination of anti-mouse PD-1 mAb and muAb1 on days 0 and 3. Tumor-infiltrating lymphoid cells harvested at day 7 were analyzed multicore staining and analysis. Tumor size shrinkage (FIG. 4A) is correlated to CD8+ T-cell infiltration (FIG. 4B) and increased proliferation (FIG. 4C) and activation (FIG. 4D) of these CD8+ T cells within the tumor microenvironment (TME). This example shows that the combination stimulates an influx of CD8+ tumor infiltrating lymphocytes and associated activation within the TME. P values are calculated by unpaired two tail t test. Values are reported as mean±sem. *p<0.05.

### Example 7. Combination Treatment of an Anti-αvβ8 Antibody and an Anti-PD-1 Antibody Elicits Long-Term Antitumor Immunity

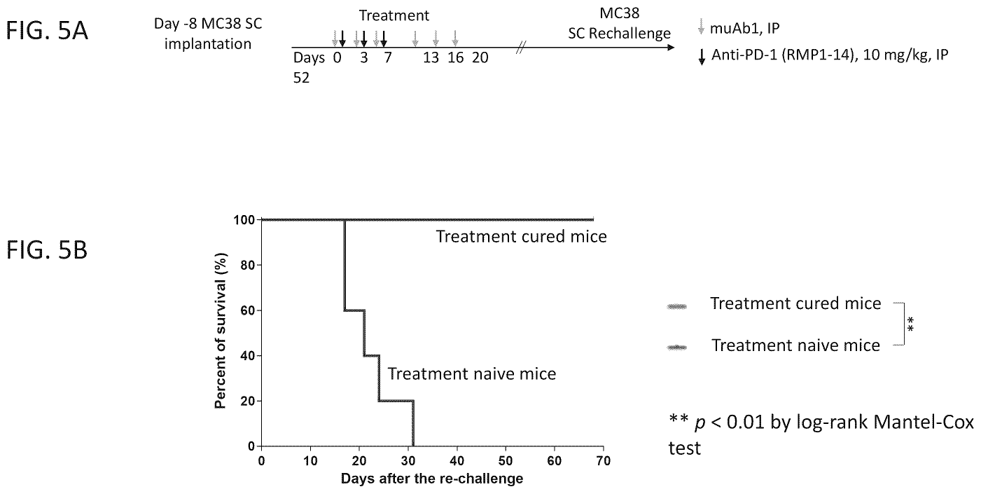

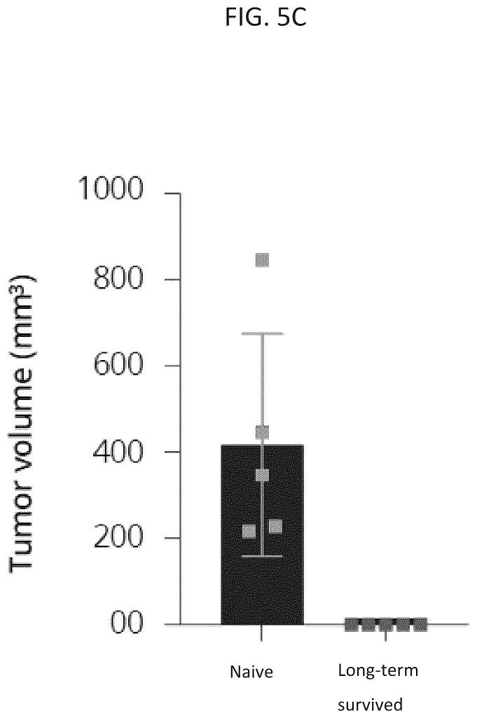

The following study was performed as described in Example 3. Long-term survived tumor-free mice after muAb1 and anti-PD-1 combination treatment (n=5) were re-challenged by subcutaneous injection with 0.3×10⁶ MC38 cells 52 days after original tumor implantation (FIG. 5A). Five naïve C57BL/6 mice subcutaneously inoculated with the same numbers of MC38 cells served as tumor growth control. The results of FIG. 5B show that combination treatment of an anti-αvβ8 antibody and an anti-PD-1 antibody elicits long-term antitumor immunity. The results of FIG. 5C show that tumors did not grow in mice previously treated with the muAb1 and anti-PD-1 mAb combination therapy. P values are calculated by log-rank survival analysis. *p<0.05.

### Example 8. Biodistribution of muAb1 in Mice Bearing MC38

The following study was performed as described in Example 3. C57BL female mice, 6-8 weeks, weighing approximately 18-22 g were subcutaneously inoculated with 3×10⁵ MC38 cells and randomized (n=4/group) and treatment were started when the average tumor volume reaches 450±44 mm³. A single dose of muAb1 or an IgG2 isotype control (10 mg/kg, IP) were administered to MC38 tumor model mice. Blood samples were obtained 24 hr post dose (tumor n=4, plasma n=8) or 72 hr post dose (tumor n=4, plasma n=4).

To quantify the levels of muAb1, mouse integrin αVβ8 heterodimer protein was coated onto an ELISA microplate. Tumor lysate and plasma were pipetted into the wells and analyte present in the samples will bind to the coating ligand. After washing away unbound substances, Peroxidase AffiniPure Goat Anti-Mouse IgG, Fcγ subclass 2a specific was added into the wells to bind with analyte. Following a wash to remove unbound Peroxidase AffiniPure Goat Anti-Mouse IgG, Fcγ subclass 2a specific, a substrate solution was added to the wells and color develops in proportion to the amount of Peroxidase AffiniPure Goat Anti-Mouse IgG, Fcγ subclass 2a specific bound in the initial step. The color development is stopped by addition of sulfuric acid solution and the intensity of the color is measured.

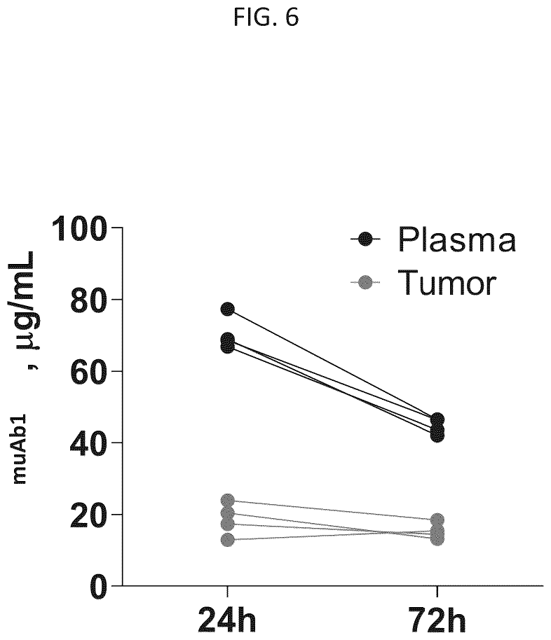

This example shows the biodistribution of muAb1 in plasma and tumor at 24 and 72 hr post intraperitoneal (IP) injection of 10 mg/kg muAb1. The results in FIG. 6 show that the muAb1 effectively distributes to the tumor from the plasma compartment (tumor:plasma ratio is ˜0.25), and this significant muAb1 tumor uptake correlates to antitumor activity. At this particular dose of muAb1, the concentration in the tumor was greater than the antibody binding to integrin αVβ8, thereby accounting for the strong anti-tumor activity.

### Example 9. In Vivo Efficacy in an Immune Excluded Pre-Clinical Breast Cancer Syngeneic Model

A murine-human hybrid monoclonal anti-αVβ8 antibody (muAb1), which is a murine compatible version of huAb1, was evaluated in an orthotopically implanted EMT-6 mouse breast cancer syngeneic model in BALB/c mice. EMT-6 is a known immune excluded tumor model. The objective of the study was to evaluate the anti-tumor efficacy of muAb1 alone or combined with an anti-mPD-1 (RMP1-14) antibody. This example provides the general experimental protocols for this study. Examples 10-12 provide the results of this study.

Test Article: muAb1 was provided as a solution in PBS pH 7.4. The anti mPD-1 antibody RMP1-14 was provided as a solution in PBS pH 7.0 and rat IgG2a isotype was provided in PBS pH 6.8. The vehicle was PBS pH 7.0.

Cell Culture: The EMT-6 mouse breast cancer tumor cell line (ATCC, CRL-2755) was maintained in vitro as a monolayer culture in Waymouth's MB 752/1 medium supplemented with 10% heat inactivated fetal bovine serum, 1% Antibiotic-Antimycotic, and 2 mM L-glutamine at 37° C. in an atmosphere of 5% CO2 in air. The tumor cells were routinely sub-cultured twice weekly by trypsin-EDTA treatment. The cells growing in an exponential growth phase were harvested and counted for tumor inoculation.

Animals and Tumor Inoculation: BALB/c, female, 6-8 weeks, weighing approximately 18-22 g were inoculated in the mammary fat pad were randomized and treatment was started when the average tumor volume reached 60±4 mm³ (n=5/group) or 230±25 mm³ (n=8/group).

Endpoints: The major endpoint was to determine if the tumor growth could be delayed, or if mice could be cured. Survival was a secondary endpoint of this study. Tumor sizes were measured thrice weekly in two dimensions using a caliper, and the volume will be expressed in mm³ using the formula: V=0.5 a×b² where a and b are the long and short diameters of the tumor, respectively. The tumor sizes are then used for the calculations of both T/C and TGI values. The T/C value (in percent) is an indication of antitumor effectiveness, T and C are the mean volume of the treated and control groups, respectively, on a given day. TGI was calculated for each group using the formula: TGI (%)=[1−(Ti−T0)/(Vi−V0)]×100; Ti is the average tumor volume of a treatment group on a given day, T0 is the average tumor volume of the treatment group on the first day of treatment, Vi is the average tumor volume of the vehicle control group on the same day with Ti, and V0 is the average tumor volume of the vehicle group on the first day of treatment.

Statistical Analysis: For comparison between two groups, an independent sample t-test was be used. For comparison among three or more groups, a one-way ANOVA will be performed. If a significant F-statistics (a ratio of treatment variance to the error variance) was obtained, multiple comparison procedures were applied after ANOVA. All data was analyzed using SPSS 17.0. p<0.05 is considered to be statistically significant.

### Example 10. An Anti-αvβ8 Antibody Shows Antitumor Activity as a Single Agent and Reverses Resistance to PD-1 Therapy in an Immune Excluded Breast Tumor Model (Early-Stage Intervention Study)

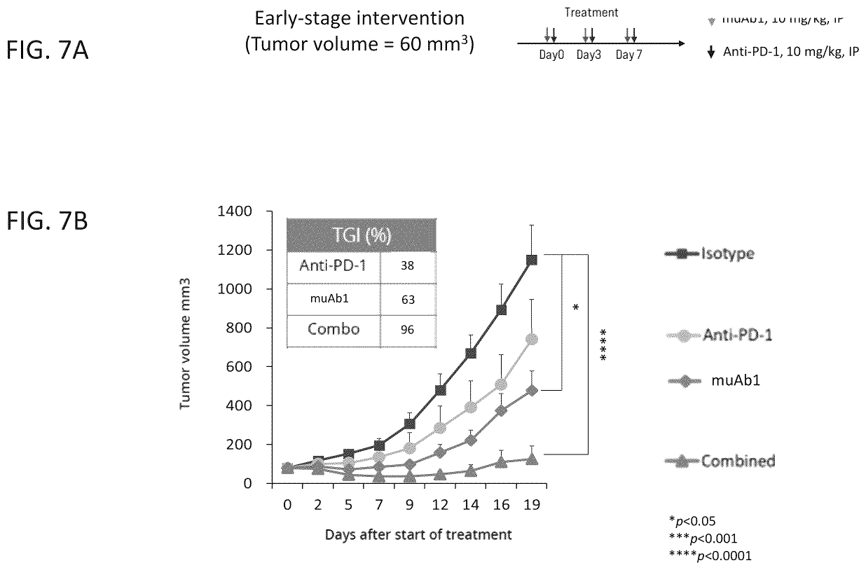

The following study was performed as described in Example 9. Female BALB/c mice were orthotopically inoculated with 0.5×10⁶ EMT6 murine mammary carcinoma cells into the fourth mammary fad pad. When tumors reached 60±4 mm³ (mean±sem), mice were randomized and treated by intraperitoneal injection (n=5 per treatment) with 10 mg/kg Isotype control, anti-mouse PD-1 mAb (RMP1-14), muAb1 or a combination of anti-mouse PD-1 mAb and muAb1 on days 0, 3 and 7 (FIG. 7A). p values are calculated by unpaired two tail t test. The results of this study demonstrate that the muAb1 antibody shows antitumor activity as a single agent and reverses resistance to PD-1 therapy in an immune excluded breast tumor model (FIG. 7B). Additionally, the combination with muAb1 affords higher anti-tumor activity than muAb1 alone. Values are reported as mean±sem. *p<0.05, ***p<0.001, ****p, 0.0001.

### Example 11. An Anti-αvβ8 Antibody Shows Antitumor Activity as a Single Agent and Reverses Resistance to PD-1 Therapy in an Immune Excluded Breast Tumor Model (Late-Stage Intervention Study)

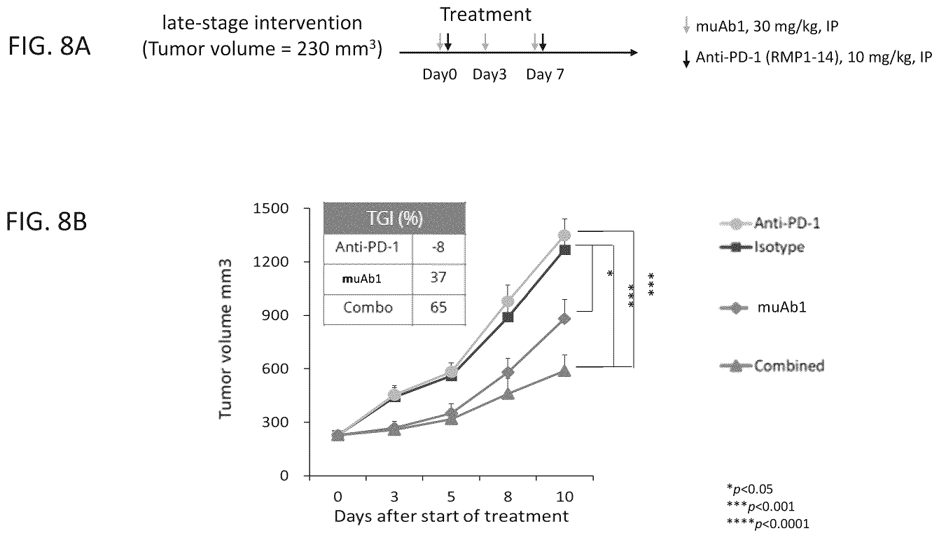

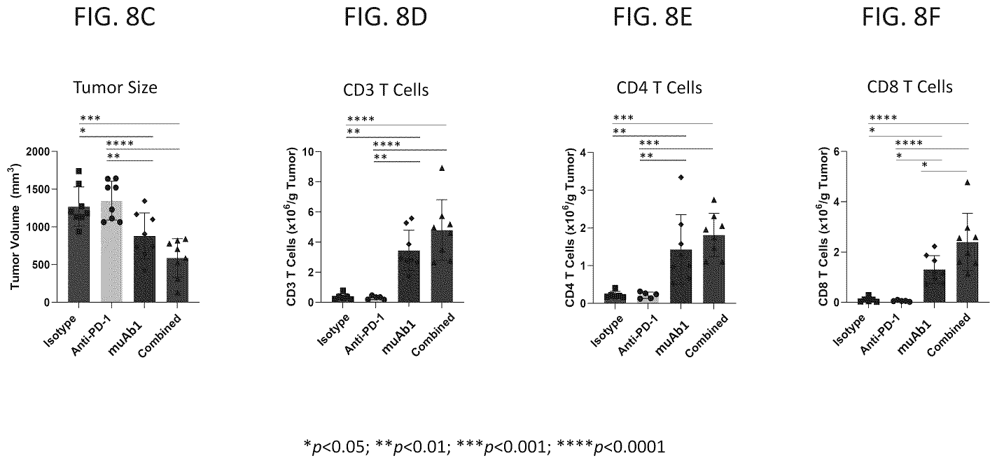

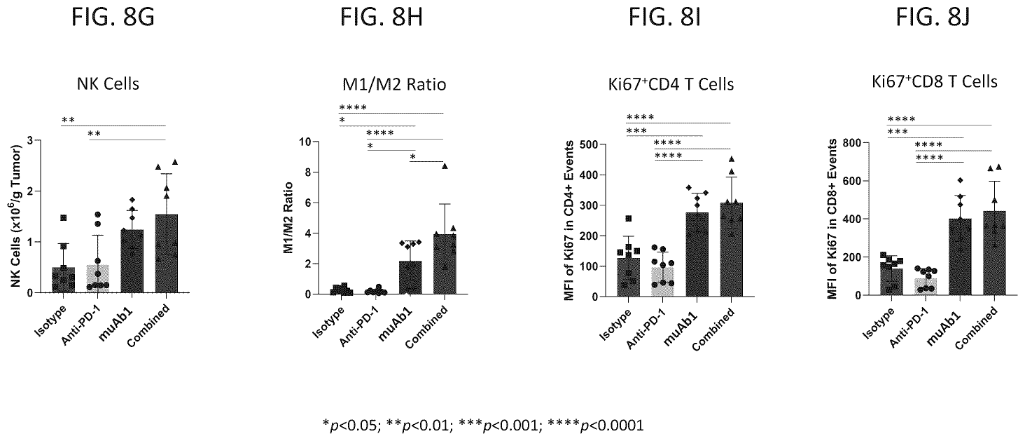

The following study was performed as described in Example 9. Female BALB/c mice were orthotopically inoculated with 0.5×10⁶ EMT6 murine mammary carcinoma cells into the fourth mammary fad pad. When tumors reached 230±25 mm³ (mean±sem), mice were randomized and treated by intraperitoneal injection (n=8 per treatment) with 10 mg/kg Isotype control, anti-mouse PD-1 mAb (RMP1-14) on days 0 and 7, and 30 mg/kg muAb1 was dosed by intraperitoneal injection on days 0, 3 and 7 (FIG. 8A). p values are calculated by unpaired two tail t test. The results of this study demonstrate that the muAb1 antibody shows antitumor activity as a single agent and reverses resistance to PD-1 therapy in an immune excluded breast tumor model (FIG. 8B). Whereas PD-1 therapy alone in this model is ineffective at suppressing tumor growth (TGI is similar to isotype control), the combination with muAb1 affords higher anti-tumor activity than muAb1 alone. Values are reported as mean±sem. *p<0.05, ***p<0.001, ****p<0.0001.

Example 12. An Anti-αvβ8 Antibody, Alone and in Combination with an Anti-PD-1 Antibody, Stimulated Infiltration and Expansion of T Cells, NK Cells and M1 Polarized Macrophages in an Immune Excluded Breast Tumor Model (Late-Stage Intervention Study)

The following study was performed as described in Example 9. BALB/c female mice (n=8/group) bearing orthotopically implanted EMT6 murine breast tumors were treated by intraperitoneal injection with 10 mg/kg Isotype control, 10 mg/kg anti-mouse PD-1 mAb (RMP1-14), 30 mg/kg muAb1 or combination of anti-mouse PD-1 mAb and muAb1 on days 0, 3 and 7. Tumor infiltrating lymphocytes dissociated from tumors collected on day 10 were analyzed by flow cytometry analysis for T cells, NK cells and M1 or M2 polarized macrophages. The results of this study demonstrate that EMT-6 tumors post treatment showed a reshaped tumor microenvironment (TME), including marked increases in infiltrations of T cells, NK cells and M1 polarized macrophages in muAb1 m when administered along or in combination with anti-PD-1 treated tumors (FIGS. 8C-8J). The p values are calculated by one-way ANOVA followed by Tukey's multiple-comparison test. *p<0.05, **p<0.01, ***p<0.001, ****p<0.0001.

### Example 13. In Vivo Efficacy in a Pre-Clinical Triple Negative Breast Cancer Syngeneic Model that is Resistant to Treatment with Immune Checkpoint Inhibitors

A murine-human hybrid monoclonal anti-αVβ8 antibody (muAb1), which is a murine compatible version of huAb1, was evaluated in an orthotopically implanted 4T1 mouse triple-negative breast cancer syngeneic model in BALB/c mice. EMT-6 is a known immune desert tumor model that is resistant to treatment with immune checkpoint inhibitors. The objective of the study was to evaluate the anti-tumor efficacy of muAb1 alone or combined with an anti-mPD-1 (RMP1-14) antibody. This example provides the general experimental protocols for this study. Example 14 provides the results of this study.

Test Article: muAb1 was provided as a solution in PBS pH 7.4. The anti mPD-1 antibody RMP1-14 was provided as a solution in PBS pH 7.0 and rat IgG2a isotype was provided in PBS pH 6.8. The vehicle was PBS pH 7.0.

Cell Culture: The 4T1 mouse breast cancer tumor cell line (ATCC, CRL-2539). 4T1 was maintained in vitro as a monolayer culture in RPMI-1640 medium supplemented with 10% heat inactivated fetal bovine serum, 1% Antibiotic-Antimycotic, and 2 mM L-glutamine at 37° C. in an atmosphere of 5% CO2 in air. The tumor cells were routinely sub-cultured twice weekly by trypsin-EDTA treatment. The cells growing in an exponential growth phase were harvested and counted for tumor inoculation.

Animals and Tumor Inoculation: BALB/c, female, 6-8 weeks, weighing approximately 18-22 g were inoculated in the mammary fat pad and randomized (n=10/group) to treatment groups. Drug treatments were started when the average tumor volume reaches 50±2 mm³. The test article administration and the animal numbers in each group are shown in the following experimental design table.

Endpoints: The major endpoint was to determine if the tumor growth could be delayed, or if mice could be cured. Survival was a secondary endpoint of this study. Tumor sizes were measured thrice weekly in two dimensions using a caliper, and the volume will be expressed in mm³ using the formula: V=0.5 a×b² where a and b are the long and short diameters of the tumor, respectively. The tumor sizes are then used for the calculations of both T/C and TGI values. The T/C value (in percent) is an indication of antitumor effectiveness, T and C are the mean volume of the treated and control groups, respectively, on a given day. TGI was calculated for each group using the formula: TGI (%)=[1−(Ti−T0)/(Vi−V0)]×100; Ti is the average tumor volume of a treatment group on a given day, TO is the average tumor volume of the treatment group on the first day of treatment, Vi is the average tumor volume of the vehicle control group on the same day with Ti, and V0 is the average tumor volume of the vehicle group on the first day of treatment.

Statistical Analysis: For comparison between two groups, an independent sample t-test will be used. For comparison among three or more groups, a one-way ANOVA will be performed. p<0.05 is considered to be statistically significant.

Example 14. An Anti-αvβ8 Antibody Shows Antitumor Activity as a Single Agent and Reverses Resistance to PD-1 Therapy in an Immune Desert Triple Negative Breast Tumor Model (Early-Stage Intervention Study)

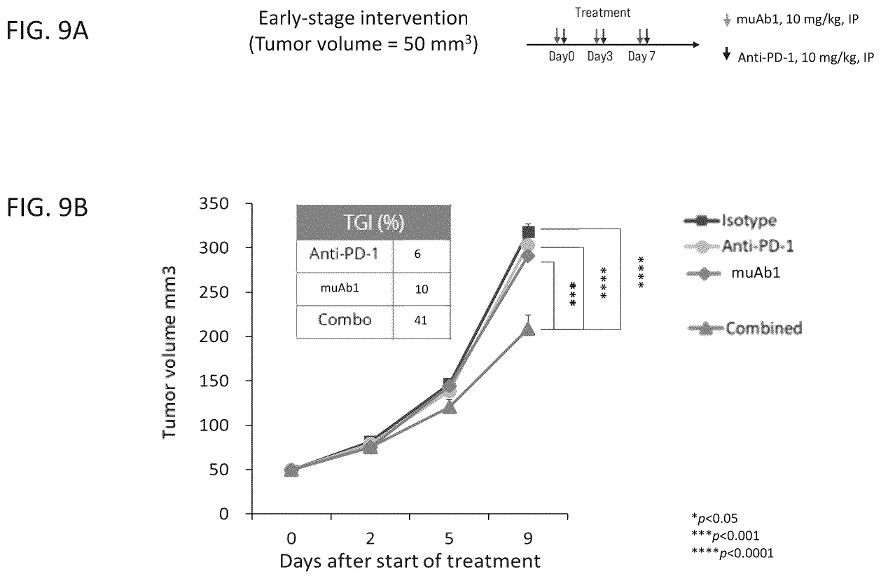

The following study was performed as described in Example 13. Female BALB/c mice were orthotopically inoculated with 4T1 murine mammary carcinoma cells into the mammary fad pad. When tumors reached 50±2 mm³ (mean±sem), mice were randomized and treated by intraperitoneal injection (n=10 per treatment) with 10 mg/kg Isotype control, anti-mouse PD-1 mAb (RMP1-14), muAb1 or a combination of anti-mouse PD-1 mAb and muAb1 on days 0, 3 and 7 (FIG. 9A). p values are calculated by unpaired two tail t test. The results of this study demonstrate that the combination of muAb1 antibody with anti-PD-1 therapy resulted in significant delay in tumor growth in an immune desert tumor model that is non-responsive to current checkpoint blockade therapies (FIG. 91B). Values are reported as mean±sem. *p<0.05, ****p<0.001, ****p, 0.0001.

### Example 15. Pharmaceutical Compositions of Anti-αvβ8 Antibodies

Pharmaceutical compositions of huAb1 were formulated and tested to determine particle formation from huAb1 aggregation and overall huAb1 stability. The composition of the test formulations are given in Table 3.

The formulations included a buffer (phosphate buffer or histidine buffer), a nonionic surfactant (polysorbate surfactants, polyglycolized glycerides, alkyl saccharides, or ester saccharides), and a nonionic tonicity agent (sucrose or trehalose). Arginine, glutamate, or NaCl were optionally included. The compositions were formulated at a pH of 6.0 or 6.5.

TABLE 3
Tested formulations of huAb1.

| Formulation | Formulation Composition |
|---|---|
| 1 | 20 mM Sodium Phosphate, 5% (w/w) Sucrose, 50 mM NaCl, 0.02% (w/w) |
| | polyoxyethylene 20 sorbitan monolaurate, pH 6.0 |
| 2 | 20 mM Sodium Phosphate, 7.5% (w/w) Sucrose, 0.02% (w/w) polyoxyethylene |
| | 20 sorbitan monolaurate, pH 6.0 |
| 3 | 20 mM Sodium Phosphate, 5% (w/w) Sucrose, 50 mM Arginine, 0.02% (w/w) |
| | polyoxyethylene 20 sorbitan monolaurate, pH 6.0 |
| 4 | 20 mM Sodium Phosphate, 5% (w/w) Sucrose, 50 mM Glutamate, 0.02% (w/w) |
| | polyoxyethylene 20 sorbitan monolaurate, pH 6.0 |
| 5 | 20 mM Sodium Phosphate, 5% (w/w) Sucrose, 50 mM NaCl, 0.02% (w/w) |
| | polyoxyethylene 20 sorbitan monooleate, pH 6.0 |
| 6 | 20 mM Sodium Phosphate, 5% (w/w) Trehalose, 50 mM NaCl, 0.02% (w/w) |
| | polyoxyethylene 20 sorbitan monolaurate, pH 6.0 |
| 7 | 20 mM Sodium Phosphate, 5% (w/w) Sucrose, 50 mM NaCl, 0.02% (w/w) |
| | polyoxyethylene 20 sorbitan monolaurate, pH 6.5 |
| 8 | 20 mM Sodium Phosphate, 5% (w/w) Sucrose, 50 mM NaCl, 0.02% (w/w) |
| | polyoxyethylene 20 sorbitan monooleate, pH 6.5 |
| 9 | 20 mM Sodium Phosphate, 5% (w/w) Sucrose, 50 mM Arginine, 0.02% (w/w) |
| | polyoxyethylene 20 sorbitan monolaurate, pH 6.5 |
| 10 | 20 mM Sodium Phosphate, 5% (w/w) Sucrose, 50 mM Glutamate, 0.02% (w/w) |
| | polyoxyethylene 20 sorbitan monolaurate, pH 6.5 |
| 11 | 20 mM Sodium Phosphate, 5% (w/w) Trehalose, 50 mM NaCl, 0.02% (w/w) |
| | polyoxyethylene 20 sorbitan monolaurate, pH 6.5 |
| 12 | 20 mM Histidine, 5% (w/w) Sucrose, 50 mM Arginine, 0.02% (w/w) |
| | polyoxyethylene 20 sorbitan monolaurate, pH 6.5 |

Differential scanning calorimetry (DSC) assays were run to determine the melting temperature of huAb1 in each formulation. The DLS results indicated that huAb1 was the most stable (highest melting temperature) in formulations containing sodium phosphate at pH 6.5.

Several assays were carried out to determine the extent of huAb1 aggregation in each formulation. Micro-Cuvette based Composition-Gradient Multiangle Light Scattering (CG-MALS) assays were performed to determine the apparent molecular weight of huAb1 in the formulation. The angular dependence of the light scattering signal is converted to apparent molecular weight of the molecule; the direction of the change in apparent molecular weight versus protein concentration provides evidence for attractive or repulsive protein:protein interactions. Results from CG-MALS assays indicated that the presence of arginine in the formulation prevented huAb1 aggregation. Furthermore, formation of sub-visible particulates (SVP) was determined using background-subtracted membrane imaging. The SVP stability results indicated that the presence of sodium chloride accelerated formation of particles, indicating huAb1 aggregation.

Formula 9 yielded superior results over all tested formulations, with low particle formulation and low opalescence, even under stress conditions including high temperature, agitation, and freeze-thaw.

### Example 16. In Vivo Efficacy in a Pre-Clinical Ovarian Cancer Orthotopic Model

A murine-human hybrid monoclonal anti-αVβ8 antibody (muAb1), which is a murine compatible version of huAb1, was evaluated in an ovarian cancer model in C57BL/6 mice. The objective of the study was to evaluate the in vivo anti-tumor efficacy of muAb1 in subcutaneous xID8 tumor-bearing female C57BL/6 mice. This example provides the general experimental protocols for this study. Example 17 provides the results of this study.

Experimental Design: The test article administration route, dose, dosing schedule and animal numbers in each group are shown in Table 4. IP=intraperitoneal injection; BIW=twice a week.

TABLE 4
Description of experimental design

| | n (number | | | | | |
|---|---|---|---|---|---|---|
| | of animals/ | | Dose | Dosing | Dosing | |
| Group | group) | Treatment | (mg/kg) | Volume | Route | Schedule |
| 1 | 10 | rat IgG2a | 10 | 5 μL/g | IP | BIW × 7 doses |
| | | Isotype | | | | |
| 2 | 10 | muAb1 | 10 | 5 μL/g | IP | BIW × 7doses |

Test Article: muAb1 was provided in PBS. Rat IgG2a isotype was provided in PBS.

Cell culture: The xID8 mouse ovarian cancer cell line (derived from ID8 tumor through once in vivo passage), was maintained in vitro as a monolayer cultured in DMEM medium supplemented with 10% heat inactivated fetal bovine serum and 1% Antibiotic-Antimycotic at 37° C. in an atmosphere of 5% CO2 in air. The tumor cells were routinely subcultured twice weekly by trypsin-EDTA treatment. The cells growing in an exponential growth phase were harvested and counted for tumor inoculation.

Animals and Tumor Inoculation: 9 week old, female C57BL/6 mice weighing between 17.3-20.9 g were inoculated subcutaneously at right upper flank with xID8 tumor cells (5×10⁶) in 0.1 mL of PBS for tumor development. Animals were assigned into two separate groups to evaluate anti-tumor efficacy, the animals were randomized and treatment was initiated when the average tumor volume reached approximately 80 mm³.

Endpoints: The major endpoint was to determine the effects of muAb1 treatment on tumor growth. Tumor size was measured twice weekly in two dimensions using a caliper, and the volume was expressed in mm³ using the formula: V=0.5 a×b² where a and b are the long and short diameters of the tumor, respectively. The tumor size was then used for the calculations of both T/C and TGI values. The T/C value (in percent) is an indication of antitumor effectiveness, T and C are the mean volumes of the treated and control groups, respectively, on a given day. TGI was calculated for each group using the formula: TGI (%)=[1−(Ti−T0)/(Vi−V0)]×100; Ti is the average tumor volume of a treatment group on a given day, TO is the average tumor volume of the treatment group on the first day of treatment, Vi is the average tumor volume of the vehicle control group on the same day with Ti, and V0 is the average tumor volume of the vehicle control group on the first day of treatment.

Statistical Analysis: Summary statistics, including mean and the standard error of the mean (SEM), are provided for the tumor volume of each group at each time point. Statistical analysis of difference in the tumor volume among the groups were conducted on the data obtained on day 21 after the start of treatment in xID8 tumor-bearing C57BL/6 mice. One-way ANOVA was performed to compare the tumor volume among groups, and when a significant F-statistic (a ratio of treatment variance to the error variance) was obtained, comparisons between groups were carried out with t-test. All data were analyzed using prism 8.0. p<0.05 was considered to be statistically significant.

### Example 17. An Anti-αvβ8 Antibody Shows Anti-Tumor Activity in a Pre-Clinical Ovarian Cancer Model

The following study was performed as described in Example 16 to evaluate the therapeutic efficacy of muAb1 in xID8 tumor model in C57BL/6 mice. Female C57BL/6 mice were inoculated subcutaneously with 5×10⁶ xID8 tumor cells. When tumors reached 80 mm³, mice were randomized and treated by intraperitoneal injection (n=10 per treatment) with 10 mg/kg isotype control or muAb1 twice weekly for a total of 7 doses.

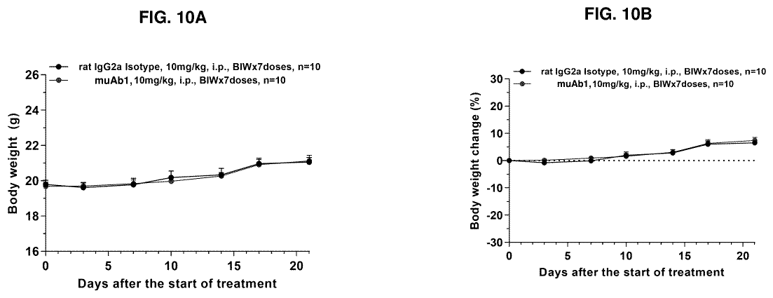

Animal body weight was monitored regularly as an indirect measure of toxicity. No obvious bodyweight loss was observed in all groups. There was no animal death resulted from drug administration. Body weight changes of different treatment groups are shown in FIGS. 10A-10B.

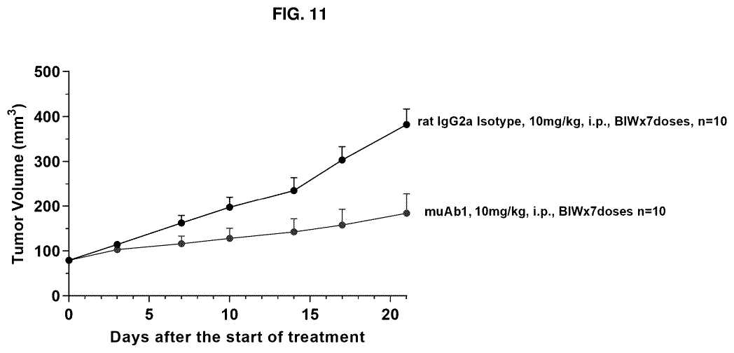

Mean tumor volume over time in female C57BL/6 mice bearing xID8 tumors dosed with muAb1 is shown in Table 5. Tumor growth curves are shown in FIG. 11.

TABLE 5

| Days after start of | Tumor volume (mm³) (mean ± SEM) | | | | | | |
|---|---|---|---|---|---|---|---|
| treatment | 0 | 3 | 7 | 10 | 14 | 17 | 21 |
| rat IgG2a Isotype, | 80 ± 3 | 115 ± 6 | 162 ± 17 | 198 ± 22 | 235 ± 29 | 303 ± 30 | 382 ± 35 |
| 10 mg/kg, IP, | | | | | | | |
| BIW × 7 doses | | | | | | | |
| muAb1, 10 mg/kg, IP, | 79 ± 3 | 103 ± 7 | 116 ± 17 | 128 ± 22 | 142 ± 29 | 158 ± 35 | 184 ± 44 |
| BIW × 7 doses | | | | | | | |

Analysis of tumor growth inhibition is shown in Table 6.

TABLE 6
Tumor growth inhibition calculation for muAb1
mono therapy in xID8 tumor model calculated
based on tumor volume measurements on day 21

| | Tumor Size | T/C^{b} | TGI | p value^{d} |
|---|---|---|---|---|
| Treatment | (mm³)^{a} at day 21 | (%) | %^{c} | T test |
| rat IgG2a Isotype, | 382 ± 35 | — | — | — |
| 10 mg/kg, IP, | | | | |
| BIWx7doses | | | | |
| muAb1, 10 mg/kg, | 184 ± 44 | 48.18 | 65.43 | 0.0023(**) |
| IP, BIWx7doses | | | | |

^{a}Mean ± SEM.
^{b}Tumor Growth Inhibition is calculated by dividing the group average tumor volume for the treated group by the group average tumor volume for the control group (T/C).
^{c}TGI (%) = [1 − (T₂₁ −T₀)/(V₂₁ − V₀)] × 100; T₂₁ is the average tumor volume of a treatment group on day 21, T₀ is the average tumor volume of the treatment group on day 0 after treatment, V₂₁ is the average tumor volume of the vehicle control group on the same day with T₂₁, and V₀ is the average tumor volume of the vehicle control group on day 0 after treatment.
^{d}All data were analyzed using Prism 8.0.1 Comparisons between groups were carried out with t-test when homogeneity of variances was not assumed.
(p = 0.0023(**)).

Tumor weights are summarized in Table 7.

TABLE 7
Tumor weights (g) of different treatment
groups at termination of study (Day 23)

| | Tumor Weight (g)^{a} | T/C^{b} | p value^{c} |
|---|---|---|---|
| Treatment | (at day 23) | (%) | T test |
| rat IgG2a Isotype, | 0.466 ± 0.040 | — | — |
| 10 mg/kg, IP, | | | |
| BIWx7doses | | | |
| muAb1, 10 mg/kg, | 0.185 ± 0.054 | 39.70 | p < 0.0001(****) |
| IP, BIWx7doses | | | |

^{a}Mean ± SEM.
^{b}Tumor Growth Inhibition is calculated by dividing the tumor weight of the treated group by the tumor weight of vehicle control group (T/C_{weight}).
^{c}All data were analyzed using Prism 8.0.1. Comparisons between groups were carried out with T test when homogeneity of variances was not assumed.
(p < 0.0001(****)).

In summary, there was no mortality, and muAb1 was tolerated well by the tumor-bearing animals. No body weight loss was observed in enrolled animals. The results of tumor sizes in different groups post the start of treatment are shown in Tables 5-7 and FIG. 11.

The mean tumor size of vehicle control group (rat IgG2a Isotype) reached 382 mm³ on day 21 of treatment. Monotherapy of muAb1 at 10 mg/kg produced significant anti-tumor activity. The mean tumor size was 184 mm³ on day 21 (T/C value=48.18%; TGI=65.43%; p=0.0023, compared with the control group).

### Example 18. In Vivo Efficacy and Pharmacodynamics in a Pre-Clinical Glioblastoma Cancer Syngeneic Model

A murine-human hybrid monoclonal anti-αVβ8 antibody (muAb1), which is a murine compatible version of huAb1, was evaluated in an subcutaneous GL261 glioblastoma model in C57BL/6 mice. The objective of the study was to evaluate the in vivo anti-tumor efficacy of muAb1 alone or combined with an anti-mPD-1 antibody in subcutaneous xID8 tumor-bearing female C57BL/6 mice. In addition, the in vivo pharmacodynamics of muAb1 were characterized in tumor and serum samples. This example provides the general experimental protocols for this study. Example 19 provides the results of the efficacy study, and Example 20 provides the results of the PD study.

Experimental Design: The test article administration route, dose, dosing schedule and animal numbers in each group are shown in Tables 8 and 9. QW=once a week; BIW=twice a week; IP=intraperitoneal injection.

TABLE 8
Description of experimental design (efficacy study- Example 19)

| | n (number of | | Dose | Dosing | Dosing | |
|---|---|---|---|---|---|---|
| Group | animals/group) | Treatment | (mg/kg) | Volume | Route | Schedule |
| 1 | 10 | Vehicle + Rat IgG2a | 0 + 10 | 5 μL/g | IP | QW × 3W + |
| | | Isotype | | | | BIW × 3W |
| 2 | 10 | Anti-mPD-1 + Rat | 10 + 10 | 5 μL/g | IP | QW × 3W + |
| | | IgG2a Isotype | | | | BIW × 3W |
| 3 | 10 | muAb1 + Rat IgG2a | 10 + 10 | 5 μL/g | IP | BIW × 3W |
| | | Isotype | | | | |
| 4 | 10 | Anti-mPD-1 + muAb1 | 10 + 10 | 5 μL/g | IP | QW × 3W + |
| | | | | | | BIW × 3W |

TABLE 9
Description of experimental design (PD study- Example 20)

| | n (number | | | | | |
|---|---|---|---|---|---|---|
| | of animals/ | | Dose | Dosing | Dosing | |
| Group | group) | Treatment | (mg/kg) | Volume | Route | Schedule |
| 1 | 5 | Rat IgG2a | 10 | 5 μL/g | IP | BIW × 2W |
| | | Isotype | | | | |
| 2 | 5 | muAb1 | 10 | 5 μL/g | IP | BIW × 2W |

Test Article: muAb1 was provided in PBS pH 7.4. Anti-PD-1 was provided in PBS. Rat IgG2a isotype was provided in PBS.

Cell culture: The GL261 mouse glioma cells (CoBioer, CBP60669) were maintained in vitro as a monolayer culture in DMEM medium supplemented with 10% heat inactivated fetal bovine serum, 1% Antibiotic-Antimycotic, and 4 μM L-glutamine at 37° C. in an atmosphere of 5% CO₂ in air. The tumor cells were routinely subcultured twice weekly by trypsin-EDTA treatment. The cells growing in an exponential growth phase were harvested and counted for tumor inoculation.

Animals and Tumor Inoculation: 9 week old, female C57BL/6 mice weighing between 17.0-20.9 g were inoculated subcutaneously at right flank with GL261 tumor cells (0.5×10⁶) in 0.1 mL of PBS for tumor development. Treatments were started on day 7 after tumor inoculation when the average tumor size reached 81 mm³ in the efficacy study. Treatments were started on day 9 after tumor inoculation when the average tumor size reached 79 mm³ in the PD study. The animals were assigned into groups using an Excel-based randomization software performing stratified randomization based upon their tumor volumes. The testing article was administrated to the mice according to the predetermined regimen as shown in the experimental design table (Tables 8 and 9).

Tumor Measurements and Endpoints: The major endpoint was to determine the effects of muAb1 treatment on tumor growth. Tumor size was measured thrice weekly in two dimensions using a caliper, and the volume was expressed in mm³ using the formula: V=0.5 a×b² where a and b are the long and short diameters of the tumor, respectively. The tumor size was then used for the calculations of both T/C and TGI values. The T/C value (in percent) is an indication of antitumor effectiveness, T and C are the mean volumes of the treated and control groups, respectively, on a given day. TGI was calculated for each group using the formula: TGI (%)=[1−(Ti−T0)/(Vi−V0)]×100; Ti is the average tumor volume of a treatment group on a given day, TO is the average tumor volume of the treatment group on the first day of treatment, Vi is the average tumor volume of the vehicle control group on the same day with Ti, and V0 is the average tumor volume of the vehicle control group on the first day of treatment.

Statistical Analysis: Summary statistics, including mean and the standard error of the mean (SEM), are provided for the tumor volume of each group at each time point. Statistical analysis of difference in the tumor volume among the groups were conducted on the data obtained on day 14 after the start of treatment in efficacy study. Statistical analysis of difference in the tumor volume among the groups were conducted on the data obtained on day 13 after the start of treatment in PD study. A one-way ANOVA was performed to compare the tumor volume among groups (23), and when a significant F-statistic (a ratio of treatment variance to the error variance) was obtained, comparisons between groups were carried out with Games-Howell test. Independent-samples T test was performed to compare the tumor volume between groups (=2). p<0.05 was considered to be statistically significant.

PD Sampling and Analysis: For tumor sample collection (QPCR), tumor samples were taken from animals in Group 1 (n=5/group) and Group 2 (n=4/group) on day 13, stored in Trizol at −80° C. for future RNA isolation and real-time quantitative PCR examination. For tumor sample collection (PD assay), tumor samples were taken from animals in Group 1 (n=5/group) and Group 2 (n=4/group) on day 13 for TGF-β1 and VEGF ELISA assay and immunoblotting analysis of p/tSmad2, p/tSmad3 and Sox4. For serum preparation (PD assay), terminal fresh whole blood sample was taken from animals in Group 1 (n=5/group) and Group 2 (n=4/group) on day 13. Blood samples were left on ice for 30 min before centrifugation at 3000 rpm, 4° C. for 10 min. Then, serum samples were transferred into new tubes and kept frozen at −80° C. before TGF-β1 and VEGF ELISA assay.

### Example 19. Combination Treatment of an Anti-αvβ8 Antibody and an Anti-PD-1 Antibody Shows Anti-Tumor Activity and Improves Survival in the Pre-Clinical Glioblastoma Cancer Model

The following study was performed as described in Example 18 to evaluate the therapeutic efficacy of muAb1 alone or combined with an anti-mPD-1 antibody in GL261 glioblastoma model in C57BL/6 mice. Female C57BL/6 mice were inoculated subcutaneously with 0.5×10⁶ GL261 tumor cells. When tumors reached 81 mm³, mice were randomized and treated by intraperitoneal injection (n=10 per treatment) with one of the following dosing groups: (1) vehicle (once weekly for 3 weeks) and 10 mg/kg isotype control (twice weekly for 3 weeks); (2) 10 mg/kg anti-mouse PD-1 mAb (once weekly for 3 weeks) and 10 mg/kg isotype control (twice weekly for 3 weeks); (3) 10 mg/kg muAb1 (twice weekly for 3 weeks) and 10 mg/kg isotype control (twice weekly for 3 weeks); or (4) 10 mg/kg anti-mouse PD-1 mAb (once weekly for 3 weeks) and 10 mg/kg muAb1 (twice weekly for 3 weeks).

### Mortality, Morbidity, and Body Weight Gain or Loss

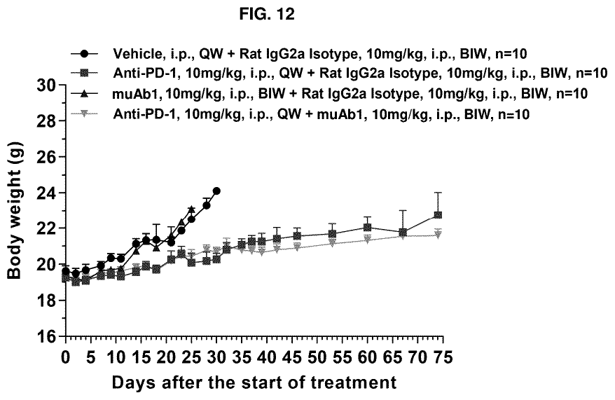

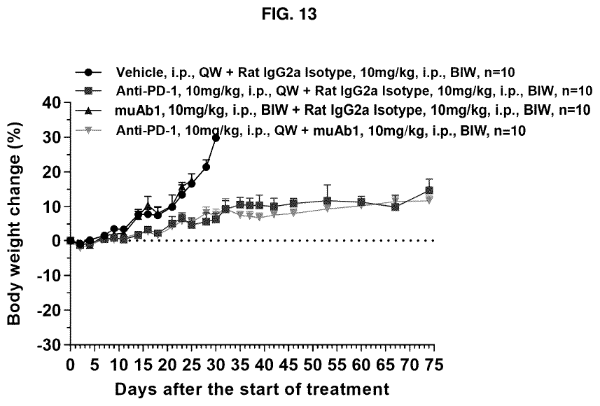

Animal body weight was monitored regularly as an indirect measure of toxicity. In the efficacy study, one animal in dosing group 4 was found dead on day 17. Study was ended on day 74 by euthanizing all the remaining animals. None of the animals exhibited abnormal behavior during the study. Body weight changes in female C57BL/6 mice bearing GL261 established tumors dosed according to the treatment groups is shown in FIGS. 12 and 13.

### Tumor Volume

Mean tumor volume over time in female C57BL/6 mice bearing GL261 established tumors dosed according to the treatment groups is shown in Table 10.

TABLE 10
Tumor volume

| | Tumor Volume (mm³) (mean ± SEM) | | | |
|---|---|---|---|---|
| Days after | Vehicle + | Anti-mPD-1 + | muAb1 + | Anti- |
| start of | Rat IgG 2a | Rat IgG2a | Rat IgG2a | mPD-1 + |
| treatment | Isotype | Isotype | Isotype | muAb1 |
| 0 | 81 ± 6 | 81 ± 6 | 81 ± 6 | 81 ± 6 |
| 2 | 117 ± 7 | 114 ± 14 | 120 ± 10 | 108 ± 15 |
| 4 | 195 ± 22 | 143 ± 27 | 171 ± 21 | 115 ± 23 |
| 7 | 376 ± 64 | 175 ± 58 | 252 ± 41 | 84 ± 33 |
| 9 | 560 ± 107 | 231 ± 69 | 363 ± 60 | 75 ± 34 |
| 11 | 704 ± 141 | 219 ± 68 | 522 ± 86 | 74 ± 39 |
| 14 | 1410 ± 317 | 274 ± 90 | 1122 ± 206 | 81 ± 57 |
| 16 | 1486 ± 402 | 330 ± 111 | 1570 ± 334 | 72 ± 57 |
| 18 | 940 ± 367 | 445 ± 150 | 1856 ± 433 | 92 ± 78 |
| 21 | 1153 ± 123 | 628 ± 219 | 1918 ± 541 | 130 ± 102 |
| 23 | 1363 ± 235 | 836 ± 305 | 1986 ± 560 | 176 ± 136 |
| 25 | 1990 ± 423 | 612 ± 300 | — | 310 ± 246 |
| 28 | 2344 ± 535 | 527 ± 309 | — | 124 ± 124 |
| 30 | — | 301 ± 219 | — | 171 ± 171 |
| 32 | — | 419 ± 280 | — | 295 ± 295 |
| 35 | — | 675 ± 420 | — | 0 ± 0 |
| 37 | — | 406 ± 321 | — | 0 ± 0 |
| 39 | — | 621 ± 517 | — | 0 ± 0 |
| 42 | — | 147 ± 95 | — | 0 ± 0 |
| 46 | — | 257 ± 196 | — | 0 ± 0 |
| 53 | — | 917 ± 822 | — | 0 ± 0 |
| 60 | — | 167 ± 167 | — | 0 ± 0 |
| 67 | — | 355 ± 355 | — | 0 ± 0 |
| 74 | — | 686 ± 686 | — | 0 ± 0 |

### Tumor Growth Curves

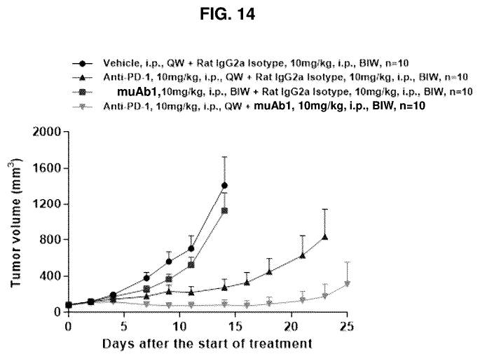

Tumor growth curves are shown in FIG. 14.

### Tumor Growth Inhibition Analysis

Tumor growth inhibition analysis results are shown in Table 11.

TABLE 11
Tumor growth inhibition calculation for muAb1 in combination with anti-
mPD1 therapy in murine subcutaneous GL261 tumor model (female C57BL/6
mice) calculated based on tumor volume measurements on day 14.

| | Tumor Size | | | p value^{d} |
|---|---|---|---|---|
| | (mm³)^{a} at | T/C^{b} | TGI %^{c} | One-way |
| Treatment | day 14 | (%) | (%) | ANOVA |
| Vehicle, i.p., QWx3W + Rat IgG2a Isotype, | 1410 ± 317 | — | — | — |
| 10 mg/kg, i.p., BIWx3W | | | | |
| Anti-PD-1, 10 mg/kg, i.p., QWx3W + | 274 ± 90 | 19.4 | 85.5 | 0.0255(*) |
| Rat IgG2a Isotype, 10 mg/kg, i.p., BIWx3W | | | | |
| muAb1, 10 mg/kg, i.p., BIWx3W + | 1122 ± 206 | 79.6 | 21.7 | 0.8701(ns) |
| Rat IgG2a Isotype, 10 mg/kg, i.p., BIWx3W | | | | |
| Anti-PD-1, 10 mg/kg, i.p., QWx3W + | 81 ± 57 | 5.7 | 100.0 | 0.0100(*) |
| muAb1, 10 mg/kg, i.p., BIWx3W | | | | |

QW = once a week; BIW = twice a week, 3W = 3 weeks; i.p. = intraperitoneal injection.
^{a}Mean ± SEM.
^{b}Tumor Growth Inhibition is calculated by dividing the group average tumor volume for the treated group by the group average tumor volume for the control group (T/C).
^{c}TGI (%) = [1 − (T₁₄ − T₀)/(V₁₄ − V₀)] × 100; T₁₄ is the average tumor volume of a treatment group on day 14, T₀ is the average tumor volume of the treatment group on day 0 after treatment, V₁₄ is the average tumor volume of the vehicle control group on the same day with T₁₄, and V₀ is the average tumor volume of the vehicle group on day 0 after treatment.
^{d}All data were analyzed using Prism 8.0.1 Comparisons between groups were carried out with Games-Howell when homogeneity of variances was not assumed (p = 0.0003(***)).

### Survival Curve

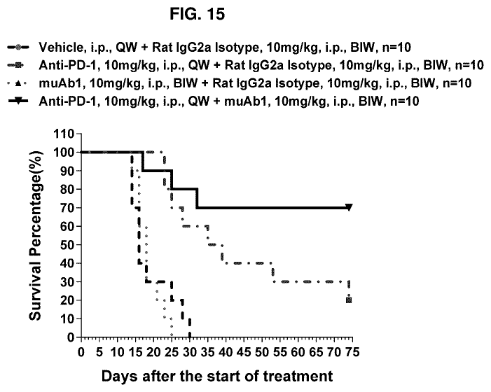

Survival curve results are shown in FIG. 15.

### Kaplan-Meier Survival Analysis

Kaplan-Meier survival analysis results are shown in Table 12.

TABLE 12
Kaplan-Meier survival analysis for muAb1 in combination
with anti-mPD-1 in murine subcutaneous GL261 tumor model
calculated based on tumor volume measurements on day 74.

| | Number of | Median | P value for |
|---|---|---|---|
| | tumor-cured | survival, | Log-rank |
| Treatment | mice | days | test ^{a} |
| Vehicle, i.p., QWx3W + Rat IgG2a Isotype, | — | 16.0 | — |
| 10 mg/kg, i.p., BIWx3W | | | |
| Anti-PD-1, 10 mg/kg, i.p., QWx3W + | 2/10 | 37.0 | 0.0009(***) |
| Rat IgG2a Isotype, 10 mg/kg, i.p., BIWx3W | | | |
| muAb1, 10 mg/kg, i.p., BIWx3W + | 0/10 | 18.0 | 0.6450(ns) |
| Rat IgG2a Isotype, 10 mg/kg, i.p., BIWx3W | | | |
| Anti-PD-1, 10 mg/kg, i.p., QWx3W + | 7/10 | Undefined | 0.0001(***) |
| muAb1, 10 mg/kg, i.p., BIWx3W | | | |

QW = once a week; BIW = twice a week, 3W = 3 weeks; i.p. = intraperitoneal injection.
^{a} All data were analyzed using GraphPad Prism 8.0. Comparisons between groups were carried out with Log-rank test (compared with control group).

Overall, the therapeutic efficacy of muAb1 in combination with anti-mPD1 treatment in murine GL261 tumor model in C57BL/6 mice was evaluated. The results of tumor sizes in different groups post the start of treatment are shown in Tables 10 and 11 and FIG. 14. The mean tumor size of control group (Vehicle+Rat IgG2a Isotype, 10 mg/kg) reached 1410 mm³ on day 14 after the start of treatment. Mono treatment of muAb1 (10 mg/kg) and Rat IgG2a Isotype (10 mg/kg) produced minor antitumor activities with a mean tumor size of 1122 mm³ at the same time (TGI=21.7%; T/C=79.6%; p=0.8701 compared with the control group). Mono administration of anti-mPD-1 (10 mg/kg) produced significant antitumor activities with a mean tumor size of 274 mm³ (TGI=85.5%, T/C=19.4%; p=0.0255). Combination treatments of anti-mPD-1 (10 mg/kg) with muAb1 (10 mg/kg) resulted in the strongest antitumor activities, with a mean tumor size of 81 mm³ at the same time (TGI=100.0%; T/C=19.4% 5.7%; p=0.0100 compared with the control group).

In summary, the therapeutic efficacy of muAb1 (10 mg/kg) in combination with anti-mPD-1 (10 mg/kg) showed significant antitumor activity in a murine subcutaneous GL261 tumor model. In addition to inhibiting tumor growth as mentioned above, combination therapy also improved the overall survival time. The median survival days increased from 18 days for muAb1 and 37 days for anti-PD-1 treatment group, to greater than 75 days for the combination therapy (p=0.0001 compared with the control group).

### Example 20. Pharmacodynamics of an Anti-αvβ8 Antibody in a Pre-Clinical Glioblastoma Cancer Model

The following study was performed as described in Example 18 to obtain tumor and serum samples treated with muAb1 and isotype control for the analysis of TGFb, VEGF and pSMAD2/3. Female C57BL/6 mice were inoculated subcutaneously with 0.5×10⁶ GL261 tumor cells. When tumors reached 79 mm³, mice were randomized and treated by intraperitoneal injection (n=5 per treatment) with 10 mg/kg isotype control (twice weekly for 2 weeks) or 10 mg/kg muAb1 (twice weekly for 2 weeks).

### Mortality, Morbidity, and Body Weight Gain or Loss

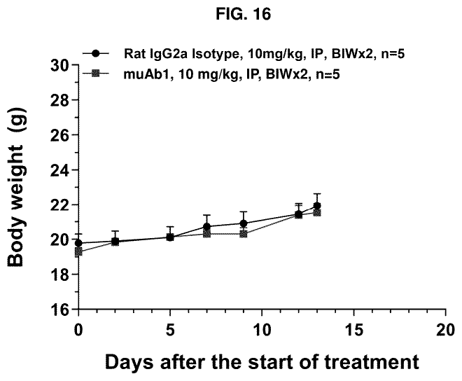

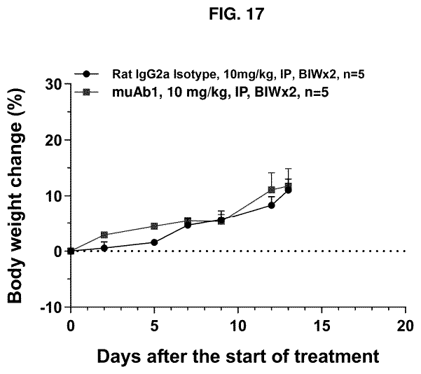

Animal body weight was monitored regularly as an indirect measure of toxicity. In the PD study, none of the animals died, or exhibited abnormal behavior during the study. Body weight changes in female C57BL/6 mice bearing GL261 established tumors dosed with muAb1 is shown in FIGS. 16 and 17.

### Tumor Volume

Mean tumor volume over time in female C57BL/6 mice bearing GL261 established tumors dosed with muAb1 in the PD study is shown in Table 13.

TABLE 13
Tumor volume

Tumor Volume (mm³) (mean ± SEM)

| Days after the start | Rat IgG 2a | Anti-mPD-1 + |
|---|---|---|
| of treatment | Isotype + muAb1 | Rat IgG2a Isotype |
| 0 | 80 ± 10 | 79 ± 8 |
| 2 | 132 ± 21 | 114 ± 13 |
| 5 | 227 ± 42 | 195 ± 47 |
| 7 | 391 ± 86 | 330 ± 113 |
| 9 | 595 ± 136 | 617 ± 226 |
| 12 | 1233 ± 291 | 1615 ± 641 |
| 13 | 1667 ± 397 | 1939 ± 768 |

### Tumor Growth Curves

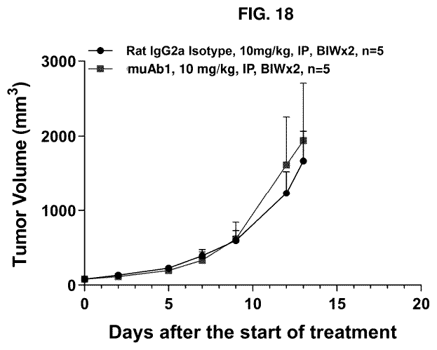

Tumor growth curves are shown in FIG. 18.

### Tumor Growth Inhibition Analysis

Tumor growth inhibition analysis results are shown in Table 14.

TABLE 14
Tumor growth inhibition calculation for muAb1 mono therapy
in murine subcutaneous GL261 tumor model (female C57BL/6 mice)
calculated based on tumor volume measurements on day 13

| | Tumor Size | T/C^{b} | TGI | p value^{c} |
|---|---|---|---|---|
| Treatment | (mm³) ^{a} | (%) | (%) | (T-test) |
| Rat lgG2a Isotype, 10 mg/kg, | 1667 ± 397 | — | — | — |
| IP, BIWx2W | | | | |
| muAb1, 10 mg/kg, IP, BIWx2W | 1939 ± 768 | 116.3 | −17.2 | 0.7611 |

^{a} Mean ± SEM.
^{b}Tumor Growth Inhibition was calculated by dividing the group average tumor volume of the treated group by the group average tumor volume of the vehicle control group (T/C).
^{c}p value calculated based on tumor size.

### Pharmacodynamics

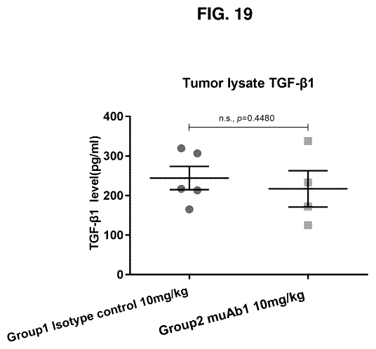

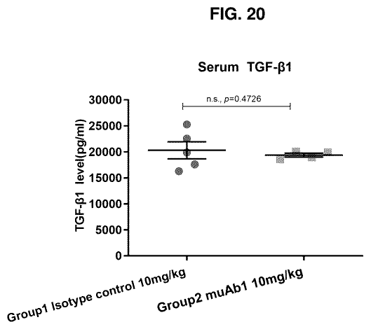

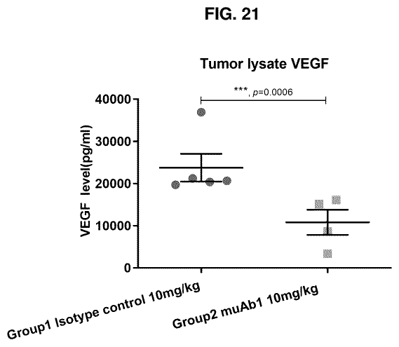

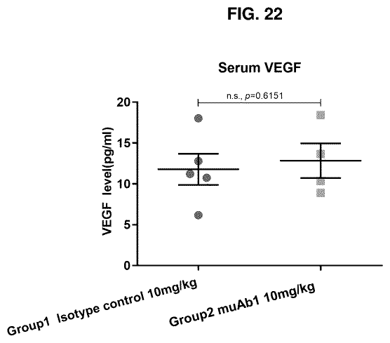

TGF-β1 and VEGF ELISA assays were performed according to manufacturer instructions (Table 15). Results from an ELISA assay determination of TGF-β1 in tumor lysate and serum collected from GL261 tumor-bearing C57BL/6 mice are shown in FIGS. 19-22. VEGF decreased in tumor lysate collected from muAb1 10 mg/kg mono therapy group. There was no significant difference between serum and tumor samples collected from Isotype control group and muAb1 10 mg/kg mono therapy group when detecting TGF-β1. Also, no significant difference was observed in serum samples when testing VEGF.

TABLE 15
Information of ELISA kits

| | Reagent | Vendor | Catalog # |
|---|---|---|---|
| 1 | TGF-β1 | R&D | DB100C |
| 2 | VEGF | R&D | MMV00 |

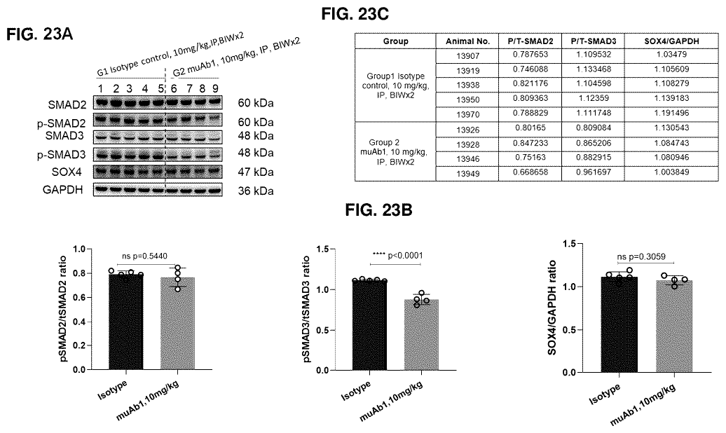

Results from the immunoblotting assay of determination of p/tSmad2, p/tSmad3, Sox4 in tumor lysate collected from GL261 tumor-bearing C57BL/6 mice are shown in FIGS. 23A-23C.

Information for antibodies used in immunoblotting analysis is provided in Table 16.

TABLE 16
Antibodies used in the immunoblotting assays.

| | | | | | Dilution |
|---|---|---|---|---|---|
| | Reagent | Vendor | Host | Catalog # | ratio |
| 1 | SMAD2 | CST | Rabbit | CST-5339 | 1:1000 |
| 2 | pSMAD2 | CST | Rabbit | CST-3108 | 1:1000 |
| 3 | SMAD3 | Abcam | Rabbit | ab40854 | 1:1000 |
| 4 | pSMAD3 | Abcam | Rabbit | ab52903 | 1:2000 |
| 5 | Sox4 | Abcam | Rabbit | ab70598 | 1:1000 |
| 6 | GAPDH | CST | Rabbit | CST-5174 | 1:1000 |
| 7 | Goat anti- | Thermo | Anti-Rabbit | 31462 | 1:10000 |
| | Rabbit IgG-HRP | Fisher | secondary | | |
| | | | antibody | | |

There were no significant changes in pSmad2 and SoX4 expression at the dose of 10 mg/kg muAb1. Expression of pSmad3 decreased in the muAb1 10 mg/kg mono therapy group when compared with the isotype control group.

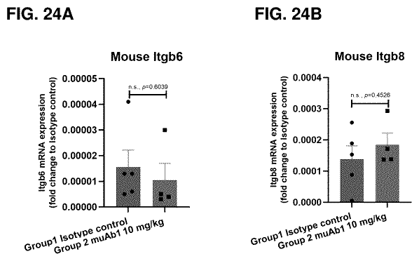

Results from real-time quantitative PCR analysis of avb8 and avb6 expression in tumor samples collected from GL261 tumor-bearing C571BL/6 are shown in FIGS. 24A-24B.

In summary, the pharmacodynamics of muAb1 in murine GL261 tumor model in C571BL/6 mice was evaluated. The results showed decreased tumor VEGF and phosphorylated Smad3 in muAb1 mono therapy group when compared with isotype control group.

Example 21. Evaluation of the Tumor Infiltrating Leukocytes and Peripheral Immune Cells by Flow Cytometry after Treatment with muAb1 as an Single Agent or Combined with Anti-mPD1 (RMP1-14) Antibody in a MC38 Mouse Colorectal Cancer Syngeneic Model in Female C57BL/6 Mice

The objective of this study was to evaluate the avb8 receptor occupancy and T cell subsets, natural killer cells (NK), macrophage and MDSCs population in blood and infiltrating into the tumor by flow cytometry. Expression of PD-1 and Ki67 in lymphocytes subsets, and PD-L1 on tumor cells in MC38 syngeneic model in female C571BL/6 mice were also evaluated. This example provides the general experimental protocols for this study. Example 22 provides the results of this study.

Experimental Design: The in vivo study design is provided in Table 17.

TABLE 17
In vivo study design. IP = intraperitoneal administration.

| | | | | | N for FCM | N for FCM | of Blood in |
|---|---|---|---|---|---|---|---|
| | | | | | of Tumor | of Tumor in | N for FCM |
| Group | Treatment | Dose | Schedule | N | in P1 | P2 | P3 |
| 1 | Isotype | 10 mg/kg, 5 ul/g | IP, Days 0, 6 | 10 | 10 | 10 | 5 |
| | Control | | | | | | |
| 2 | anti mPD-1 | 10 mg/kg, 5 ul/g | IP, Days 0, 6 | 12 | 10 | 10 | 5 |
| 3 | muAb1 + | 10 mg/kg, | IP, Days 0, 3, | 10 | 10 | 10 | 5 |
| | Isotype | 5 ul/g + 10 mg/kg, | 6 + IP, | | | | |
| | Control | 5 ul/g | Days 0, 6 | | | | |
| 4 | muAb1 + anti | 10 mg/kg, 5 ul/g + | IP, Days 0, 3, | 18 | 12 | 11 | 5 |
| | mPD-1 | 10 mg/kg, 5 ul/g | 6 + IP, | | | | |
| | | | Days 0, 6 | | | | |

Tables 18-20 provide the fluorescence-activated cell sorting design for panels 1-3, respectively.

TABLE 18
Panel 1: Design for FACS (tumor_major immune subtype)

| | | | FMO^{[1]} for each |
|---|---|---|---|
| # | Channel | Panel 1 | antibody/fluorophore |
| 1 | Zombie NIR | Live/Dead | — |
| 2 | BV750 | CD45 | CD45 |
| 3 | APC-Cy7 | CD3 | CD3 |
| 4 | APC-Cy5.5 | CD4 | CD4 |
| 5 | Pacific Orange | CD8 | CD8 |
| 6 | PE-Cy5 | CD62L | CD62L |
| 7 | BV570 | CD44 | CD44 |
| 8 | PE-Cy7 | Foxp3 | Foxp3 |
| 9 | BV650 | CD25 | CD25 |
| 10 | PE/Dazzle594 | CD335 | CD335 |
| 11 | BV711 | CD19 | CD19 |
| 12 | Alexa Fluor 532 | CD11b | CD11b |
| 13 | BV785 | Ly6C | Ly6C |
| 14 | BV510 | Ly6G | Ly6G |
| 15 | BB700 | F4/80 | F4/80 |
| 16 | Alexa Fluor 700 | CD206 | CD206 |
| 17 | BV605 | MHCII | MHCII |
| 18 | Alexa Fluor 488 | CD11c | CD11c |
| 19 | PerCP-eF710 | PD-1 | PD-1 |
| 20 | AF647 | GranzymeB | GranzymeB |
| 21 | BV480 | Ki67 | Ki67 |
| 22 | BV421 | TCF-1 | TCF-1 |
| 23 | PE | TIM-3 | TIM-3 |

^{[1]}FMO (Fluorescence minus one) is a control for panel. FMO control consists of all antibodies except for one fluorophore.

TABLE 19
Panel 2: Design for FACS (tumor_T cell effector)

| | | | FMO^{[1]} for each |
|---|---|---|---|
| # | Channel | Panel 2 | antibody/fluorophore |
| 1 | Zombie NIR | Live/Dead | — |
| 2 | BV750 | CD45 | CD45 |
| 3 | APC-Cy7 | CD3 | CD3 |
| 4 | APC-Cy5.5 | CD4 | CD4 |
| 5 | FITC | CD8 | CD8 |
| 6 | BV605 | IL-2 | IL-2 |
| 7 | BV786 | IFN-g | IFN-g |
| 8 | PE-Cy7 | TNF-a | TNF-a |
| 9 | AF647 | Granzyme B | Granzyme B |
| 10 | BV480 | Ki67 | Ki67 |
| 11 | PE | H-2Kb MuLV p15E | H-2Kb MuLV p15E |
| | | Tetramer | Tetramer |
| 12 | PE/Dazzle594 | CD335 | CD335 |

Cell Stimulation Cocktail stimulation for 4 hours
^{[1]}FMO (Fluorescence minus one) is a control for panel. FMO control consists of all antibodies except for one fluorophore.

TABLE 20
Panel 3: Design for FACS (blood_major immune subtype)

| | | | FMO^{[1]} for each |
|---|---|---|---|
| # | Channel | Panel 3 | antibody/fluorophore |
| 1 | Zombie NIR | Live/Dead | — |
| 2 | BV750 | CD45 | CD45 |
| 3 | APC-Cy7 | CD3 | CD3 |
| 4 | APC-Cy5.5 | CD4 | CD4 |
| 5 | Pacific Orange | CD8 | CD8 |
| 6 | PE-Cy5 | CD62L | CD62L |
| 7 | BV570 | CD44 | CD44 |
| 8 | PE-Cy7 | Foxp3 | Foxp3 |
| 9 | BV650 | CD25 | CD25 |
| 10 | PE/Dazzle594 | CD335 | CD335 |
| 11 | BV711 | CD19 | CD19 |
| 12 | Alexa Fluor 532 | CD11b | CD11b |
| 13 | BV785 | Ly6C | Ly6C |
| 14 | BV510 | Ly6G | Ly6G |
| 15 | BB700 | F4/80 | F4/80 |
| 16 | Alexa Fluor 700 | CD206 | CD206 |
| 17 | BV605 | MHCII | MHCII |
| 18 | Alexa Fluor 488 | CD11c | CD11c |
| 19 | PerCP-eF710 | PD-1 | PD-1 |
| 20 | AF647 | GranzymeB | GranzymeB |
| 21 | BV480 | Ki67 | Ki67 |

^{[1]}FMO (Fluorescence minus one) is a control for panel. FMO control consists of all antibodies except for one fluorophore.

Table 21 provides antibodies used in this study.

TABLE 21
Antibodies used in this study.

| # | Fluorochrome | Antibody | Supplier | Cat# |
|---|---|---|---|---|
| 1 | Zombie NIR | Live/Dead | BioLegend | 423106 |
| 2 | BV750 | CD45 | BioLegend | 103157 |
| 3 | APC-Cy7 | CD3 | BioLegend | 100222 |
| 4 | APC-Cy5.5 | CD4 | ThermoFisher | MCD0419 |
| 5 | Pacific Orange | CD8 | ThermoFisher | MCD0830 |
| 6 | PE-Cy5 | CD62L | BioLegend | 104410 |
| 7 | BV570 | CD44 | BioLegend | 103037 |
| 8 | PE-Cy7 | Foxp3 | eBioscience | 25-5773-82 |
| 9 | BV650 | CD25 | BioLegend | 102037 |
| 10 | PE/Dazzle594 | CD335 | BioLegend | 137630 |
| 11 | BV711 | CD19 | BioLegend | 115555 |
| 12 | Alexa Fluor 532 | CD11b | ThermoFisher | 58-0112-82 |
| 13 | BV785 | Ly6C | BioLegend | 128041 |
| 14 | BV510 | Ly6G | BioLegend | 127633 |
| 15 | BB700 | F4/80 | BD | 746070 |
| 16 | Alexa Fluor 700 | CD206 | BioLegend | 141734 |
| 17 | BV605 | MHCII | BioLegend | 107639 |
| 18 | Alexa Fluor 488 | CD11c | BioLegend | 117311 |
| 19 | PerCP-eF710 | PD-1 | eBioscience | 46-9985-82 |
| 20 | AF647 | GranzymeB | BioLegend | 515406 |
| 21 | BV480 | Ki67 | BD | 566109 |
| 22 | BV421 | TCF-1 | BD | 566692 |
| 23 | PE | TIM-3 | eBioscience | 12-5870-82 |
| 24 | FITC | CD8 | MBL | K0227-4 |
| 25 | BV605 | IL-2 | BioLegend | 503829 |
| 26 | BV786 | IFN-g | BD | 563773 |
| 27 | PE-Cy7 | TNF-alpha | BioLegend | 506323 |
| 28 | PE | H-2Kb MuLV | MBL | TS-M507-1 |
| | | p15E Tetramer | | |

Sample Collection: In this study, MC38 tumors were measured on days 0, 2, 5, 7, 9, and 10 after treatment start in two dimensions using a caliper, and the volume was expressed in mm³ using the formula: V=0.5 a×b² where a and b are the long and short diameters of the tumor, respectively. MC38 tumor samples and murine blood samples were collected on day 10. The in vivo study design is shown in the following Table 22.

TABLE 22
Description of experimental design. i.p. = intraperitoneal administration.

| | | | | Dose | Dosing | |
|---|---|---|---|---|---|---|
| Group | n | Treatment | Dose (mg/kg) | Volume | Route | Schedule |
| 1 | 10 | Vehicle + Isotype | 0 + 10 | 5 μL/g | i.p. | Days 0, 6 |
| | | Control | | | | |
| 2 | 12 | Vehicle + | 0 + 10 | 5 μL/g | i.p. | Days 0, 6 |
| | | anti-mPD-1 | | | | |
| 3 | 10 | muAb1 + Isotype | 10 + 10 | 5 μL/g | i.p. | Days 0, |
| | | Control | | | | 3, 6 + |
| | | | | | | Days 0, 6 |
| 4 | 18 | muAb1 + | 10 + 10 | 5 μL/g | i.p. | Days 0, |
| | | anti-mPD-1 | | | | 3, 6 + |
| | | | | | | Days 0, 6 |

Preparation of Single Cell Suspension: For tumors, a freshly collected tumor sample from each mouse was minced separately, and digested with mixed enzymes in C tubes. C tubes were attached onto the sleeve of gentle MACS dissociator before running the program m_imptumor_01_01 once. C tubes were incubated for 30 min at 37° C., followed by another round of program m_imptumor_01_01. Digested tissues were filtered through 70 μm cell strainers. Cells were washed twice with DPBS before staining. For blood, 19×Volume of 1×Red Blood Cell Lysis Solution was added to blood cells and incubated for 2 min at RT. Lysis was repeated for another 2 minutes after centrifugation. Cells were pelleted by centrifugation and washed twice with DPBS before staining.

Stimulation: For panel 2, 1.5×10⁶ cells from each sample were suspended with 200 μL RPMI 1640 medium, which contains Cell Stimulation Cocktail(1*), FBS and Antibiotic-Antimycotic. After incubation for 4 hours at 37° C., cells were washed once with DPBS.

Antibody Staining: For antibody extracellular staining for panels 1 and 3, cells were suspended with 100 μL DPBS containing 0.15 μl Live/Dead and 3 μl purified rat anti-mouse CD16/CD32 antibody per well and incubated at 4° C. in dark for 15 minutes. Extracellular antibody was added and incubated at 4° C. for 30 minutes in the dark. For antibody extracellular staining for panel 2, cells were suspended with 50 μL DPBS containing 1.5 μL purified rat anti-Mouse CD16/CD32 and incubated for 15 minutes at 4° C. in the dark. Then, 5 μL Tetramer was added per well and incubated for 30 minutes at 4° C. in the dark. Next, 50 μL DPBS with 0.15 μL Live/Dead was added per well and incubated for 5 minutes at 4° C. in the dark, followed by addition of extra cellular antibody and incubation at 4° C. for 30 minutes in the dark. After incubation with extracellular antibodies, cells were washed twice with DPBS, fixed and permeabilized for 30 minutes. The cells were stained according to the procedures indicated in specific intracellular antibodies specification. Next, cells were washed twice and suspended with 300 μL staining buffer for detection. About 1×10⁶ live cells per tube were used.

Flow cytometry detection: All samples were detected by Cytek Aurora 5 L Flow Cytometer. Usually, >5,000 CD45+ cells were recorded for further analysis.

Statistical Analysis: All the Flow Cytometry data were analyzed by Flowjo V10 software, Graphpad Prism, SPSS and Excel. The data analyzed included:

For Panel 1:
1. Frequency of Myeloid, T, B, NK, NKT in CD45+ live cells in tumor.
2. Frequency of CD4T, CD8T and its subtypes, include Treg, Effector memory T (TEM), Central memory T (TCM), and naive T in CD45+ live cells in tumor.
3. Frequency of Myeloid subpopulations, include CD11b+DC, Macrophage, PMN-MDSC, M-MDSC, M1 and M2 in CD45+ live cells in tumor.
4. Positive rate and Median Fluorescence Intensity (MFI) of PD-1, Granzyme B, Ki67, TCF-1, TIM-3 in NK, NKT, T and its subtypes in tumor.
5. Ratio of CD4Teff/Treg, CD8T/Treg and M1/M2 in tumor.

For Panel 2:
1. Frequency of MuLV p15E specific CD8T in CD8T or CD45+ live cells in stimulated tumor.
2. Positive rate and Median Fluorescence Intensity (MFI) of IL-2, IFN-g, TNF-a, Granzyme B, Ki67 in NK, T and its subtypes in stimulated tumor.

For Panel 3:
1. Frequency of Myeloid, T, B, NK, NKT in CD45+ live cells in blood.
2. Frequency of CD4T, CD8T and its subtypes, include Treg, Effector memory T (TEM), Central memory T (TCM), and naive T in CD45+ live cells in blood.
3. Frequency of Myeloid subpopulations, include CD11 b+DC, F4/80+ cells, Granulocytes, Monocytes, F4/80+MHCII+ cells and F4/80+CD206+ cells in CD45+ live cells in blood.
4. Positive rate and Median Fluorescence Intensity (MFI) of PD-1, Granzyme B, Ki67 in T and its subtypes in blood.
5. Ratio of CD4Teff/Treg and CD8T/Treg in blood.

One-way ANOVA was used for statistical analysis. When a significant F-statistics (a ratio of treatment variance to the error variance) was obtained, comparisons between vehicle and treatment group were carried out with Games-Howell (equal variances not assumed) or Tukey (equal variances assumed) test. All data were analyzed using SPSS 17.0. *, p<0.05; **; p<0.01, **, p<0.001. Error bars represent Standard Error of Mean (SEM).

### Example 22. Immune Cell Populations Increase in Response to Treatment with an Anti-αvβ8 Antibody in a Pre-Clinical Mouse Colorectal Cancer Syngeneic Model

The following study was performed as described in Example 21 to evaluate the avb8 receptor occupancy and T cell subsets, natural killer cells (NK), macrophage and MDSCs population in blood and infiltrating into the tumor by flow cytometry. Expression of PD-1 and Ki67 in lymphocytes subsets, and PD-L1 on tumor cells in MC38 syngeneic model in female C57BL/6 mice were also evaluated.

### Tumor Volume

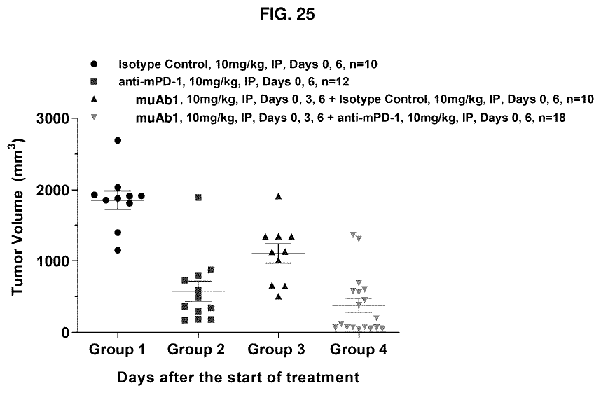

Tumor volume results on Day 10 are shown in FIG. 25.

### Result for FCM Analysis of Tumor for Panel 1

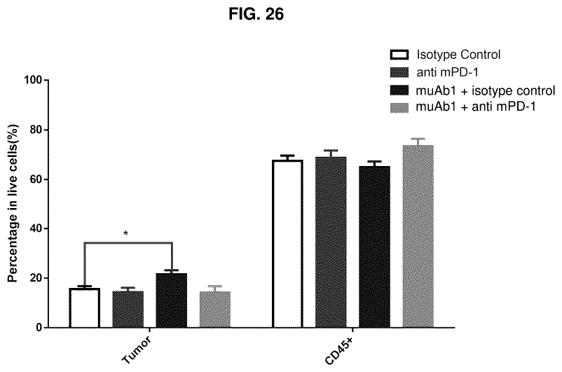

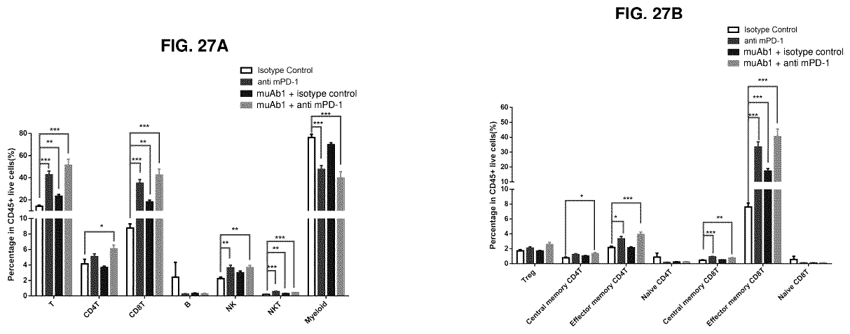

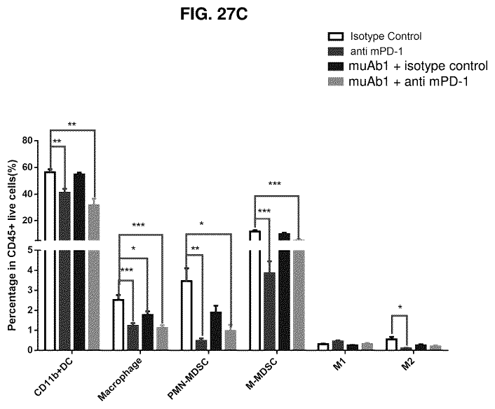

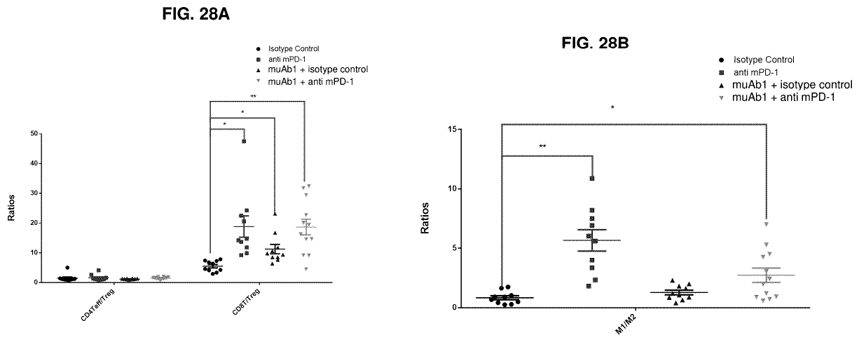

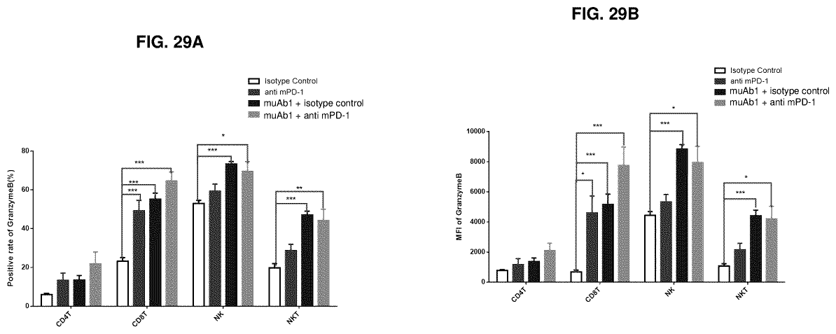

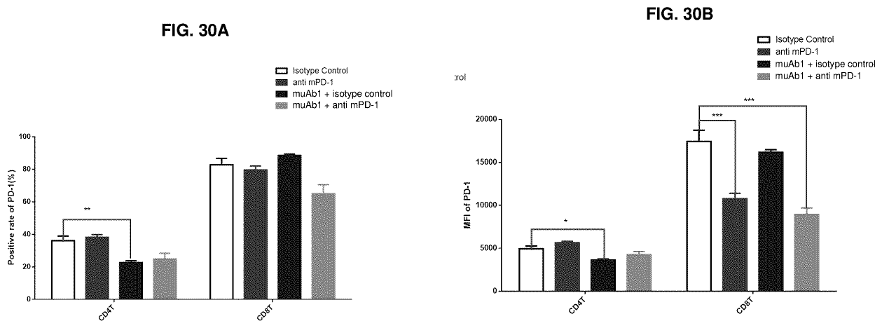

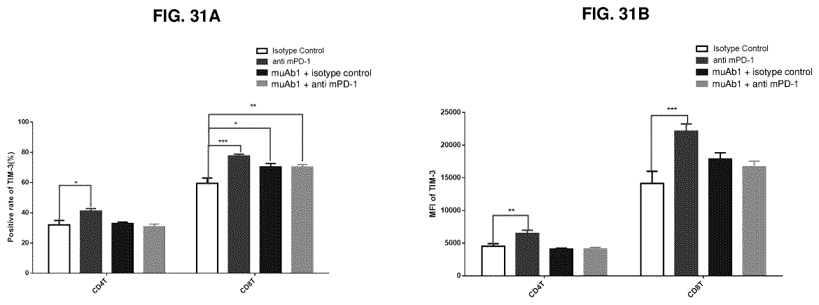

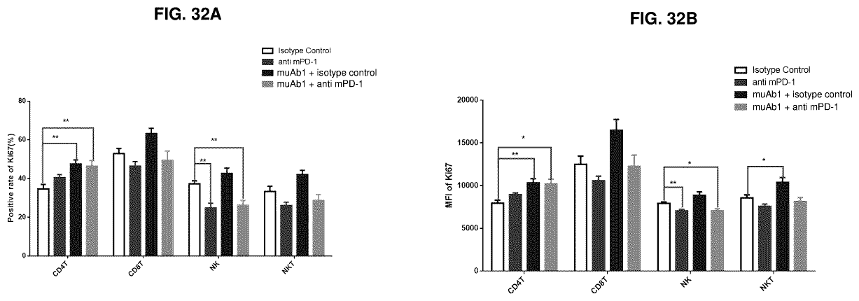

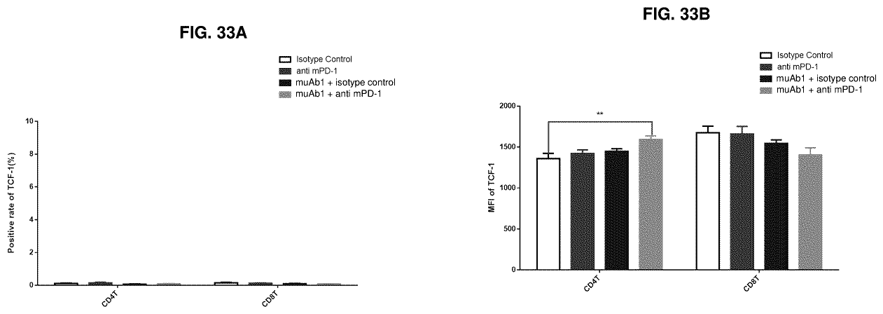

Results of the PD study for Panel 1 are shown in FIGS. 26-33. For tumors in Panel 1, comparing with the isotype control group, the percentage of CD4T, CD4TCM, CD4TEM, Treg, CD8TCM showed increase in PD-1 monotherapy and muAb1+PD-1 combination groups, and the percentage of CD8T, CD8TEM, NK, NKT showed increase in all treatment groups (FIGS. 27A-27C), while the percentage of CD11 b₃₀ DC, PMN-MDSC, M-MDSC, Macrophage, M2 showed a decreased trend in PD-1 monotherapy and muAb1+PD-1 combination groups (FIGS. 27A-27C). The percentage of Macrophage in muAb1 single group also decreased significantly (FIGS. 27A-27C). The ratio of CD8T/Treg and M1/M2 significantly increased in PD-1 monotherapy and muAb1+PD-1 combination groups, and the ratio of CD8T/Treg also increased in the muAb1 monotherapy group (FIGS. 28A-28B). The positive rate or MFI of GranzymeB showed an increased trend in CD4T, CD8T, NK and NKT in all treatment groups. The positive rate or MFI of PD-1 in CD8T showed a decreased trend in PD-1 monotherapy and muAb1+PD-1 combination groups. The positive rate or MFI of TIM-3 in CD8T showed an increased trend in all treatment groups. The positive rate or MFI of Ki67 in CD4T showed an increased trend in all treatment groups, and Ki67 in CD8T, NKT showed an increased trend in the muAb1 monotherapy group (FIGS. 29-33).

### Result for FCM Analysis of Tumor for Panel 2

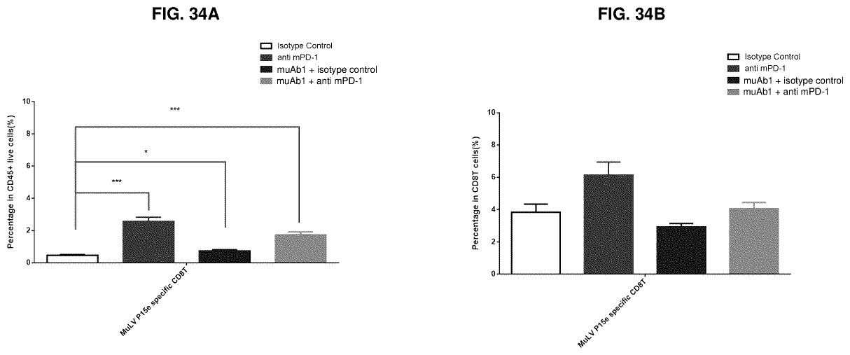

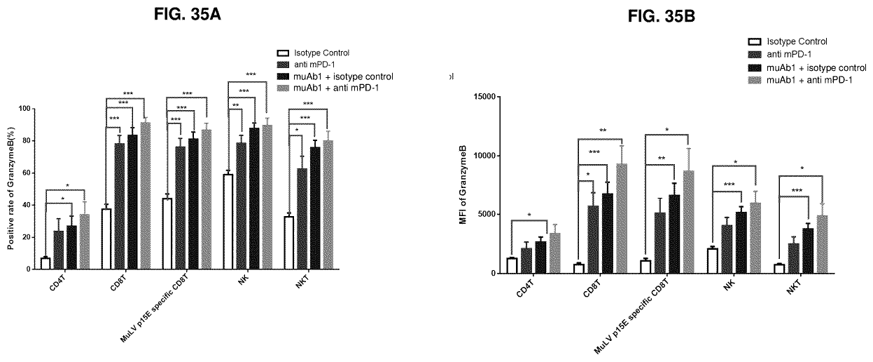

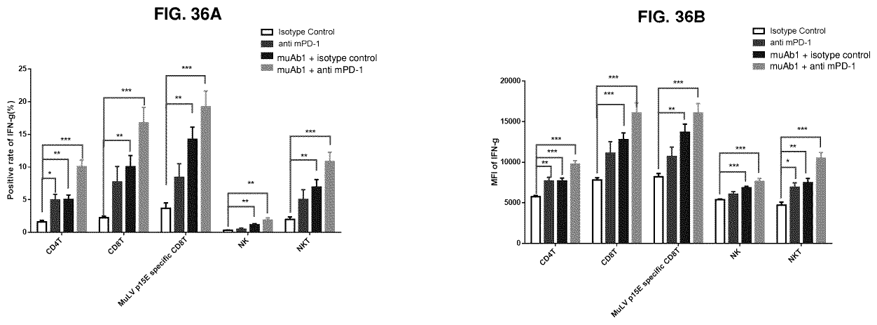

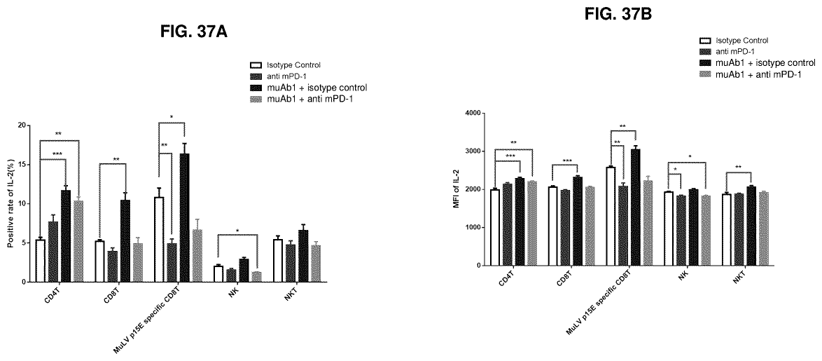

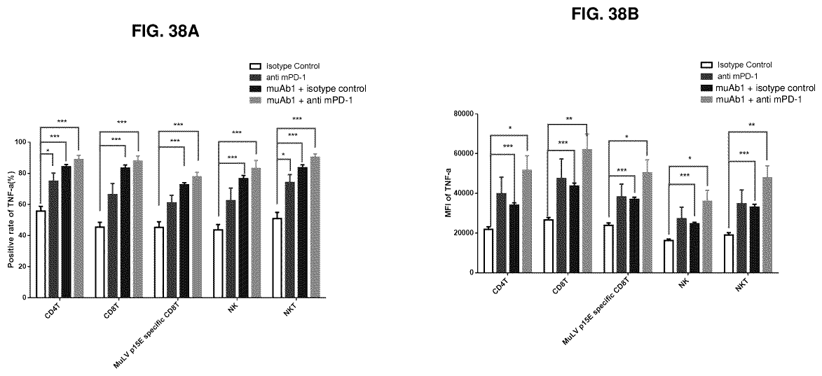

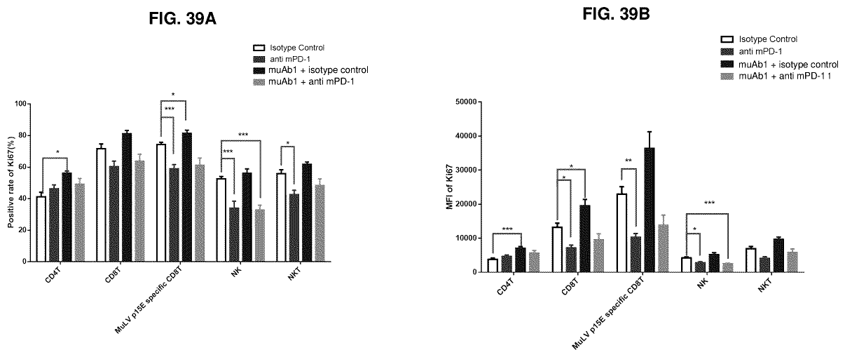

Results of the PD study for Panel 2 are shown in FIGS. 34-39. For stimulated tumor, in Panel 2, comparing with control group, the percentage of MuLV p15E specific CD8T in CD45+ live cells increased in all treatment groups, especially in PD-1 monotherapy and muAb1+PD-1 combination groups (FIGS. 34A-34B). The positive rate or MFI of GranzymeB, IFN-g, TNF-α showed an increased trend in NK, NKT, CD4, CD8T, MuLV p15E specific CD8T of all treatment groups, especially for the combination group. The positive rate or MFI of IL-2 in CD4T increased in all treatment groups, especially for muAb1 monotherapy and combination therapy groups, however, the positive rate or MFI of IL-2 in CD8T, MuLV p15E specific CD8T increased in the muAb1 monotherapy group, and showed a decreased trend in the anti-mPD-1 treatment groups. For Ki67, the positive rate or MFI in CD4T, CD8T and MuLV p15E specific CD8T showed an increased trend in the muAb1 group, but after mPD-1 treatment, the positive rate or MFI of Ki67 decreased in NK, NKT, CD8T, MuLV p15E specific CD8T (FIGS. 35-39).

### Result for FCM Analysis of Blood for Panel 3

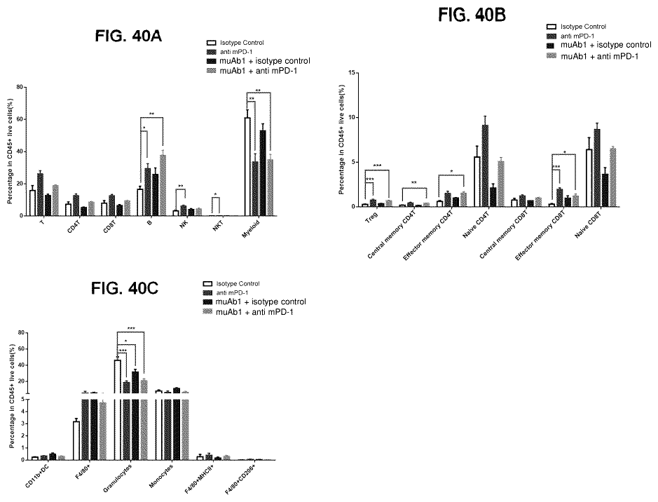

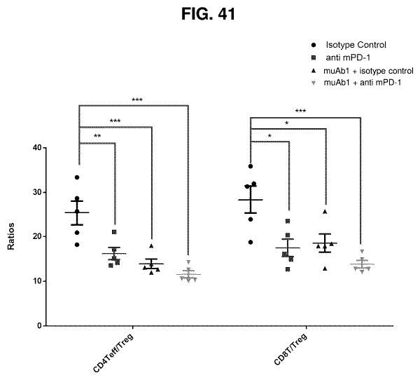

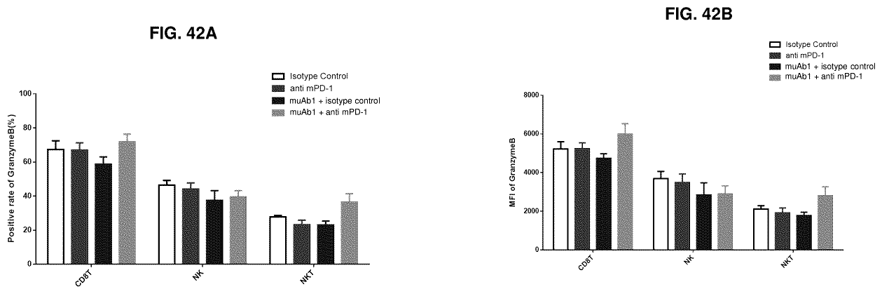

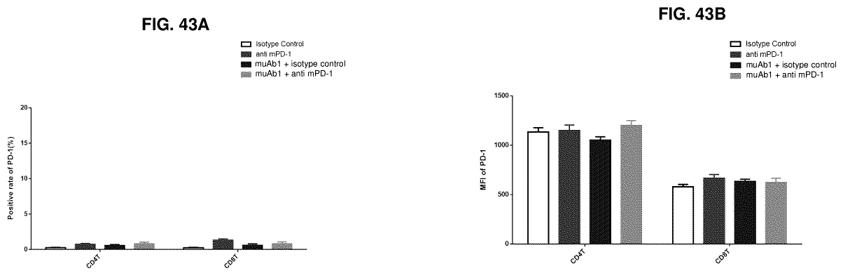

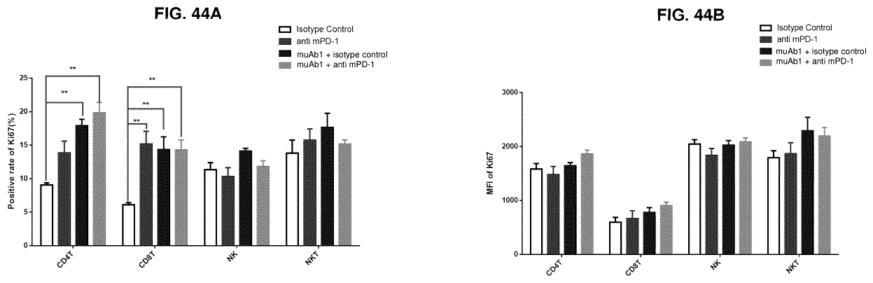

Results of the PD study for Panel 3 are shown in FIGS. 40-44. For blood, comparing with the control group, the percentage of Treg, CD4TCM, CD4TEM, CD8TCM and CD8TEM showed an increased trend in PD-1 and muAb1+PD-1 groups, and the percentage of B, F4/80+ cells increased in all treatment groups, while the percentage of granulocytes decreased in all treatment groups (FIGS. 40A-40C). The ratio of CD4Teff/Treg and CD8T/Treg significantly decreased in all treatment groups (FIG. 40). The positive rate or MFI of Ki67 in CD4T, CD8T showed an increased trend in all treatment groups (FIGS. 40-42).

In summary, various CD45-positive immune cell populations (e.g., effector T-cell populations) increased in the tumor in response to muAb1 treatment. In addition, the rates of Granzyme B, IL2, IFNg, TNFa, and Ki67 positivity were higher across different immune cell types (CD4T, CD8T, NK cells), indicating that the combination of muAb1 and anti-mPD-1 increased the reactivity of these populations. Furthermore, depletion of some immune cell populations in the blood suggests that there has been mobilization from the blood to the tumor of these populations.

### Example 23. Treatment of Human Subjects with an Anti-αvβ8 Antibody

Patients with select relapsed/refractory solid tumors are enrolled in a Phase 1-2, four-part study to evaluate the safety, tolerability, of huAb1. Participants with all histological types of endometrial, ovarian, cervical, non-small cell lung cancer (NSCLC), renal cell cancer (RCC) (e.g., renal clear cell carcinoma or kidney renal papillary cell carcinoma), head and neck squamous cell cancer (HNSCC), melanoma, and triple negative breast cancer (TNBC) are preferred but participants with esophageal, gastric, urothelial, colorectal, and pancreatic cancers are admitted to the study. Mesothelioma and all grades of glioma or glioblastoma are excluded. All participants will have had disease progression (PD) after at least one line of therapy or have no other standard therapy of proven clinical benefit currently available or be recommended based on the investigator's individual risk-benefit assessment for the participant.

In Part A, participants will receive huAb1 monotherapy administered intravenously (i.v.) over 30 (±5) minutes on Day 1 of each 21-day cycle, or on Days 1 and 15 of each 28-day cycle. The dose of huAb1 was either 1 mg/kg, 3 mg/kg, 10 mg/kg, or 30 mg/kg.

In Part B, participants receive huAb1 (a low dose or a high dose) in combination with an additional therapy (a PD-(L)1 inhibitor, e.g., an anti-PD-1 antibody or an anti-PD-L1 antibody; or immune-priming stereotactic body radiation therapy). A cohort of six participants with select solid tumors (any histological type of endometrial, ovarian, cervical, NSCLC, RCC, HNSCC, melanoma, and TNBC) are be treated with huAb1 (three participants at a low-dose, and three participants at a high-dose, as selected in Part A) in combination with a PD-(L)1 inhibitor. An additional cohort of six participants with any histological type of NSCLC or HNSCC are treated with either a low-dose (three participants) or high-dose (three participants) of huAb1 in combination with immune-priming SBRT (3×8 Gy) focused on at least a single soft-tissue lesion (excluding lesions in the brain and bone). The therapy is administered on Day 1 of each 21-day cycle, or on Days 1 and 15 of each 28-day cycle. On each treatment day, huAb1 is administered first, followed by the PD-(L)1 inhibitor or immune-priming SBRT, as applicable.

In Part C, NSCLC participants receive combination treatment consisting of huAb1 in combination with an PD-(L)1 inhibitor, as follows:
huAb1 (low-dose or high-dose) administered by 30-minute i.v. infusion on Day 1 of each 21-day cycle or on Days 1 and 15 of each 28-day cycle; plus
PD-(L)1 inhibitor administered at the recommended dose and schedule
   On each treatment day, huAb1 is administered first, followed by the PD-(L)1 inhibitor.

In Part D, participants are treated with combinations of the RP2D dose of huAb1 with PD-(L)1 inhibitor at the recommended dose and schedule with or without immune priming stereotactic body radiation therapy. It is anticipated that at least one or more of the following cohorts may be selected for investigation:
NSCLC treated with RP2D huAb1, PD-(L)1 inhibitor, and SBRT
HNSCC treated with RP2D huAb1, and PD-(L)1 inhibitor
HNSCC treated with RP2D huAb1, PD-(L)1 inhibitor, and SBRT
Ovarian cancer treated with RP2D huAb1, and PD-(L)1 inhibitor
   On each treatment day, huAb1 is administered first, followed by the PD-(L)1 inhibitor with or without immune priming stereotactic body radiation therapy (3×8 Gy fractions post-infusion on C1 D1 and on C1 D3 and C1 D5 for a total of 24 Gy), as applicable.

### Inclusion Criteria:

Participants are eligible to be included in the study only if all of the following criteria apply:
1. Signed informed consent form (ICF).
2. Male or female participants ≥18 years of age at the time of signing the ICF.
3. Histologically and/or cytologically confirmed diagnosis of select advanced or metastatic solid tumor (relapsed/refractory disease) that has progressed following at least one line of therapy[1] or have no other standard therapy with proven clinical benefit available or recommended based on the investigator's individual risk-benefit assessment for the participant.
   a) Part A: All histological types of the following tumors: endometrial, ovarian, cervical, NSCLC, RCC, HNSCC, melanoma, and TNBC, and in agreement with the sponsor, participants with, esophageal, gastric, urothelial, colorectal, and pancreatic cancer may be enrolled.
   b) Part B: All histological types of the following tumors: endometrial, ovarian, cervical, NSCLC, RCC, HNSCC, melanoma, and TNBC.
   c) Part C: NSCLC (adenocarcinoma or squamous cell histological types)[2].
   d) Part D: NSCLC (adenocarcinoma or squamous cell histological types), HNSCC, ovarian[2].
   [1] Cohorts treated with huAb1 in combination with SBRT will be limited to those tumor types where SBRT is considered feasible as per investigator's team assessment e.g., NSCLC and HNSCC.
   [2] Prior therapy may have included an immune checkpoint inhibitor (ICI), such as a PD-(L)1 inhibitor, or anti cytotoxic T-lymphocyte-associated protein 4 (CTLA4) treatment.
4. Measurable disease on imaging, as assessed by RECIST v1.1 (Eisenhauer, 2009) Note: All participants will need ≥two measurable lesions; participants receiving SBRT will need ≥three measurable lesions.
   Note: for participants with measurable brain metastases at baseline, the Response Evaluation Criteria in Neuro oncology (RANO) criteria (Wen, 2010) will be used for tumor measurements.
5. Eastern Cooperative Oncology Group (ECOG) performance status ≤2.
6. Life expectancy 212 weeks.
7. Adequate hematologic and end-organ function, defined based on the following laboratory results obtained within 14 days prior to C1 D1 (and reconfirmed within 96 hours prior to the first dose of huAb1 to remain eligible):
   a) Absolute neutrophil count ≥1.5×109/L (1500/μL), without granulocyte colony-stimulating factor (G-CSF) support. G-CSF may be administered until 14 days prior to C1 D1.
   b) Platelet count ≥100×109/L (100,000/μL) without transfusion.
   c) Hemoglobin ≥90 g/L (9 g/dL). Participants may be transfused or receive erythropoietic treatment to meet this criterion up until 14 days prior to C1 D1.
   d) For participants not receiving therapeutic anticoagulation: international normalized ratio (INR) and (activated) partial thromboplastin time (aPTT or PTT)≤1.5×upper limit of normal (ULN). Participants receiving therapeutic anticoagulation should be on a stable dose. For participants receiving other oral anticoagulant therapy, aPTT/PTT must be within the intended therapeutic range of the anticoagulant and should be monitored as per local standards.
   e) Aspartate aminotransferase (AST) and alanine aminotransferase (ALT)≤3×ULN (55×ULN for participants with documented liver metastases).
   f) Total bilirubin ≤1.5×ULN≤3×ULN for participants with Gilbert's syndrome).
   g) Estimated glomerular filtration rate >40 mL/min/1.73 m2.
8. Willingness to provide tumor biopsy samples:
   a) Baseline samples (for Parts A-D) should be either a fresh biopsy gathered during the screening period or an archival FFPE tumor sample containing 220% tumor cells obtained within 1 year prior to C1 D1.
   b) On-treatment samples (Parts A-D) will be collected on C2D15 (+5 days) and are considered mandatory unless agreed by the sponsor.
9. For women: post-menopausal or able to comply with the contraceptive requirements of the protocol. For men: agree to contraceptive requirements of the protocol.

### Exclusion Criteria:

Participants are excluded from the study if any of the following criteria apply:

### Medical Conditions:

1. Known metastases to the central nervous system (CNS), unless asymptomatic, radiologically stable (i.e., without evidence of progression for at least 4 weeks by repeat imaging), and without requiring steroids for at least 4 weeks prior to first dose of study intervention (C1D1). Participants with carcinomatous meningitis are also excluded.
   Note: For participants with a history of CNS involvement, or a clinical suspicion of CNS involvement, brain imaging should be performed during screening
2. History of solid tumor malignancy other than the diseases under study, diagnosed within the last
3 years of study enrollment, excluding adequately treated basal cell carcinoma of the skin, squamous cell carcinoma of the skin, or in situ cervical cancer, in situ breast cancer, in situ prostate cancer (participants must have shown no evidence of active disease for 2 years prior to enrollment).
3. History of ascites or pleural effusions, unless successfully treated, asymptomatic, and not requiring treatment for >4 months prior to C1 D1.
4. History of and/or current cardiovascular events or conditions:
   a) History of myocardial infarction, unstable or severe angina, or arterial thrombotic event (such as cerebrovascular attack or transient ischemic attack) within 6 months prior to C1 D1.
   b) Current New York Heart Association Stage II-IV congestive heart failure.
   c) Left ventricular ejection fraction (LVEF)<50%
      Note: LVEF assessment by echocardiogram or multigated acquisition scan performed as part of the participant's regular care within 4 weeks prior to the screening visit may be used for confirmation of eligibility.
   d) Unstable arrhythmia, or history or presence of a clinically significant (in the investigator's opinion) electrocardiogram (ECG) abnormality.
   e) Screening QTc interval (average of triplicate measurements; corrected for heart rate using Fridericia's formula)>480 msec.
   f) Uncontrolled hypertension, defined as a systolic blood pressure >150 mmHg and/or diastolic blood pressure >100 mmHg at screening, despite optimal antihypertensive therapy.
5. Positive tests on screening for the following:
   a) Human immunodeficiency virus (HIV).
   b) Hepatitis B surface antigen (HbsAg).
   c) Hepatitis B core antibody (HbcAb).
      Patients with a positive HbcAb test followed by a negative hepatitis B virus (HBV) deoxyribonucleic acid (DNA) test at screening may be enrolled.
   d) Hepatitis C virus (HCV) antibody test
      Patients with a positive HCV antibody test followed by a negative HCV ribonucleic acid (RNA) test at screening may be enrolled.
6. Active thrombophlebitis, thromboembolism, hypercoagulability states, or uncontrolled bleeding, uncontrolled diabetes.
7. Chronic severe liver disease or liver cirrhosis.
8. Systemic autoimmune disease (e.g., systemic lupus erythematosus, active rheumatoid arthritis, inflammatory bowel disease, ulcerative colitis, Crohn's disease, etc.) with the exception of participants with autoimmune-mediated hypothyroidism well controlled with thyroid-replacement hormone, well controlled eczema, psoriasis, or chronic lichen simplex with only dermatologic manifestations that do not exceed 10% of the body surface area at screening.
9. Interstitial lung disease (such as idiopathic pulmonary fibrosis), or pneumonitis with the exception of radiation pneumonitis (fibrosis) in the radiation field >6 months prior to C1 D1.
   Note: Participants with interstitial lung disease (such as idiopathic pulmonary fibrosis) and participants with a history of chronic pneumonitis (pneumonitis that persists or worsens with steroid tapering, and required ≥12 weeks of steroids/immunosuppression) regardless of the etiology (ICI, radiation, infection, etc.) must be excluded. Participants with a history of pneumonitis that improved or resolved (radiographic response) with 4-6 weeks of corticosteroid therapy >3 months prior to C1 D1 may be enrolled as per the investigator's judgement.
10. Any active or symptomatic persistent infection (bacterial, viral, or fungal) requiring systemic therapy within 14 days prior to C1 D1. A minimum 7-day washout prior to C1 D1 must be observed for any antibiotic use. Participants with clinical/laboratory evidence of persistent infection must be excluded.
11. Any psychiatric illness or social situation that would limit compliance with study requirements.
12. Any other significant co-morbidity, disease, metabolic dysfunction, physical examination, or clinical laboratory finding that contraindicates the use of an investigational drug, or may represent an unacceptable risk from treatment complications, or may affect adherence to the study procedures or the interpretation of the results.

### Ineligible Prior and Ongoing Treatment Requirements:

13. Previous treatment with anti-cancer therapies within the following timeframes:
   a) Cytotoxic chemotherapy, mAbs, and/or small molecule tyrosine kinase inhibitors within 14 days prior to C1 D1, or 5 half-lives, whichever is shorter.
   b) Nitrosourea or mitomycin-C within 42 days prior to C1 D1.
   c) Any investigational cancer treatment within 28 days prior to C1 D1, or 5 half-lives, whichever is shorter.
   Note: participants with advanced prostate cancer who are receiving luteinizing hormone releasing hormone (LHRH) agonists are permitted onto the study and should continue use of these agents during study intervention.
14. Prior organ transplant (including allogeneic bone marrow transplant).
15. Prior therapy with an integrin αvβ8 or transforming growth factor beta (TGFβ) antagonist, such as an antibody, receptor, or kinase inhibitor or anti-sense therapy.
16. Requirement for antibiotic use (therapeutic or prophylactic) within 7 days prior to C1 D1.
17. Any systemic immunosuppressive therapy within 14 days prior to the first dose of study intervention or anticipated during the study.
18. Requirement for systemic corticosteroid therapy at doses higher than the equivalent of prednisolone 10 mg daily for any indication, including replacement therapy for hypoadrenalism, within 7 days prior to the first dose of huAb1.
   Note: Participants receiving inhaled or topical corticosteroids or local steroid injections may be enrolled.
   Note: Participants who have been stabilized on an oral daily dose of prednisolone 510 mg (or equivalent), for at least 7 days prior to the first dose of study intervention may participate.
19. Participants who are on treatment with drugs known to prolong the QT/QTc interval.
20. Live, attenuated vaccine within 28 days prior to the first dose of huAb1, or anticipation of need for such a vaccine during the course of the study (COVID-19 vaccination is acceptable).
21. Any other therapy that is prohibited during the study.

### Other Exclusion Criteria:

22. Known hypersensitivity to the study intervention(s) or any component(s) of the study intervention(s).
23. History of drug-induced anaphylactic or other severe hypersensitivity reactions.
24. History of any of the following: drug-induced severe cutaneous adverse reaction (including, but not limited to Stevens-Johnson syndrome/toxic epidermal necrolysis [SJS/TEN], or drug reaction with eosinophilia and systemic symptoms [DRESS]), or dose-limiting immune-mediated reactions.
25. Pregnant or breast-feeding women.

### Primary Safety Analysis

Safety data, including Aes, laboratory test results, vital signs, and ECGs will be summarized by dose and assessment time points, as appropriate. Change from baseline will be included in summary tables for laboratory, ECG, and vital sign parameters. Shifts in grade from baseline to maximum post-baseline grade will be summarized for each dose level, as appropriate, for applicable laboratory data. Summaries of subjects with Aes Grade 23 or laboratory values Grade 23 will be tabulated by dose level, as appropriate.

### Efficacy Analyses

### Definition of Endpoint(s)

The primary efficacy endpoint (Parts B-D; Safety Lead-in, Dose Optimization and Expansion) is the OSR, defined as objective response rate (ORR) (CR plus PR) plus the rate of participants with SD that lasted for at least 4 months. At each tumor assessment timepoint, objective response will be determined by the investigator according to RECIST v1.1.

Secondary efficacy endpoints are defined as follows:
ORR: defined as the percentage of participants who achieved CR or PR according to RECIST v1.1 during the study
Clinical benefit rate (CBR): defined as the percentage of participants who achieved CR, PR, or SD according to RECIST v1.1 during the study
Progression free survival (PFS): defined as the time from the first dose of study intervention to the first occurrence of PD, as determined by the investigator using RECIST v1.1, or death from any cause during the study, whichever occurs first
TTP: defined as the time from the first dose of study intervention to the first occurrence of PD, as determined by the investigator using RECIST v1.1
OS: defined as the time from the first dose of study intervention to death from any cause
Duration of response (DoR): defined as the period from the date of initial CR or PR until the date of PD or death from any cause during the study, whichever occurs first. DoR will be evaluated in the subset of participants with measurable disease at baseline, who have achieved an objective response according to RECIST v1.1
TTR: defined as the time from the first dose of study treatment to the date of first documented CR or PR according to RECIST v1.1

### Analysis of the Primary Efficacy Endpoint

For the primary efficacy endpoint of OSR, point estimates and 90% exact confidence intervals (Cis) for OSR will be evaluated independently within each cohort. The analysis of the primary efficacy endpoint will be based on tumor response assessments by the investigator, according to RECIST v1.1. A sensitivity analysis of the primary efficacy endpoint in Parts B-D only (i.e., excluding non randomized overall success rates from Part B) may also be performed.

### Analyses of the Secondary Efficacy Endpoints

Secondary efficacy endpoints (ORR, CBR, PFS, TTP, OS, DoR, and TTR) determined according to RECIST will be analyzed for Part A, and Parts B-D separately, as follows:
ORR: Point estimates and 90% exact Cis for ORR will be evaluated independently within each cohort (if sufficient response data is available)
CBR: Point estimates and 90% exact Cis for CBR will be evaluated independently within each cohort
PFS: Kaplan-Meier estimates of median PFS and its associated 95% Cis will be provided. In addition, landmark estimates at 6 months, 12 months, etc. will be provided. Participants alive and progression-free will be censored
TTP: Kaplan-Meier estimates of median TTP and its associated 95% Cis will be provided
OS: Kaplan-Meier estimates of median OS and its associated 95% Cis will be provided. In addition, landmark estimates at 6 months, 12 months, etc. will be provided
DoR: Kaplan-Meier estimates for median DoR and its associated 95% Cis will be provided. DoR will be evaluated in the subset of participants with measurable disease at baseline, who have achieved an objective response according to RECIST v1.1
TTR: Descriptive statistics will be provided for those participants that meet the conditions of TTR Further details, including on handling missing data and censoring will be provided in the SAP.

### Pharmacokinetic Analyses

Data on serum concentrations of huAb1 will be listed by participant and dose within each study part. As appropriate, non-compartmental and/or compartmental methods will be used to calculate PK parameters for huAb1 from the huAb1 serum concentration-time data collected from participants receiving huAb1. Interim analyses may be conducted at completion of a dose cohort, or at intermediate intervals during the study. PK parameters will be summarized where appropriate using following descriptive statistics: N, n, arithmetic mean, arithmetic standard deviation, arithmetic coefficient of variation, median, minimum, maximum, geometric mean, and geometric coefficient of variation. A complete analysis of huAb1 pharmacokinetics will be conducted at the end of the study and described in the SAP. Parameters to evaluate may include the following:
Cmax Maximum observed concentration
Tmax Time during dosing at which Cmax was observed
AUC0-last Area under the concentration-time curve from time 0 to last measurable concentration
AUC0-inf Area under the concentration-time curve from time 0 extrapolated to infinity
Lambda-z (λz or kel) Terminal phase elimination rate constant
T½ Half-life
Vz Volume of distribution based on terminal elimination phase
CL Clearance
Cmax/Dose Cmax divided by the administered dose
AUC0-last/Dose AUC0-last divided by the administered dose

### Pharmacodynamic Analyses

Blood and tumor samples will be assessed for pharmacodynamic activity. For tumor-based huAb1 related biomarkers and αvβ8 expression, intraparticipant comparisons between baseline (archival or fresh) and post-baseline tumor samples (optional fresh samples) will be made if sample quality and quantity permit. Exploratory analysis may be performed to evaluate modulation of biomarkers related to huAb1 mechanism of action and relationship of biomarker/combination of biomarkers with clinical outcome.

### Example 24. Treatment of Advanced Solid Tumors by Administration of an Anti-αvβ8 Antibody as a Monotherapy

According to the methods disclosed herein, a physician of skill in the art can treat a subject, such as a human patient, having a relapsed/refractory solid tumor (e.g., endometrial, ovarian, cervical, lung cancer (e.g., non-small cell lung cancer (NSCLC) or small-cell lung cancer), renal cell carcinoma cancer (RCC) (e.g., renal clear cell carcinoma and kidney renal papillary cell carcinoma), hepatocellular carcinoma, glioblastoma, head and neck cancer (e.g., head and neck squamous cell cancer (HNSCC)), melanoma, brain cancer, optionally gliomas, breast cancer (e.g., triple-negative breast cancer (TNBC)), esophageal, gastric, urothelial, colorectal, bile duct, and pancreatic cancer). The patient will have had disease progression after at least one line of therapy or have no other standard therapy of proven clinical benefit currently available. To treat the subject, a physician of skill in the art can administer to the subject huAb1 in one or more dosing cycles.

HuAb1 is administered to the subject intravenously (e.g., by intravenous infusion over 30±5 minutes) at a dose of about 1 mg/kg to about 30 mg/kg (e.g., about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, or about 25 mg/kg to about 30 mg/kg). HuAb1 is administered on Day 1 of each 21-day dosing cycle (Q3W), or on Days 1 and 15 of each 28-day dosing cycle (Q2W).

Following administration of huAb1 to a patient, a practitioner of skill in the art can monitor the patient's response to the therapy by a variety of methods. For example, a physician can monitor the occurrence of dose-limiting toxicities (DLTs). A finding that the patient exhibits no DLTs following administration of huAb1 indicates that the dose level is safe and tolerable. Subsequent doses can be determined and administered as needed.

### Example 25. Treatment of Solid Tumors by Administration of an Anti-αvβ8 Antibody in a Combination Therapy

According to the methods disclosed herein, a physician of skill in the art can treat a subject, such as a human patient, having a relapsed/refractory solid tumor (e.g., endometrial, ovarian, cervical, lung cancer (e.g., non-small cell lung cancer (NSCLC) or small-cell lung cancer), renal cell carcinoma cancer (RCC) (e.g., renal clear cell carcinoma and kidney renal papillary cell carcinoma), hepatocellular carcinoma, glioblastoma, head and neck cancer (e.g., head and neck squamous cell cancer (HNSCC)), melanoma, brain cancer, optionally gliomas; breast cancer (e.g., triple-negative breast cancer (TNBC)), esophageal, gastric, urothelial, colorectal, bile duct, and pancreatic cancer). The patient will have had disease progression after at least one line of therapy or have no other standard therapy of proven clinical benefit currently available. To treat the subject, a physician of skill in the art can administer to the subject huAb1 in a combination therapy.

HuAb1 may be administered to the subject intravenously (e.g., by intravenous infusion over 30±5 minutes) at a dose of about 1 mg/kg to about 30 mg/kg (e.g., about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, or about 25 mg/kg to about 30 mg/kg). HuAb1 may be administered on Day 1 of each 21-day dosing cycle (Q3W), or on Days 1 and 15 of each 28-day dosing cycle (Q2W).

HuAb1 may be administered to the subject in combination with a PD-(L)1 inhibitor. The PD-(L)1 inhibitor will be administered at the recommended dose and schedule. The PD-(L)1 inhibitor may be an anti-PD-1 antibody (e.g., cemiplimab, nivolumab, pembrolizumab, dostarlimab, toripalimab, tislelizumab, or retifanlimab) or an anti-PD-L1 antibody (e.g., atezolizumab, avelumab, or durvalumab).

HuAb1 may be administered to the subject in combination with a CTLA-4 inhibitor. The CTLA-4 inhibitor may be tremilimumab or ipilimumab.

HuAb1 may be administered to the subject in combination with a LAG-3 inhibitor. The LAG-3 inhibitor may be relatlimab.

HuAb1 may be administered to the subject in combination with an angiogenesis inhibitor. The angiogenesis inhibitor may be any angiogenesis inhibitor described herein. The angiogenesis inhibitor may be axitinib, tivozanib, vandetanib, nintedanib, sunitinib, or sorafenib. The angiogenesis inhibitor may be an anti-VEGF antibody (e.g., bevacizumab), anti-VEGF trap, or an anti-VEGF receptor antibody (e.g., ramucirumab).

HuAb1 may be administered to the subject in combination with a chemotherapeutic agent. The chemotherapeutic agent may be any chemotherapeutic agent described herein. The chemotherapeutic agent may be platinum chemotherapy (e.g., oxaliplatin, carboplatin, or cisplatin), trastuzumab, fluoropyrimidine, gemcitabine, irinotecan, 5-fluorouracil, or a taxane (e.g., docetaxel or paclitaxel).

HuAb1 may be administered to the subject in combination with immune-priming SBRT. The immune-priming SBRT is administered at a dose of 8 Gy on Day 1, Day 3, and Day 5 of each dosing cycle.

HuAb1 may be administered to the subject in combination with:
(a) a PD-(L)1 inhibitor, a CTLA-4 inhibitor, a LAG-3 inhibitor, an angiogenesis inhibitor, or a chemotherapeutic agent; and
(b) immune-priming SBRT.

HuAb1 may be administered to the subject in combination with:
(a) a CTLA-4 inhibitor, a LAG-3 inhibitor, an angiogenesis inhibitor, a chemotherapeutic agent, or immune-priming SBRT; and
(b) a PD-(L)1 inhibitor.

Following administration of the combination therapy to a patient, a practitioner of skill in the art can monitor the patient's response to the therapy by a variety of methods. For example, a physician can monitor the occurrence of dose-limiting toxicities (DLTs). A finding that the patient exhibits no DLTs indicates that the dose level of the combination therapy is safe and tolerable. Subsequent doses can be determined and administered as needed.

### Example 26. Treatment of NSCLC by Administration of an Anti-αvβ8 Antibody in Combination with a PD-(L)1 Inhibitor

According to the methods disclosed herein, a physician of skill in the art can treat a subject, such as a human patient, having a NSCLC. The patient will have had disease progression after at least one line of therapy or have no other standard therapy of proven clinical benefit currently available. To treat the subject, a physician of skill in the art can administer to the subject huAb1 in a combination therapy with a PD-(L)1 inhibitor.

HuAb1 may be administered to the subject intravenously (e.g., by intravenous infusion over 30±5 minutes) at a dose of about 1 mg/kg to about 30 mg/kg (e.g., about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, or about 25 mg/kg to about 30 mg/kg). HuAb1 may be administered on Day 1 of each 21-day dosing cycle (Q3W), or on Days 1 and 15 of each 28-day dosing cycle (Q2W).

HuAb1 may be administered to the subject in combination with a PD-(L)1 inhibitor. The PD-(L)1 inhibitor will be administered at the recommended dose and schedule. The PD-(L)1 inhibitor may be an anti-PD-1 antibody (e.g., cemiplimab, nivolumab, pembrolizumab, dostarlimab, toripalimab, tislelizumab, or retifanlimab) or an anti-PD-L1 antibody (e.g., atezolizumab, avelumab, or durvalumab).

Following administration of the combination therapy to a patient, a practitioner of skill in the art can monitor the patient's response to the therapy by a variety of methods. For example, a physician can monitor the overall success rate, which is defined as complete response (CR) or partial response (PR) of any duration, or stable disease (SD) that persists for at least 4 months. Subsequent doses can be determined and administered as needed.

### Example 27. Treatment of NSCLC, HNSCC, or Ovarian Cancer by Administration of an Anti-αvβ8 Antibody in Combination with a PD-(L)1 Inhibitor, or a PD-(L)1 Inhibitor and SBRT

According to the methods disclosed herein, a physician of skill in the art can treat a subject, such as a human patient, having a NSCLC, HNSCC, or ovarian cancer. The patient will have had disease progression after at least one line of therapy or have no other standard therapy of proven clinical benefit currently available. To treat the subject, a physician of skill in the art can administer to the subject huAb1 in a combination therapy according to one of the following:
NSCLC treated with huAb1, PD-(L)1 inhibitor and SBRT
HNSCC treated with huAb1 and PD-(L)1 inhibitor HNSCC treated with huAb1, PD-(L)1 inhibitor, and SBRT
Ovarian cancer treated with huAb1 and PD-(L)1 inhibitor.

HuAb1 may be administered to the subject intravenously (e.g., by intravenous infusion over 30±5 minutes) at a dose of about 1 mg/kg to about 30 mg/kg (e.g., about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, or about 25 mg/kg to about 30 mg/kg). HuAb1 may be administered on Day 1 of each 21-day dosing cycle (Q3W), or on Days 1 and 15 of each 28-day dosing cycle (Q2W).

The PD-(L)1 inhibitor is administered at the recommended dose and schedule. The PD-(L)1 inhibitor may be an anti-PD-1 antibody (e.g., cemiplimab, nivolumab, pembrolizumab, dostarlimab, toripalimab, tislelizumab, or retifanlimab) or an anti-PD-L1 antibody (e.g., atezolizumab, avelumab, or durvalumab).

The immune-priming SBRT is administered at a dose of 8 Gy on Day 1, Day 3, and Day 5 of each dosing cycle.

Following administration of the combination therapy to a patient, a practitioner of skill in the art can monitor the patient's response to the therapy by a variety of methods. For example, a physician can monitor the overall success rate, which is defined as complete response (CR) or partial response (PR) of any duration, or stable disease (SD) that persists for at least 4 months. Subsequent doses can be determined and administered as needed.

### OTHER EMBODIMENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims. Other embodiments are within the claims.

1. An antibody that binds to integrin αVβ8, the antibody comprising a light chain variable domain (VL) comprising an amino acid sequence of SEQ ID NO: 1 and a heavy chain variable domain (VH) comprising an amino acid sequence of SEQ ID NO: 2.

2. The antibody of claim 1, the antibody comprising a light chain amino acid sequence of SEQ ID NO: 3 and a heavy chain amino acid sequence of SEQ ID NO: 4.

3. A pharmaceutical composition comprising the antibody of claim 1 and a pharmaceutically acceptable carrier or excipient.

4. The pharmaceutical composition of claim 3, wherein the pharmaceutical composition is an aqueous formulation having a pH of between 5.0 and 7.5, and the antibody is present at a concentration of between 20 mg/mL and 40 mg/mL.

5. The pharmaceutical composition of claim 4, wherein the pharmaceutical composition comprises 10 to 30 mM phosphate buffer or histidine buffer.

6. The pharmaceutical composition of claim 4, wherein the pharmaceutical composition comprises 0.01% (w/w) to 0.1% (w/w) of a nonionic surfactant.

7-8. (canceled)

9. The pharmaceutical composition of claim 4, wherein the pharmaceutical composition comprises from 40 to 60 mM arginine or glutamate.

10. The pharmaceutical composition of claim 4, wherein the pharmaceutical composition comprises a nonionic tonicity agent.

11-17. (canceled)

18. A polynucleotide encoding the antibody of claim 1.

19. A vector comprising the polynucleotide of claim 18.

20-23. (canceled)

24. A host cell comprising the vector of claim 19.

25. A method of enhancing an immune response in a subject, the method comprising administering to the subject one or more dosing cycles of the antibody of claim 1.

26. The method of claim 25, wherein the subject has cancer and the antibody enhances the immune response against the cancer thereby treating the cancer.

27. The method of claim 26, wherein the cancer is a solid tumor, a metastatic cancer, a primary cancer, or an immune refractory cancer.

28-29. (canceled)

30. The method of claim 26, wherein the cancer is selected from lung cancer, optionally non-small cell lung cancer or small-cell lung cancer; head and neck cancer, optionally head and neck squamous cell carcinoma; renal cell carcinoma, optionally renal clear cell carcinoma or kidney renal papillary cell carcinoma; breast cancer, optionally triple-negative breast cancer; ovarian cancer; pancreatic cancer; brain cancer, optionally gliomas; colorectal cancer; urothelial cancer; bile duct cancer; endometrial cancer; melanoma; cervical cancer; gastric cancer; hepatocellular carcinoma; glioblastoma; or esophageal cancer.

31-37. (canceled)

38. The method of claim 26, further comprising administering to the subject an immune checkpoint inhibitor, a CTLA-4 inhibitor, a LAG-3 inhibitor, an anqioqenesis inhibitor, a chemotherapeutic agent, or immune-priming stereotactic body radiation therapy.

39. The method of claim 38, wherein the immune checkpoint inhibitor is a PD-(L)1 inhibitor.

40. The method of claim 39, wherein the PD-(L)1 inhibitor is an anti-PD-1 antibody.

41-58. (canceled)

59. The method of claim 25, wherein the method comprises administering to the subject the antibody at a dose of about 1 mg/kg to about 30 mg/kg.

60-65. (canceled)

66. The method of claim 25, wherein the length of each of the one or more dosing cycles is 21 days or 28 days.

67-75. (canceled)
